(19) United States
(12) Patent Application Publication
(10) Pub. No.: US 2024/0358226 A1
(43) Pub. Date: Oct. 31, 2024

(54) MEDICAL DEVICE WITH RENAL SHEATH WITH ADJUSTABLE VALVE

(71) Applicant: Boston Scientific Scimed, Inc., Maple Grove, MN (US)

(72) Inventors: Michael S.H. Chu, Brookline, MA (US); Jian Zhang, Lancaster, MA (US); Michael O'Brien, Hudson, MA (US); Gene Thomas Storbeck, Millis, MA (US)

(21) Appl. No.: 18/643,590

(22) Filed: Apr. 23, 2024

Related U.S. Application Data
(60) Provisional application No. 63/498,929, filed on Apr. 28, 2023.

(51) Int. Cl. A61B 1/00; A61B 1/015; A61B 1/018; A61B 1/307; A61B 17/22; A61B 18/00; A61B 18/26

(52) U.S. Cl. CPC A61B 1/00068; A61B 1/015; A61B 1/018; A61B 1/307; A61B 18/26; A61B 2017/22079; A61B 2018/00511

(57) ABSTRACT

In some aspects, a sheath device includes a sheath shaft defining a lumen. The sheath shaft is configured to be inserted into the body of a patient. The sheath device includes a valve assembly coupled to a proximal end portion of the sheath shaft. The valve assembly includes a connector and an elastic member. The elastic member is coupled to the connector. The connector includes an arm portion that extends from a sidewall of the connector. The elastic member is configured to operate as a valve. The elastic member defines a lumen.

### CROSS-REFERENCE TO RELATED APPLICATION

This application claims priority to U.S. Provisional Patent Application No. 63/498,929, filed on Apr. 28, 2023, entitled “MEDICAL DEVICE WITH RENAL SHEATH WITH ADJUSTABLE VALVE”, the disclosure of which is incorporated by reference herein in its entirety.

### TECHNICAL FIELD

This disclosure relates generally to medical devices, including a renal sheath with adjustable valve.

### BACKGROUND

Laser fibers may be inserted into a flexible ureteroscope to fragment and/or to dust a renal stone to a smaller size. However, removing stone fragments from the body of the patient may be difficult and/or time consuming. In some examples, a retrieval device may be used to remove the stone fragments from the body of the patient. However, in some examples, when a fragment is too large to fit through a working channel of the ureteroscope, the ureteroscope and the retrieval device may need to be withdrawn from the human body to remove the fragment. The ureteroscope and the retrieval device are then reinserted, to capture and remove the next fragment. This may become time-consuming and prolong the procedure.

### SUMMARY

In some aspects, a sheath device includes a sheath shaft defining a lumen. The sheath shaft is configured to be inserted into the body of a patient. The sheath device includes a valve assembly coupled to a proximal end portion of the sheath shaft. The valve assembly includes a connector and an elastic member. The elastic member is coupled to the connector. The connector includes an arm portion that extends from a sidewall of the connector. The elastic member is configured to operate as a valve. The elastic member defines a lumen.

In some aspects, the sheath shaft includes a distal end portion, and the distal end portion includes a laser-resistant material. In some examples, the sheath device includes an elongated member configured to be inserted to the lumen of the sheath shaft. The elongated member may include a dilator, a spacer, or a biasing member. In some examples, the biasing member includes a medical device. In some examples, the medical device includes a retrieval device. In some examples, the medical device includes a pressure and sensor device. The lumen of the elastic member may be configured to receive a shaft of an insertion device. In some examples, the arm portion is a first arm portion, and the connector includes a second arm portion that extends from the sidewall of the connector. The first arm portion may be curved, and the second arm portion may be straight. The valve assembly may include a cap member. The cap member is configured to move with respect to the connector to open or close the lumen of the elastic member. The cap member may include a cylindrical portion. The cylindrical portion may include a plunger defining a lumen. The plunger is configured to contact the elastic member. In some examples, the connector includes a snap ramp, and the cap member includes a snap hook configured to be engaged with the snap ramp. In some examples, the arm portion includes a governing hole. In some examples, the connector includes one or more cam plungers, and the cap member includes one or more cams.

In some aspects, a sheath device includes a sheath shaft defining a lumen. The sheath shaft is configured to be inserted into the body of a patient. The sheath device may include a biasing member having a first portion disposed within the sheath shaft. The sheath device may include a valve assembly coupled to a proximal end portion of the sheath shaft. The valve assembly includes a connector and an elastic member. The elastic member is coupled to the connector. The connector includes an arm portion that extends from a sidewall of the connector. A second portion of the biasing member may be disposed within the arm portion. The elastic member is configured to operate as a valve. The elastic member defining a lumen configured to receive a shaft of an insertion device.

In some aspects, the sheath shaft includes a distal end portion, and the distal end portion includes a laser-resistant material. In some examples, the distal end portion includes a transparent portion. In some examples, the sheath device includes a dilator defining a lumen, where the first portion of the biasing member is disposed within the lumen of the dilator. The arm portion may be a first arm portion and the connector may include a second arm portion. In some examples, the connector includes an indicator marking. In some examples, the indicator marking includes at least one of a line or an arrow that points to the line. In some examples, the indicator marking is distal to the arm portion, aligned with the arm portion, or proximal to the arm portion. The indicator marking may be molded or stamped onto the connector.

In some aspects, a method of inserting a sheath device into the body of a patient includes inserting a portion of a sheath device into the body of a patient, where the sheath device includes a sheath shaft, a connector having an arm portion, and an elastic member defining a lumen. The method may include inserting a shaft of an insertion device into a lumen of the sheath shaft via the lumen of the elastic member. The insertion device includes a laser fiber. The method may include activating the laser fiber and removing one or more stone fragments via the sheath shaft and the arm portion. In some examples, removing one or more stone fragments includes aspirating stone fragment(s) via the sheath shaft and the arm portion. The method may include moving a tip portion of the shaft of the insertion device to a location proximal to a tip portion of the sheath shaft and suctioning a stone into a distal end portion of the sheath shaft to fragment the stone using the laser fiber from a location within the distal end portion of the sheath shaft. The method may include extending a tip portion of the laser fiber to a location distal to a tip portion of the sheath shaft and activating the laser fiber to fragment a stone. The method may include inserting a biasing member into the sheath shaft via a second arm portion, where the biasing member is configured to bias the sheath shaft away from the shaft of the insertion device within the sheath shaft.

### BRIEF DESCRIPTION OF THE DRAWINGS

FIG. 1 illustrates a sheath device with a valve assembly according to an aspect.

FIG. 2A illustrates a side perspective of a sheath device with a dilator disposed within a lumen of a sheath device according to an aspect.

FIG. 2B illustrates a cross-sectional view of the sheath device taken along line A-A according to an aspect.

FIG. 2C illustrates an example of the dilator that may be used to insert a portion of the sheath device into the body of a patient according to an aspect.

FIG. 2D illustrates an example of a sheath shaft, a connector, an elastic member, and a cap member according to an aspect.

FIG. 2E illustrates an example of the connector according to an aspect.

FIG. 2F illustrates an example of the elastic member according to an aspect.

FIG. 2G illustrates an example of the cap member according to an aspect.

FIG. 2H illustrates an example of rotating the cap member, using a hand, to open or close a lumen of the elastic member according to an aspect.

FIG. 2I illustrates another example of rotating the cap member, using a hand, to open or close a lumen of the elastic member according to an aspect.

FIG. 2J illustrates another example of rotating the cap member, using a hand, to open or close a lumen of the elastic member according to an aspect.

FIG. 3A illustrates a side perspective of the sheath device with a spacer disposed within the lumen of the sheath device according to an aspect.

FIG. 3B illustrates a cross-sectional view of the sheath device taken along line B-B according to an aspect.

FIG. 3C illustrates an example of the spacer that is used to insert a portion of the sheath device into the body of a patient according to an aspect.

FIG. 3D illustrates an example of a sheath shaft, a compression cap, a connector, an elastic member, and a cap member according to an aspect.

FIG. 3E illustrates an example of the compression cap according to an aspect.

FIG. 3F illustrates an example of the connector according to an aspect.

FIG. 3G illustrates an example of the elastic member according to an aspect.

FIG. 3H illustrates an example of the cap member according to an aspect.

FIG. 4A illustrates a side perspective of the sheath device with the spacer disposed within the lumen of the sheath device according to an aspect.

FIG. 4B illustrates a cross-sectional view of the sheath device taken along a line C-C according to an aspect.

FIG. 4C illustrates an example of the spacer that is used to insert a portion of the sheath device into the body of a patient according to an aspect.

FIG. 4D illustrates an example of the sheath shaft, the connector, the elastic member, and the cap member according to an aspect.

FIG. 4E illustrates a plurality of cams on the cap member according to an aspect.

FIG. 4F illustrates an example of the connector according to an aspect.

FIG. 4G illustrates an example of the elastic member according to an aspect.

FIG. 4H illustrates an example of the cap member according to an aspect.

FIG. 5A illustrates a side perspective of the sheath device with the dilator disposed within the lumen of the sheath device according to an aspect.

FIG. 5B illustrates a cross-sectional view of the sheath device taken along a line D-D according to an aspect.

FIG. 5C illustrates an example of the dilator that is used to insert a portion of the sheath device into the body of a patient according to an aspect.

FIG. 5D illustrates an example of the sheath shaft, the connector, the elastic member, and the cap member according to an aspect.

FIG. 5E illustrates an example of the connector according to an aspect.

FIG. 5F illustrates an example of the elastic member configured to operate as a valve according to an aspect.

FIG. 5G illustrates an example of the cap member according to an aspect.

FIG. 6A illustrates a side perspective of the sheath device with the dilator disposed within the lumen of the sheath device according to an aspect.

FIG. 6B illustrates a cross-sectional view of the sheath device taken along a line E-E according to an aspect.

FIG. 6C illustrates an example of the dilator that is used to insert a portion of the sheath device into the body of a patient according to an aspect.

FIG. 6D illustrates an example of the sheath shaft, the connector, the elastic member, and the cap member according to an aspect.

FIG. 6E illustrates a perspective of the cap member having a cavity according to an aspect.

FIG. 6F illustrates an example of the connector according to an aspect.

FIG. 6G illustrates an example of the elastic member according to an aspect.

FIG. 6H illustrates an example of the cap member according to an aspect.

FIG. 7A illustrates a side perspective of the sheath device with the dilator disposed within the lumen of the sheath device according to an aspect.

FIG. 7B illustrates a cross-sectional view of the sheath device taken along a line F-F according to an aspect.

FIG. 7C illustrates an example of the dilator that is used to insert a portion of the sheath device into the body of a patient according to an aspect.

FIG. 7D illustrates an example of the sheath shaft, the connector, the elastic member, the retainer, the spring member, and the cap member according to an aspect.

FIG. 7E illustrates an example of the connector according to an aspect.

FIG. 7F illustrates an example of the elastic member according to an aspect.

FIG. 7G illustrates an example of the retainer according to an aspect.

FIG. 7H illustrates an example of the spring member according to an aspect.

FIG. 7I illustrates an example of the cap member according to an aspect.

FIG. 8A illustrates an unassembled perspective of the connector, the elastic member, and the cap member according to an aspect.

FIG. 8B illustrates an assembled perspective of the connector, the elastic member, and the cap member according to an aspect.

FIG. 9A illustrates an unassembled perspective of the compression cap, connector, the elastic member, and the cap member according to an aspect.

FIG. 9B illustrates an assembled perspective of the compression cap, the connector, the elastic member, and the cap member according to an aspect.

FIG. 10A illustrates a perspective of the sheath device with a sheath shaft disposed in the kidney according to an aspect.

FIG. 10B illustrates a side perspective of the sheath device with the sheath shaft disposed in the kidney according to an aspect.

FIG. 10C illustrates an engaged view of the tip of the sheath shaft according to an aspect.

FIG. 10D illustrates a cross-sectional view of the sheath shaft according to an aspect.

FIG. 11A illustrates a side perspective of the sheath device with the sheath shaft disposed in the kidney according to an aspect.

FIG. 11B illustrates an enlarged view of the tip of the sheath shaft according to an aspect.

FIG. 12A illustrates a side perspective of the sheath device with the sheath shaft disposed in the kidney according to an aspect.

FIG. 12B illustrates an enlarged view of the tip of the sheath shaft according to an aspect.

FIG. 13A illustrates a sheath device with a dual sheath shaft and a third sidearm according to an aspect.

FIG. 13B illustrates a sheath device with a dual sheath shaft and a secondary connector according to an aspect.

FIG. 13C illustrates two sheath devices coupled together according to an aspect.

FIG. 13D illustrates two sheath devices coupled together according to another aspect.

FIG. 13E illustrates a cross-sectional profile of a dual sheath shaft according to an aspect.

FIG. 14 illustrates an example of a system having multiple sheath devices connected in series according to an aspect.

FIG. 15 illustrates a sheath device according to another aspect.

FIG. 16A illustrates the dilator according to an aspect.

FIG. 16B illustrates the biasing member according to an aspect.

FIG. 16C illustrates a side view of the sheath device according to an aspect.

FIG. 16D illustrates an example of the sheath device with the dilator according to an aspect.

FIG. 16E illustrates an example of the sheath device with the biasing member according to an aspect.

FIG. 16F illustrates the sheath device according to another aspect.

FIG. 16G illustrates a cross-section of the sheath shaft that includes the biasing member, a scope shaft, and a gap according to an aspect.

FIG. 16H depicts a sheath tip in the body of the patient according to an aspect.

FIG. 16I illustrates the distal tip of the scope shaft extending from the sheath tip according to an aspect.

FIG. 16J illustrates a perspective of the scope tip and the sheath tip in the kidney of the patient according to an aspect.

FIG. 16K illustrates a perspective of the scope tip and the sheath tip in the kidney of the patient according to an aspect.

FIG. 16L illustrates a perspective of the scope tip, a laser fiber, and the sheath tip in the kidney of the patient according to an aspect.

FIG. 16M illustrates a perspective of collecting stone fragments in the sheath shaft according to an aspect.

FIG. 17 illustrates a perspective of the sheath device according to an aspect.

FIG. 18A illustrates an example of the medical device with a sheath shaft disposed in a kidney and a scope shaft disposed within the lumen of the sheath shaft according to an aspect.

FIG. 18B illustrates an exploded view of the sheath assembly according to an aspect.

FIG. 18C illustrates a packaged configuration of the sheath assembly according to an aspect.

FIG. 18D illustrates a packaged configuration of the sheath assembly according to another aspect.

FIG. 18E illustrates a perspective of the scope shaft being inserted into the sheath shaft when the sheath shaft is disposed in the body of the patient according to an aspect.

FIG. 18F illustrates an enlarged portion of a distal end portion of the sheath shaft according to an aspect.

FIG. 18G illustrates a cross-sectional view of the sheath shaft taken along line G-G according to an aspect.

FIG. 18H illustrates a perspective of the sheath device in which a tip portion of a scope shaft extends away from the distal end portion of the sheath shaft according to an aspect.

FIG. 18I illustrates a perspective of the sheath device in which a tip portion of the scope shaft is proximate to the distal end portion of the sheath shaft according to an aspect.

FIG. 18J illustrates an enlarged view of the distal end portion of the sheath shaft according to an aspect.

FIG. 18K illustrates a perspective of the sheath device when the tip portion of the scope shaft is withdrawn to a withdrawal stop marker according to an aspect.

FIG. 18L illustrates an enlarged view of a portion of the sheath device according to an aspect.

FIG. 18M illustrates a cross-sectional view of the sheath shaft taken along line M-M according to an aspect.

FIG. 19A illustrates a perspective of a sheath device in which stones and/or debris may be lasered inside of the inner diameter of the sheath or at the edge of the sheath according to an aspect.

FIG. 19B illustrates a perspective of a sheath device that is used to laser a stone located distal to the sheath according to an aspect.

FIG. 19C illustrates a perspective of a sheath device in which the scope shaft is withdrawn to allow a larger fragment to be aspirated according to an aspect.

FIG. 20 illustrates a flowchart of inserting a sheath device into the body of a patient according to an aspect.

### DETAILED DESCRIPTION

This disclosure relates to a sheath device configured to be used with an insertion device (e.g., a ureteroscope) and/or one or more elongated devices (e.g., biasing member, dilator, guidewire, laser fiber, etc.), where the insertion device and/or the elongated device(s) may be inserted into the sheath device. In some examples, the elongated devices may also include pressure sensor(s), temperature sensor(s), optical/digital imager(s), baskets, retrieval device(s), snare(s), tube(s), and catheter(s). The insertion device and/or the elongated devices may be moved inside the sheath device to treat and aspirate renal stones, stone fragments, stone dust and other debris such as tissue, blood, and fluids or foreign debris from the human body such as the kidney and the urinary tract.

The sheath device member may define a lumen, where at least a portion of the insertion device and/or the elongated member(s) may be disposed within the lumen and may move with respect to the sheath device. The sheath device includes a sheath shaft and a valve assembly. The sheath shaft includes a distal portion that is flexible to traverse or bend around a ureteropelvic junction and may be advanced into the kidney or urinary tract. In some examples, the sheath shaft includes a laser resistant sheath. In some examples, a distal portion of the inner diameter of the sheath shaft includes an expanded polytetrafluoroethylene (EPTFE) material.

The valve assembly may be connected to a proximal end portion of the sheath shaft. The valve assembly may include a connector (e.g., a connector with three or more outlets). The valve assembly may include an elastic member configured to operate as a valve. In some examples, the valve assembly includes a cap member. In some examples, movement (e.g., rotation) of the cap member causes a lumen of the elastic member to open or close, thereby opening or sealing the valve. In some examples, the valve assembly does not include a cap member. The lumen of the elastic member is configured to receive the insertion device and/or the elongated device(s). In some examples, the elastic member is configured to operate in a range of positions between an open position (e.g., fully open) and a closed position (e.g., fully sealed). In the open position, elongated member(s) and/or fluid/debris may pass through the lumen of the elastic member. In the closed position, the elongated member(s) and/or fluid/debris material is restricted or prevented from moving through the elastic member. The valve assembly may be operated with a single hand or both hands.

In some examples, the sheath shaft is delivered into the body via an insertion device (e.g., a ureteroscope) and/or a dilator or other elongated member. A distal end portion of the insertion device may be advanced or extended beyond the sheath tip. In some examples, the distal end portion of the insertion device may be retracted into the sheath shaft. In some examples, a tip of the laser fiber may extend from the insertion device and the tip of the laser fiber may be included in the lumen of the sheath shaft. In some examples, stone fragments may be suctioned into the tip of the sheath shaft, where activation of the laser fiber causes the stone fragments to break apart and be suctioned through a working channel of the sheath shaft. In some examples, the distal end of the sheath shaft is steered by the distal end of the insertion device to be positioned in proximity or just proximal to a renal stone in the kidney for treatment. Aspiration of renal stones and debris may improve and quicken the removal/retrieval process from the kidney as multiple fragments/dust may be aspirated at one time.

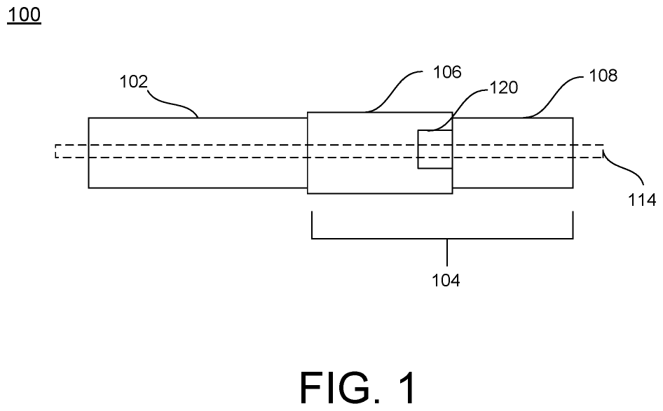

FIG. 1 illustrates an example of a sheath device 100 configured to facilitate the removal of stones, stone fragments, or other debris from the human body (e.g., the kidney, urinary tract, etc.). The sheath device 100 may be an example of any of the sheath devices discussed herein and may include any of the details discussed with reference to the other figures. In some examples, the sheath device 100 may be configured to be used with an insertion device 114 and/or one or more elongated members (e.g., a biasing member, a guidewire, a dilator, a tube, etc.). In some examples, the insertion device 114 is a ureteroscope. In some examples, the insertion device 114 includes a laser fiber configured to extend from a distal tip of the insertion device 114.

The sheath device 100 includes a sheath shaft 102 and a valve assembly 104. The valve assembly 104 includes a connector 106, an elastic member 120 (e.g., a valve), and a cap member 108. In some examples, the valve assembly 104 does not include a cap member 108. For example, the valve assembly 104 may include the connector 106 and the elastic member 120. The sheath shaft 102 may be coupled to the valve assembly 104. In some examples, the sheath shaft 102 is fixedly coupled (e.g., permanently) to the valve assembly 104. In some examples, the sheath shaft 102 is removably coupled to the valve assembly 104. In some examples, the insertion device 114 is inserted into a lumen of the sheath shaft 102, and the insertion device 114 and the sheath shaft 102 are inserted into the body of the patient.

The sheath shaft 102 includes a distal end portion and a proximal end portion. The sheath shaft 102 defines a lumen that extends between (and through) the distal end portion and the proximal end portion. The proximal end portion is configured to connect to a distal end portion of the connector 106 of the valve assembly 104.

The sheath shaft 102 may include a tubular member. In some examples, the sheath shaft 102 includes a circular profile. The sheath shaft 102 may include an inner surface defining an inner diameter (and a lumen) and an outer surface defining an outer diameter. The sheath shaft 102 may include one or more polymer materials. In some examples, the sheath shaft 102 includes one or multiple layers of polymer materials. In some examples, the sheath shaft 102 includes a flexible portion and/or a rigid portion. In some examples, the inner surface (e.g., inner layer) of the sheath shaft includes a lubricious material such as Polytetrafluoroethylene (PTFE) or other types of lubricious materials. In some examples, the sheath shaft 102 includes a reinforcement member. In some examples, the reinforcement member includes a braid. In some examples, the reinforcement member includes a coil. In some examples, the sheath shaft 102 includes one or more laser-resistant materials.

In some examples, the valve assembly 104 is a single integral body configured to be coupled to the proximal end portion of the sheath shaft 102. In some examples, the valve assembly 104 includes multiple components that are coupled together. In some examples, the valve assembly 104 includes a connector 106, a cap member 108, and an elastic member 120 configured to operate as a valve that opens and closes. In some examples, the cap member 108 is a rotatable member on the connector 106. In some examples, the connector 106, the cap member 108, and the elastic member 120 are separate components that are coupled together. In some examples, the valve assembly 104 does not include the cap member 108. In some examples, the connector 106 is coupled to the proximal end portion of the sheath shaft 102. The elastic member 120 may be disposed within a cavity of the connector 106. In some examples, the cap member 108 is rotatably coupled to the connector 106 (e.g., screwed on the connector 106). In some examples, the cap member 108 is coupled to the connector 106 based on a snap fit. By moving (e.g., moving in a certain direction, rotating, etc.) the cap member 108, the cap member 108 may compress or expand the elastic member 120, thereby closing or opening the valve.

The connector 106 may include one or more polymer materials. In some examples, the connector 106 includes a transparent material such as polycarbonate. In some examples, the connector 106 is a single integral component. In some examples, the connector 106 includes multiple components that are coupled together. In some examples, the connector 106 includes a Y-shaped fitting and a valve housing.

The connector 106 may include a connector portion and an arm portion. The arm portion may branch (e.g., extend) from the connector portion. In some examples, the connector 106 includes a Y connector with a valve housing. In some examples, the connector includes more than one arm portion such as two, three, four arm portions. The connector portion includes a distal end portion and a proximal end portion. The distal end portion of the connector portion is configured to connect to the proximal end portion of the sheath shaft 102, and the proximal end portion of the connector portion is configured to connect to the cap member 108. In some examples, the connector 106 includes threads, e.g., external threads, defined on the outer surface of the connector 106.

In some examples, another elongated member (e.g., a smaller diameter) is configured to pass through the lumen of the arm portion. In some examples, the arm portion includes a curved portion. In some examples, the arm portion is configured to be coupled to a device that may deliver or generate negative pressure (e.g., a suctioning device). In some examples, the arm portion includes a hole. Covering or partially covering the hole with a finger may increase the negative pressure while uncovering the hole may decrease the negative pressure through the lumen of the sheath shaft 102. In some examples, the arm portion is used for the injection of a fluid between the outer diameter of an elongated device and an inner diameter of the sheath shaft 102. In some examples, the arm portion does not include a hole. In some examples, the sheath assembly fluid inlet and outlet may be controlled by a fluid management system (FMS) and a pressure and temperature sensor. In some examples, an elongated device (e.g., an insertion device) may have at least one lumen that may be used as either an inlet lumen or an outlet to be the opposite flow direction of the sheath shaft 102 to have a continuous aspiration flow. For example, if the sheath device 100 has a negative pressure outlet flow, the insertion device (e.g., working channel of a ureteroscope) may have a positive inlet flow.

The elastic member 120 is configured to operate as a valve. The elastic member 120 defines a lumen (e.g., through lumen) that is configured to be compressed to seal the valve (e.g., prevent the transfer of fluid/debris through the lumen) or lock onto the insertion device 114 and one or more elongated devices. In some examples, the lumen of the elastic member 120 may have a round (e.g., circular) cross-sectional shape. In some examples, the lumen of the elastic member 120 may have other cross-sectional shapes such as oval, hexagon, polygon, square, or a star.

In some examples, the connector 106 includes one or more wings. In some examples, the connector 106 includes one or more fins. In some examples, the connector 106 includes one or more ring members.

The cap member 108 is configured to be coupled (e.g., snap-fit, rotatably coupled, etc.) to the connector 106. In some examples, the cap member 108 includes a polymer-based material. In some examples, the cap member 108 includes a polycarbonate material. The cap member 108 may include a tubular member. In some examples, the cap member 108 includes a cylindrical portion. In some examples, the cap member 108 includes a plunger that is disposed within the cavity defined by the inner surface of the cap member 108. In some examples, the cap member 108 includes one or more cams configured to engage with one or more cam plungers on the connector 106. In some examples, the plunger includes an inner tubular member, and the inner tubular member defines a lumen configured to receive a portion of the insertion device 114. In some examples, the cap member 108 may include one or more fins. In some examples, the cap member 108 includes one or more ring members. In some examples, the cap member 108 includes a funnel. In some examples, the funnel includes a holder clip. In some examples, the funnel defines a funnel slot.

In some examples, the connector 106 and the cap member 108 are configured to be operated using a single hand while the other hand is used to operate the control handle of the scope or another elongated device. Both hands, if available, may be used to operate the connector 106 and the cap member 108 to open or close the elastic member 120. In some examples, a portion of the first hand is used to secure the connector 106 (e.g., prevent the connector 106 from rotating) while the thumb is used to rotate the cap member 108. In some examples, the index finger of the first hand is also used to aid in rotating the cap member 108. In some examples, securing (e.g., locking) the sheath device 100 to a steerable device shaft (e.g., insertion device 114) allows for both devices to move in unison by moving either the insertion device 114 or the sheath device 100. The lock may also maintain the extended or retracted position of the sheath device 100 relative to the steerable device.

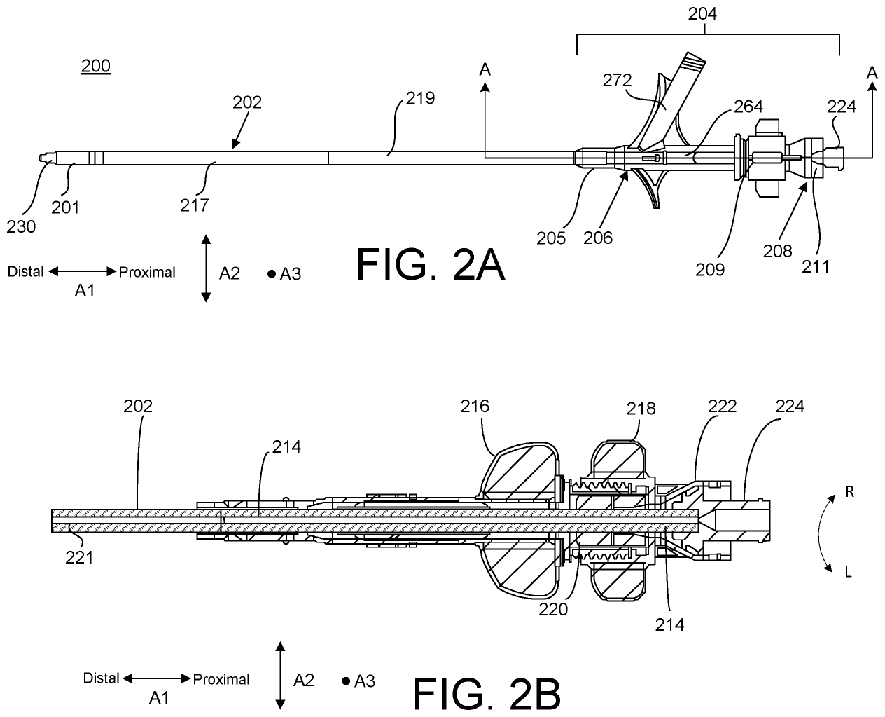

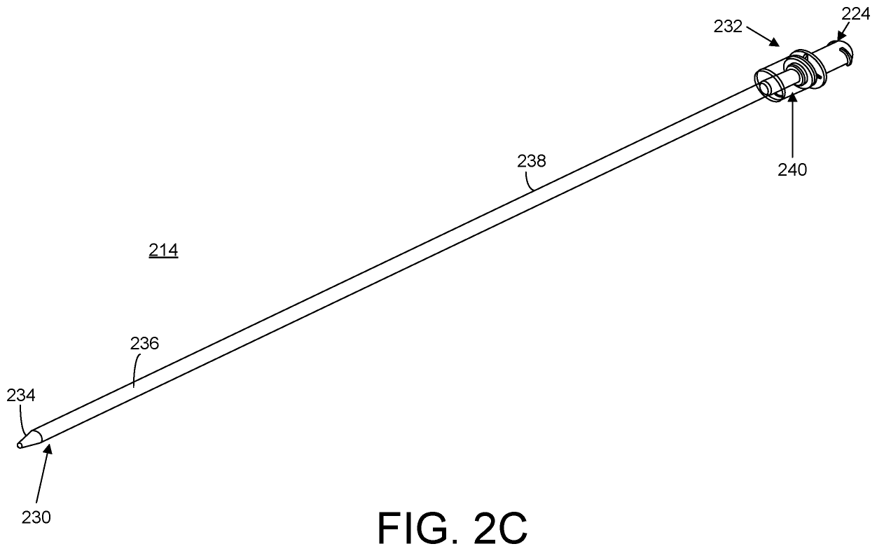

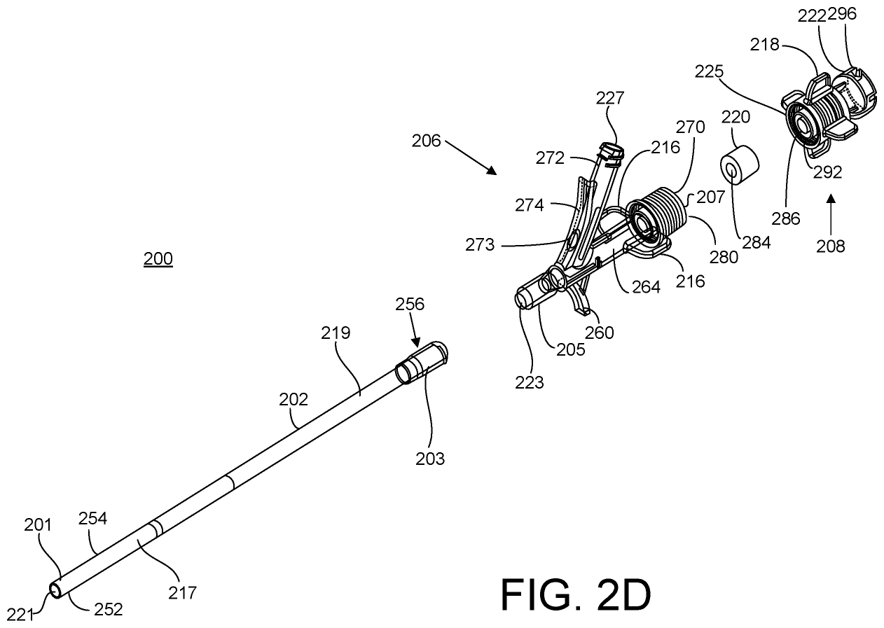

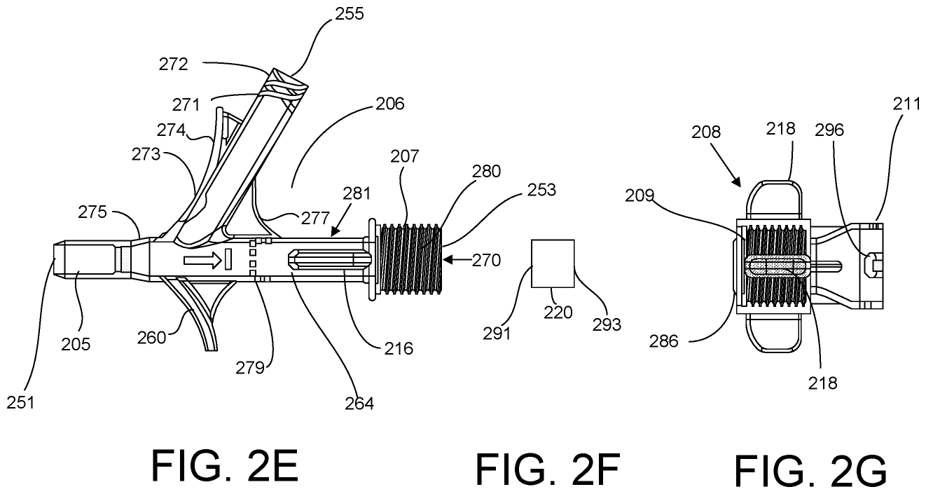

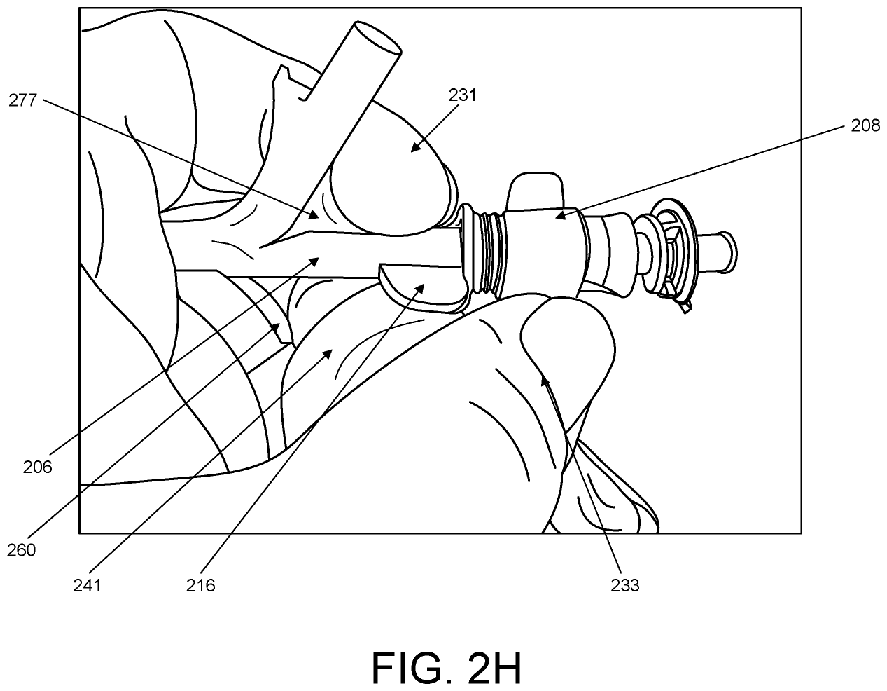

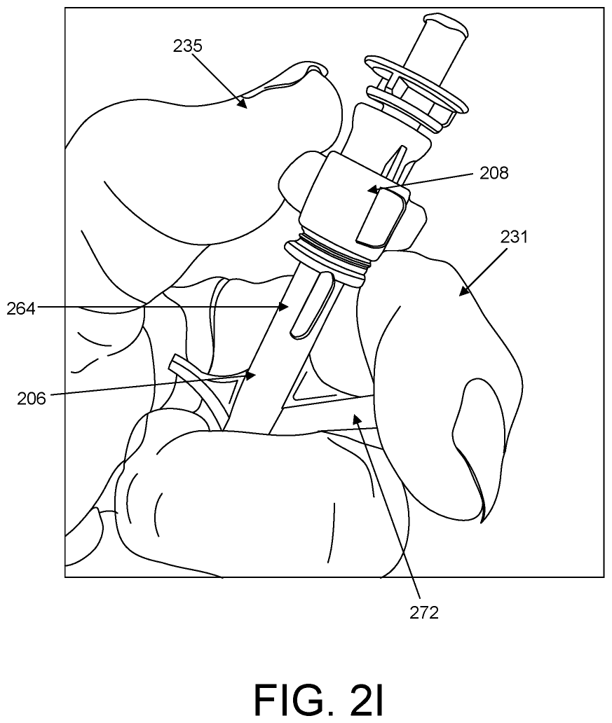

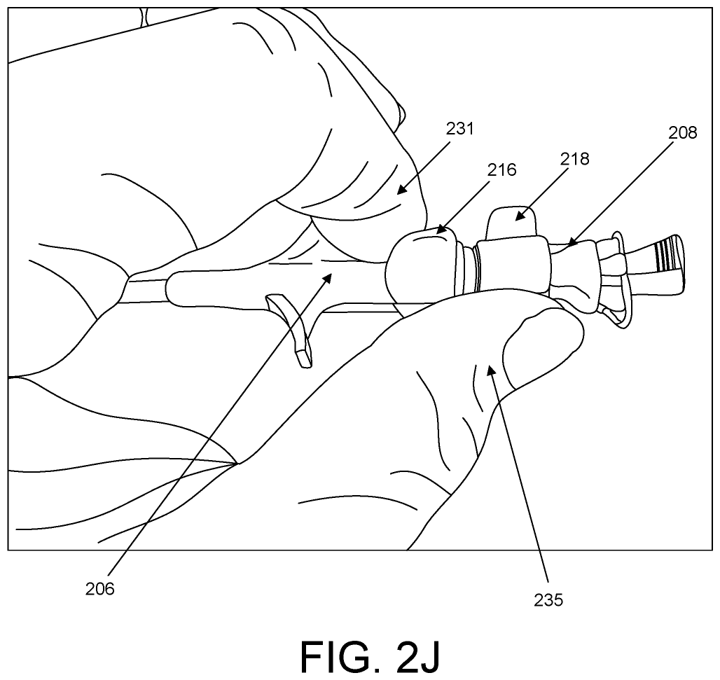

FIGS. 2A through 2J illustrate an example of a sheath device 200 configured to facilitate the removal of stones, stone fragments, or other debris from the human body (e.g., the kidney, urinary tract, etc.). The sheath device 200 may be an example of any of the sheath devices discussed herein and may include any of the details discussed with reference to the other figures. In some examples, the sheath device 200 may be configured to be used with a dilator 214 to place a portion of the sheath device 200 into the human body (e.g., the kidney, urinary tract).

FIG. 2A illustrates a side perspective of the sheath device 200 with the dilator 214 disposed within the lumen of the sheath device 200. FIG. 2B illustrates a cross-sectional view of the sheath device 200 taken along line A-A. FIG. 2C illustrates an example of the dilator 214 that is used to insert a portion of the sheath device 200 into the body of a patient. In some examples, the dilator 214 is an example of an elongated device. The sheath device 200 includes a sheath shaft 202 and a valve assembly 204. The valve assembly 204 includes a connector 206, an elastic member 220 (e.g., a valve), and a cap member 208. FIG. 2D illustrates an example of the sheath shaft 202, the connector 206, the elastic member 220, and the cap member 208. FIG. 2E illustrates an example of the connector 206. FIG. 2F illustrates an example of the elastic member 220 configured to operate as a valve. FIG. 2G illustrates an example of the cap member 208. FIG. 2H illustrates an example of rotating the cap member 208, using a hand, to open or close a lumen 284 of the elastic member 220, thereby opening or closing the valve. FIG. 2I illustrates another example of rotating the cap member 208, using a hand, to open or close a lumen 284 of the elastic member 220. FIG. 2J illustrates another example of rotating the cap member 208, using a hand, to open or close a lumen 284 of the elastic member 220.

The sheath device 200 includes a sheath shaft 202 and a valve assembly 204. The sheath shaft 202 may be coupled to the valve assembly 204. In some examples, the sheath shaft 202 is fixedly coupled (e.g., permanently) to the valve assembly 204. In some examples, the sheath shaft 202 is removably coupled to the valve assembly 204. In some examples, the dilator 214 is inserted into a lumen 221 of the sheath shaft 202, and the dilator 214 and the sheath shaft 202 are inserted into the body of the patient. When in the appropriate location in the body of the patient, the dilator 214 is removed, and the valve assembly 204 is coupled to the sheath shaft 202. In some examples, the dilator 214 is inserted through the lumens (e.g., lumen 221, lumen 223, lumen 284, lumen 225) of the sheath device 200. The valve assembly 204 may be actuated to close the lumen 284 of the elastic member 220, thereby securing (e.g., gripping) the dilator 214. Then, the dilator 214 and the sheath shaft 202 are inserted into the body of the patient with the valve assembly 204 attached to the sheath shaft 202. In some examples, before, during and/or after insertion of the sheath shaft 202 into the body, the lumens of the sheath device 200 may include other elongated devices such as a scope (e.g., a ureteroscope), a guidewire, and/or one or more laser fibers, etc.

The sheath shaft 202 includes a distal end portion 201 and a proximal end portion 203. The sheath shaft 202 defines a lumen 221 that extends between (and through) the distal end portion 201 and the proximal end portion 203. The proximal end portion 203 is configured to connect to a distal end portion 205 of the connector 206 of the valve assembly 204. In some examples, the proximal end portion 203 includes a hub connector 256. In some examples, the hub connector 256 includes a male hub connector. In some examples, the hub connector 256 includes a female hub connector. In some examples, the proximal end portion 203 does not include a hub connector 256. In some examples, the hub connector 256 is connected to the outer surface of the sheath shaft 202 at the proximal end portion 203. In some examples, the hub connector 256 is configured to connect to a corresponding hub connector on the distal end portion 205 of the connector 206.

The sheath shaft 202 may include a tubular member. In some examples, the sheath shaft 202 includes a circular profile. The sheath shaft 202 may include an inner surface defining an inner diameter (and the lumen 221) and an outer surface defining an outer diameter. Referring to FIGS. 2A and 2B, the sheath shaft 202 has a length that extends in a direction A1. The direction A1 may be referred to as a distal direction or a proximal direction. A direction A2 is perpendicular to the direction A1. A direction A3 (represented by a black dot) extends into (and out of) the page and is orthogonal to directions A1 and A2.

The sheath shaft 202 may include a distal portion 217 and a proximal portion 219. In some examples, the sheath shaft 202 is divided into a distal portion 217 and a proximal portion 219. The distal portion 217 is a portion of the sheath shaft 202 that includes the distal end portion 201. The proximal portion 219 is a portion of the sheath shaft 202 that includes the proximal end portion 203. In some examples, the length of the distal portion 217 is the same as the length of the proximal portion 219. In some examples, the length of the distal portion 217 is greater than the length of the proximal portion 219. In some examples, the length of the proximal portion 219 is greater than the length of the distal portion 217.

The sheath shaft 202 may include one or more polymer materials. In some examples, the sheath shaft 202 includes one or multiple layers of polymer materials. In some examples, the distal portion 217 and the proximal portion 219 include different polymer materials and/or different properties of polymer materials. In some examples, the distal portion 217 includes a flexible portion 254 that has a rigidity (e.g., stiffness) that is less than the rigidity of the proximal portion 219. In some examples, the flexible portion 254 is more flexible than the proximal portion 219. In some examples, the distal portion 217 of the sheath shaft 202 is flexible with minimum recoil (e.g., tendency to revert to a straight condition). In some examples, the proximal portion 219 of the sheath shaft 202 is semi-rigid. In some examples, the inner surface (e.g., inner layer) of the sheath shaft 202 that defines the lumen 221 includes a lubricious material such as Polytetrafluoroethylene (PTFE) or other types of lubricious materials.

In some examples, the sheath shaft 202 includes a secondary layer of polymer that is reinforced with a braid and/or a coil at the distal end portion 201 and/or at the proximal end portion 203. In some examples, the coil and braid are constructed from polymer(s) materials such as Pebax®. In some examples, the coil and braid are constructed from one or more metal materials such as stainless steel. In some examples, the coil and braid are constructed from a nitinol material in the form of flat or round wire. In some examples, the sheath shaft 202 includes another layer of polymer, e.g., an outer sheath layer. In some examples, the sheath outer layer may be constructed from another polymer material (e.g., Tecoflex®, Tecothane®, etc.) at the distal end portion 201. In some examples, the sheath outer layer may be constructed from a semi-rigid Pebax® material at the proximal end portion 203. In some examples, the outer surface of the sheath shaft 202 may include a radiopaque material. In some examples, the sheath shaft 202 includes a radiopaque marker 252 at the distal end portion 201. In some examples, the radiopaque marker 252 may be located on the inner surface of the sheath shaft 202 to indicate the distal tip of the sheath shaft 202 when viewed with a fluoroscope.

In some examples, the sheath shaft 202 includes one or more laser resistant materials (e.g., the sheath shaft 202 is a laser-resistant sheath). In some examples, the laser is activated when inside the lumen of the sheath shaft 202 to fragment or dust the stone or stone fragments. In some examples, a laser resistant material includes expanded polytetrafluoroethylene (EPTFE). EPTFE may be microporous and is different (e.g., significantly different) to conventional PTFE tubing. In some examples, EPTFE is air-permeable, and relatively soft and flexible. In some examples, a laser resistant material may include other polymer materials such as polytetrafluoroethylene (PTFE), tetrafluoroethylene (TFE), fluorinated ethylenepropylene (FEP), perfluoroalkoy (PFA), ethylene tetra-fluoroethylene (ETFE), and polyvinylidene fluoride (PVDF).

In some examples, the inner layer or inner diameter of the sheath shaft 202 may be an EPTFE material or other laser resistant material. In some examples, a laser-resistant tube or catheter may be inserted into the lumen 221 of the sheath shaft 202 and the tube or catheter may extend between the proximal end portion 203 and the distal end portion 201 (e.g., along the entire length of the sheath shaft 202). In some examples, a portion of the sheath shaft 202 is laser resistant (e.g., the distal portion 217 of the sheath shaft 202 is laser resistant), where a laser resistant tube may be attached or bonded to the inside of the distal portion 217 of the sheath shaft 202.

The inner diameter of the sheath shaft 202 may be greater than an outer diameter of the flexible ureteroscope or steerable device to accommodate additional elongated devices to be passed alongside the scope and/or to provide an aspiration (inlet or outlet) channel between the inner diameter of the sheath shaft 202 and the outer diameter of the steerable device.

The sheath shaft 202 (or a portion thereof) may be placed in the human body using a dilator 214. The dilator 214 may be an elongated member. In some examples, the dilator 214 is a tubular member having a length that extends in the direction A1. As shown in FIG. 2C, the dilator 214 includes a distal end portion 230 and a proximal end portion 232. In some examples, the distal end portion 230 includes a tapered portion. In some examples, the dilator 214 includes a hub connector 224 at the proximal end portion 232 of the dilator 214. In some examples, the hub connector 224 includes a cylindrical portion that has a size that is larger than the outer diameter of the sheath shaft 202. In some examples, the hub connector 224 is configured to be disposed in a funnel 222 of the cap member 208 when the dilator 214 is inserted into the sheath shaft 202. In some examples, the hub connector 224 is configured to contact (e.g., engaged with) a holder clip 296 on the funnel 222 of the cap member 208. In some examples, the dilator 214 does not include a hub connector 224.

In some examples, the dilator 214 includes a hub connector 240 (e.g., instead of the hub connector 224). In some examples, the hub connector 240 includes a male connector. In some examples, the hub connector 240 includes a female connector. In some examples, the hub connector 240 is configured to engage with the hub connector 256 on the proximal end portion 203 of the sheath shaft 202. In some examples, the hub connector 240 is a male connector, and the hub connector 256 is a female connector, which, when connected, locks the sheath shaft 202 and the dilator 214 together. In some examples, the sheath shaft 202 and the dilator 214 are inserted over a guidewire. Once in the appropriate location in the human body, the hub connector 240 and the hub connector 256 may be unscrewed (unlocked) to remove the dilator 214, thereby leaving the sheath shaft 202 behind. The valve assembly 204 (e.g., the connector 206, the elastic member 220, and the cap member 208) may then be attached to the sheath shaft 202 for use.

The dilator 214 may have an inner surface that defines an inner diameter and an outer surface that defines an outer diameter. In some examples, the dilator 214 defines a lumen 234. In some examples, the outer diameter of the dilator 214 is equal to or less than the inner diameter of the sheath shaft 202. The dilator 214 may have a flexible portion 236 such that the flexible portion 236 may bend about the ureteropelvic junction to guide the sheath shaft 202 directly in the kidney. In some examples, the flexible portion 236 is defined on a distal portion of the dilator 214. In some examples, the dilator 214 is inserted into the sheath device 200 and is locked together via the elastic member 220 and inserted over a guidewire to be placed. In some examples, the dilator 214 includes a rigid portion 238. In some examples, the rigid portion 238 is a stiffness greater than the flexible portion 236. In some examples, the rigid portion 238 is defined on a proximal portion of the dilator 214. Once the sheath shaft 202 is in the appropriate location, the elastic member 220 is unlocked, and the dilator 214 removed, leaving the sheath device 200 behind for use.

In some examples, the valve assembly 204 is a single integral body configured to be coupled to the proximal end portion 203 of the sheath shaft 202. In some examples, the valve assembly 204 includes multiple components that are coupled together. In some examples, the valve assembly 204 includes a connector 206, a cap member 208, and an elastic member 220 configured to operate as a valve that opens and closes. In some examples, the connector 206, the cap member 208, and the elastic member 220 are separate components that are coupled together. In some examples, the connector 206 is coupled to the proximal end portion 203 of the sheath shaft 202. The elastic member 220 may be disposed within a cavity 270 of the connector 206. In some examples, the cap member 208 is rotatably coupled to the connector 206 (e.g., screwed on the connector 206). By moving (e.g., rotating) the cap member 208, the cap member 208 may compress or expand the elastic member 220, thereby closing or opening the valve.

The connector 206 may include one or more polymer materials. In some examples, the connector 206 includes a transparent material such as polycarbonate. In some examples, the connector 206 is a single integral component. In some examples, the connector 206 includes multiple components that are coupled together. In some examples, as shown in FIG. 2E, the connector 206 includes a fitting 275 (e.g., a Y-shaped fitting) and a valve housing 281. The dotted line in FIG. 2E represents a connection 279 between the fitting 275 and the valve housing 281. In some examples, the fitting 275 and the valve housing 281 are coupled together using an adhesive material (e.g., bonding material such as glue) or other types of fasteners (e.g., screw heads). In some examples, the connector 206 is a Y-connector (e.g., a connector with three openings).

The connector 206 may include a connector portion 264 and an arm portion 272. The arm portion 272 may branch (e.g., extend) from the connector portion 264. In some examples, the connector 206 includes more than one arm portion 272 such as two, three, four arm portions 272.

The connector portion 264 includes a distal end portion 205 and a proximal end portion 207. The distal end portion 205 of the connector portion 264 is configured to connect to the proximal end portion 203 of the sheath shaft 202, and the proximal end portion 207 of the connector portion 264 is configured to connect to the cap member 208. In some examples, the connector portion 264 is straight or linear and has a length that extends in the direction A1. The connector portion 264 includes a tubular member that defines a lumen 223 that extends between (and through) the distal end portion 205 and the proximal end portion 207. The connector portion 264 has a length in the direction A1 that extends from an opening 251 on the distal end portion 205 to an opening 253 on the proximal end portion 207.

In some examples, the distal end portion 205 includes a hub connector that is configured to mate with the hub connector 256 on the proximal end portion 203 of the sheath shaft 202. In some examples, the hub connector is a male hub connector. In some examples, the hub connector is a female hub connector. In some examples, the distal end portion 205 includes a tapered portion. In some examples, the distal end portion 205 of the connector portion 264 is permanently attached to the sheath shaft 202. In some examples, the distal end portion 205 is attached to the sheath shaft 202 using an adhesive material (e.g., glue). In some examples, the distal end portion 205 is releasably connected to the sheath shaft 202 (e.g., via hub connectors). A connector 206 that is releasably coupled to the sheath shaft 202 may allow the connector 206 to be connected after the sheath device 200 has been placed (delivered into the human body). When the connector portion 264 is coupled to the sheath shaft 202, the lumen 221 of the sheath shaft 202 and the lumen 223 of the connector portion 264 are aligned.

The proximal end portion 207 is configured to connect (e.g., removably connect) to the cap member 208. The connector portion 264 may include threads 280 on the proximal end portion 207 of the connector portion 264. In some examples, the threads 280 are external threads, e.g., threads defined on the outer surface of the connector portion 264 at the proximal end portion 207. The inner surface of the proximal end portion 207 may define a cavity 270 that is configured to house the elastic member 220. In some examples, the proximal end portion 207 has a size (e.g., an inner diameter and/or an outer diameter) that is larger than other portions of the connector portion 264 including the distal end portion 205. In some examples, the elastic member 220 is disposed entirely within the cavity 270 of the proximal end portion 207. In some examples, the elastic member 220 is disposed within the cavity 270 of the proximal end portion 207 and a portion of the cap member 208.

The elastic member 220 is configured to operate as a valve. The elastic member 220 defines a lumen 284 (e.g., through lumen) that is configured to be compressed to seal the valve (e.g., prevent the transfer of fluid/debris through the lumen 284) or lock onto one or more elongated devices (e.g., including the dilator 214). In some examples, the lumen 284 may have a round (e.g., circular) cross-sectional shape. In some examples, the lumen 284 may have other cross-sectional shapes such as oval, hexagon, polygon, square, or a star.

In some examples, the elastic member 220 is configured to operate in a range of positions between an open position (e.g., fully open position) and a closed position (e.g., fully sealed position). In the open position, elongated member(s) and/or fluid/debris may pass through the lumen 284 of the elastic member 220. In the closed position, the elongated member(s) and/or fluid/debris material is restricted or prevented from moving through the elastic member 220.

A distal end 291 of the elastic member 220 may contact a surface of the connector portion 264, and a proximal end 293 of the elastic member 220 may contact a surface of the cap member 208. The cavity 270 may confine the elastic member 220 from expanding outward as the lumen 284 is compressed by a plunger 286 of the cap member 208. The elastic member 220 is adjustable (e.g., by rotating the cap member 208) to a number of positions, e.g., to prevent the passage of fluid/debris through the elastic member 220 (e.g., through the connector portion 264) or to lock onto one or more elongated members, including the dilator 214, thereby preventing the elongated member(s) from moving within the sheath device 200. In some examples, the elastic member 220 may be closed (e.g., the lumen 284 is constricted) to drain the kidney (e.g., via the arm portion 272), to locked onto the dilator 214 (or other elongated devices), or to shut off access to the connector portion 264.

The threads 280 are configured to interact with threads 292 on the cap member 208 when the cap member 208 is rotated, thereby causing the cap member 208 to translate in the direction A1 (e.g., the distal direction). For example, the threads 280 and the threads 292 are configured to contact each other such that rotation of the cap member 208 in the direction of R causes cause a plunger 286 of the cap member 208 to compress the elastic member 220 in the direction A1 (e.g., the distal direction). Compression of the elastic member 220 causes the lumen 284 of the elastic member 220 to narrow, which, when sufficiently compressed, causes the elastic member 220 to seal or lock. Rotation of the cap member 208 in the direction of L causes the plunger 286 of the cap member 208 to move in the direction A1 (e.g., the proximal direction), which allows the lumen 284 of the elastic member 220 to self-expand or return to its normal state to unseal or to unlock.

The connector portion 264 includes one or more wings 216. A wing 216 may be a protrusion that extends (e.g., radially extends) from an outer surface of the connector portion 264. In some examples, the wings 216 may extend from a location on the outer surface between the threads 280 and the arm portion 272. The wings 216 may be used by an operator to facilitate the coupling of the cap member 208 to the connector portion 264. In some examples, the connector portion 264 includes two wings 216 that extend opposite to each other on the connector portion 264. In some examples, the connector portion 264 includes a single wing 216. In some examples, the connector portion 264 includes more than two wings 216.

The arm portion 272 may extend from a portion of the connector portion 264 between the distal end portion 205 of the connector portion 264 and the proximal end portion 207 of the connector portion 264. The arm portion 272 may include a tubular member that defines a lumen 227. The arm portion 272 may define an opening 255 to the lumen 227. The lumen 227 may be connected to and extend from the lumen 223 of the connector portion 264. When the valve (e.g., the elastic member 220) is closed and a negative pressure is applied to the opening on the arm portion 272, debris and/or fluid may flow from the body of the patient to the sheath shaft 202 and to (and out of) the arm portion 272. In some examples, the arm portion 272 may be disposed at an angle (e.g., non-zero, non-perpendicular angle) with respect to the connector portion 264. In some examples, the arm portion 272 includes a straight or linear portion that is angled from the connector portion 264 in the direction A1 (e.g., proximal direction) and the direction A2. In some examples, the arm portion 272 is configured to be coupled to another device. In some examples, the arm portion 272 may include threads 271 (e.g., a medical Luer threads (hub), a barb thread, or a screw thread, etc.) to enable the connection of a tube and/or connector for a negative pressure outlet or for a positive pressure fluid inlet.

In some examples, an elongated device (e.g., a smaller diameter) is configured to pass through the lumen 227 of the arm portion 272. In some examples, the arm portion 272 is configured to be coupled to a device that may deliver or generate negative pressure (e.g., a suctioning device). In some examples, as shown in FIG. 2E, the arm portion 272 includes a hole 273. In some examples, the hole 273 extends through the grip 274 and a sidewall of the arm portion 272. In some examples, the hole 273 is a negative pressure governing hole. Covering or partially covering the hole 273 with a finger may increase the negative pressure while uncovering the hole 273 may decrease the negative pressure through the lumen 221 of the sheath shaft 202. In some examples, when the hole 273 is not covered, the lumen 221 may be open to the atmosphere and is used as a drain to relieve fluid pressure.

In some examples, the arm portion 272 is used for the injection of a fluid between the outer diameter of an elongated device and an inner diameter of the sheath shaft 202. In some examples, the sheath device 200 includes a removable cover (not shown) that is configured to be placed over the hole 273 to close the hole 273. In some examples, the removable cover includes a thread cap, plug, or tape, etc. In some examples, the arm portion 272 does not include a hole. In some examples, the sheath assembly fluid inlet and outlet may be controlled by a fluid management system (FMS) and a sensor (e.g., a pressure and/or temperature sensor). In some examples, an elongated device may have at least one lumen that may be used as either an inlet lumen or an outlet to be the opposite flow direction of the sheath shaft 202 to have a continuous aspiration flow. For example, if the sheath device 200 has a negative pressure outlet flow, the elongated device may have a positive inlet flow.

The connector 206 includes one or more grips such as a grip 274 and a grip 260. The grip 274 may be connected to the connector portion 264 and the arm portion 272 and may extend along at least a portion of the length of the arm portion 272. In some examples, the grip 274 is coupled to an outer, distal surface of the arm portion 272. In some examples, the grip 274 includes a surface that includes one or more curved portions. The grip 260 may contact the connector portion 264 and protrude away (e.g., radially extend) from the connector portion 264. In some examples, the grip 274 and the grip 260 include portions that extend in opposite directions from each other. The grip 260 includes a surface that includes one or more curved portions. The connector 206 may also include a grip 277. The grip 277 may include a portion that is connected to the connector portion 264 and a portion that is connected to the arm portion 272. In some examples, the grip 277 is coupled to a portion of the arm portion 272 that is opposite to the grip 274. In some examples, the grip 277 is coupled to an outer, proximal surface of the arm portion 272. The grip 274, the grip 260, and/or the grip 277 may aid in holding the connector 206 to facilitate the rotational coupling of the cap member 208 to the proximal end portion 207 of the connector portion 264.

The cap member 208 is configured to be rotatably coupled to the proximal end portion 207 of the connector portion 264. In some examples, the cap member 208 includes a polymer-based material. In some examples, the cap member 208 includes a polycarbonate material. The cap member 208 includes a distal end portion 209 and a proximal end portion 211. The cap member 208 may include a tubular member. In some examples, the cap member 208 includes a cylindrical portion. The cap member 208 has a length that extends along the direction A1. In some examples, the cap member 208 includes an inner surface defining an inner diameter and an outer surface defining an outer surface. The cap member 208 includes a lumen 225 that extends between (and through) the distal end portion 209 and the proximal end portion 211. The cap member 208 includes a plunger 286 that is disposed within the cavity defined by the inner surface of the cap member 208. In some examples, the plunger 286 is coupled to the inner surface of the cap member 208. In some examples, the plunger 286 is a linear protrusion that extends in the cavity in the direction A1. In some examples, the plunger 286 includes an inner tubular member, and the inner tubular member defines the lumen 225. In some examples, when the cap member 208 is rotated on the connector portion 264, the lumen 225 aligns with the lumen 223.

The cap member 208 may define threads 292. In some examples, the threads 292 are defined on the distal end portion 209 of the cap member 208. In some examples, the threads 292 are internal threads. For example, the threads 292 may be defined on the inner surface of the tubular or cylindrical portion. The threads 280 on the connector portion 264 and the threads 292 are configured to contact each other such that rotation of the cap member 208 in the direction of R causes cause the plunger 286 of the cap member 208 to compress the elastic member 220 in the direction A1 (e.g., the distal direction).

Compression of the elastic member 220 causes the lumen 284 of the elastic member 220 to narrow, which, when sufficiently compressed, causes the elastic member 220 to seal or lock onto one or more elongated devices (including the dilator 214) that are disposed in the lumen 284 of the elastic member 220. Rotation of the cap member 208 in the direction of L causes the plunger 286 of the cap member 208 to move in the direction A1 (e.g., the proximal direction), which allows the lumen 284 of the elastic member 220 to self-expand or return to its normal state to unseal or to unlock. In other words, to seal, lock or close the valve, the cap member 208 is rotated in the direction of arrow R to translate rotational motion to linear motion in the distal direction along the direction A1. When the cap member 208 rotates, the elastic member 220 is compressed by the plunger 286, which causes the lumen 284 of the elastic member 220 to narrow. The elastic member 220 may be compressed linearly, radially, twisted or a combination thereof. In some examples, the connector 306 is configured to operate as a barrier or restriction to prevent the elastic member 220 from deforming outward such that the elastic deformation concentrates inward to narrow the lumen 284 of the elastic member 220. To allow the elastic member 220 to return to its normally opened state, the cap member 208 is rotated in the direction of arrow L to translate rotational motion to linear motion in the proximal direction along the direction A1 to allow the elastic member 220 to self-expand.

The cap member 208 may include one or more fins 218. In some examples, the fins 218 are defined on the distal end portion 209 of the cap member 208. A fin 218 may be a protrusion that extends (e.g., radially extends) from an outer surface of the tubular portion of the cap member 208. The fins 218 may be used by an operator to rotate the cap member 208. The fins 218 extend radially outward to allow positive/secure engagement of the thumb to rotate the cap member 208 in the direction of arrow R or in the direction of arrow L. In some examples, the cap member 208 includes four fins 218. However, the cap member 208 may include any number of fins 218 such as one, two, or three fins 218 or any number greater than four fins 218.

The cap member 208 may include a funnel 222. The funnel 222 may be defined on the proximal end portion 211 of the cap member 208. The funnel 222 may be a component that defines a cavity that increases in size towards the proximal end. In some examples, the funnel 222 may define a holder clip 296 configured to interact with one or more elongated members to hold the elongated member(s) in place. In some examples, the holder clip 296 includes a protrusion that extends from the funnel 222. In some examples, the holder clip 296 includes an L-shaped protrusion. In some examples, the holder clip 296 is configured to engage with a component (e.g., a protrusion) on a hub connector 224 on the dilator 214 to secure the dilator 214.

In some examples, the connector 206 and the cap member 208 are configured to be operated using a single hand while the other hand is used to operate the control handle of the scope or another elongated device. Both hands, if available, may be used to operate the connector 206 and the cap member 208 to open or close the elastic member 220. In some examples, a portion of the first hand is used to secure the connector 206 (e.g., prevent the connector 206 from rotating) while the thumb is used to rotate the cap member 208. In some examples, the index finger of the first hand is also used to aid in rotating the cap member 208. In some examples, securing (e.g., locking) the sheath device 200 to a steerable device shaft (e.g., dilator 214) allows for both devices to move in unison by moving either the dilator 214 or the sheath device 200. The lock may also maintain the extended or retracted position of the sheath device 200 relative to the steerable device.

Referring to FIG. 2H, in some examples, the index finger 231 may contact the grip 277 and rest on the wings 216. The middle finger 241 may contact the grip 260 to prevent the connector 206 from rotating. The thumb 233 (and the index finger 231) may be used to rotate the cap member 208. In some examples, referring to FIG. 2I, the index finger 231 may be inserted between the connector portion 264 and the arm portion 272 and the rest of the fingers may grip the distal portion of the connector 206 and sheath shaft 202 to prevent the connector 206 from rotating while the thumb 235 is used to rotate the cap member 208. In some examples, referring to FIG. 2J, the index finger 231 is positioned on the wing 216 while the thumb 235 is positioned on a fin 218. Squeezing the wing 216 and the fin 218 towards each other may result in rotating the cap member 208 in the direction of arrow R. In some examples, the thumb 235 and the index finger 231 is switched (not shown), where the index finger 231 is positioned on the fin 218 while the thumb 235 is positioned on the wing 216. Squeezing the wing 216 and the fin 218 towards each other may result in rotating the cap member 208 in the direction of arrow L. Multiple rotations may be needed to fully close or open the valve. In some examples, the screw thread may be a coarse thread such that the elastic member 220 is opened and closed by a single or a fraction of a revolution. In some examples, the threads may be replaced by a pin on the connector 206 that follows a helical slot on the cap member 208. In some examples, the elastic member 220 is opened and closed by pushing the cap member 208 inward and outward.

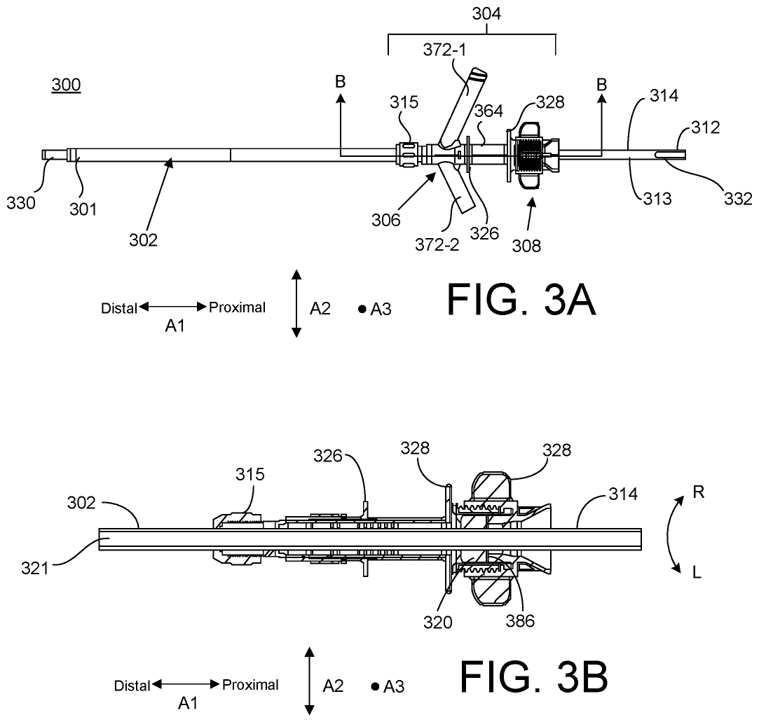

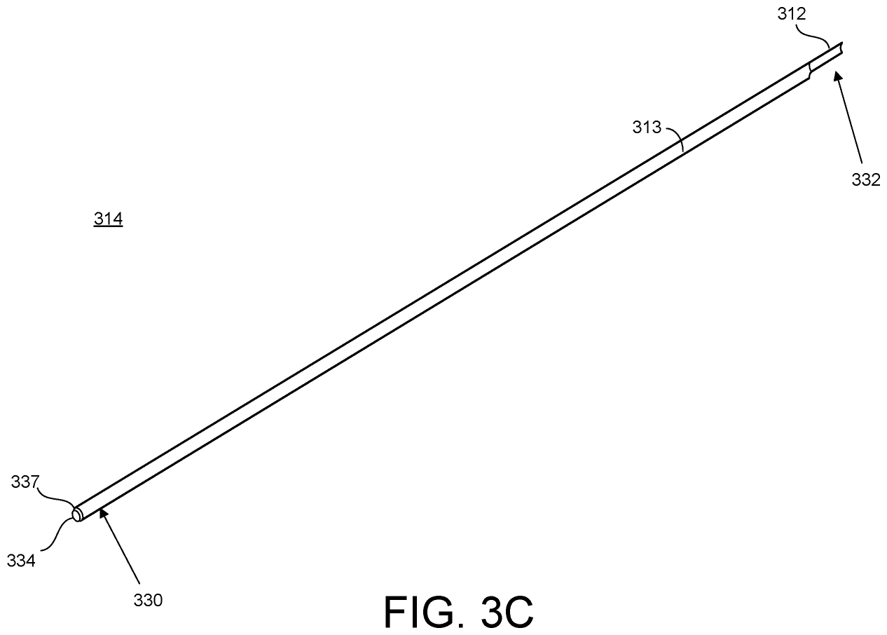

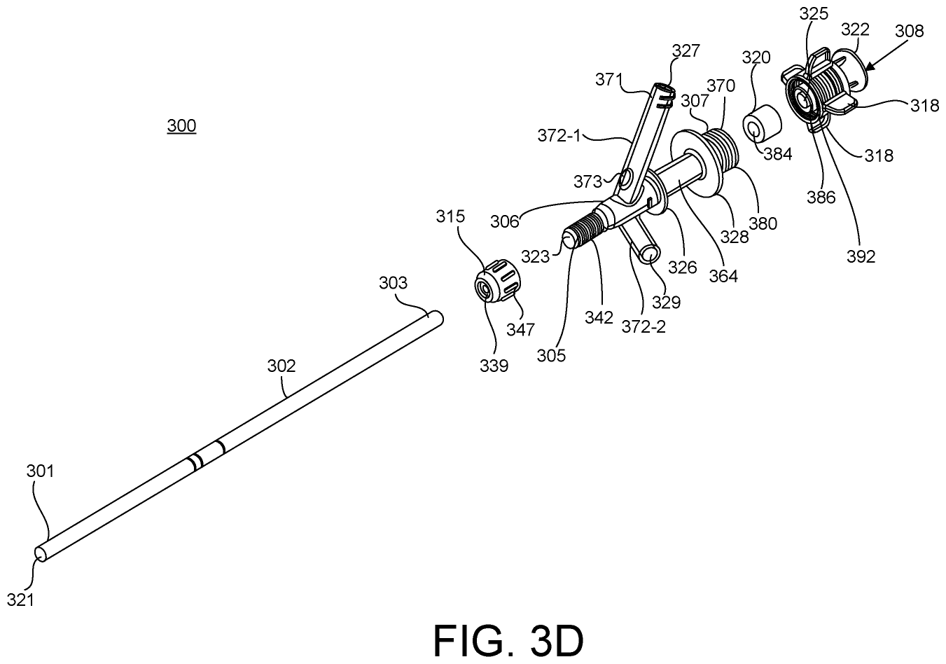

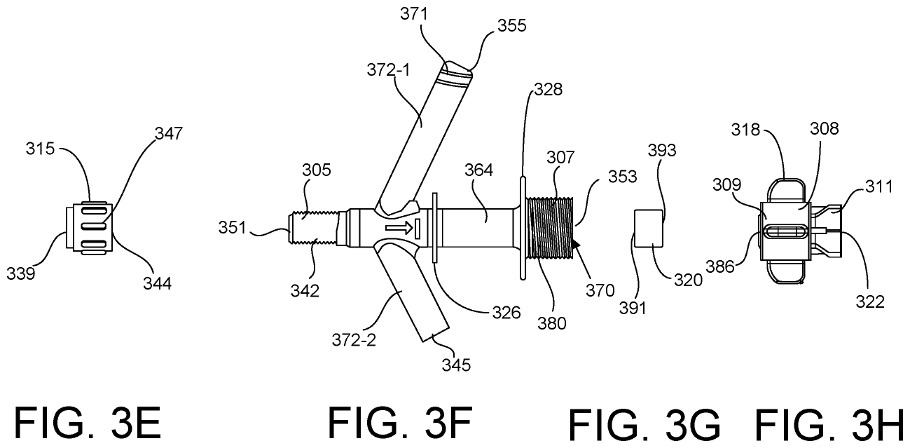

FIGS. 3A through 3H illustrate an example of a sheath device 300 according to another aspect. The sheath device 300 may be an example of any of the sheath devices discussed herein and may include any of the details discussed with reference to the other figures. The sheath device 300 is configured to facilitate the removal of stones, stone fragments, or other debris from the human body (e.g., the kidney, urinary tract, etc.). In some examples, the sheath device 300 may be configured to be used with a spacer 314 to place a portion of the sheath device 300 into the human body (e.g., the kidney, urinary tract). In some examples, the spacer 314 is configured to receive a steerable device shaft.

FIG. 3A illustrates a side perspective of the sheath device 300 with the spacer 314 disposed within the lumen of the sheath device 300. FIG. 3B illustrates a cross-sectional view of the sheath device 300 taken along a line B-B. FIG. 3C illustrates an example of the spacer 314 that is used to insert a portion of the sheath device 300 into the body of a patient. The sheath device 300 includes a sheath shaft 302 and a valve assembly 304. The valve assembly 304 includes a compression cap 315, a connector 306, an elastic member 320 (e.g., a valve), and a cap member 308. FIG. 3D illustrates an example of the sheath shaft 302, the compression cap 315, the connector 306, the elastic member 320, and the cap member 308. FIG. 3E illustrates an example of the compression cap 315. FIG. 3F illustrates an example of the connector 306. FIG. 3G illustrates an example of the elastic member 320 configured to operate as a valve. FIG. 3H illustrates an example of the cap member 308.

In some examples, the sheath shaft 302 may be delivered over a steerable device shaft (not shown) using a spacer 314. The spacer 314 may be an example of an elongated member. A lumen 334 of the spacer 314 may have a size (e.g., a diameter) that is equal to or greater than the outer diameter of the steerable device shaft. The spacer 314 may include a circular cross-section. In some examples, the spacer 314 includes a tapered distal portion 337, a proximal tab 312, and a slit 313 that extends the length of the spacer 314. In some examples, the steerable device shaft includes a dilator (e.g., the dilator 214 of FIGS. 2A through 2J). The spacer 314 is inserted into the sheath device 300 and locked together with the valve assembly 304. A steerable device is then inserted into the lumen 334 of the spacer 314 and the entire assembly may be inserted over a guidewire with the steerable device tip leading the way. Once in the appropriate location in the body of the patient, in some examples, the sheath shaft 302 is moved over the steerable device shaft. In some examples, the valve is unlocked (e.g., the elastic member 320 is uncompressed), and the proximal tab 312 may be pull in a proximal direction proximal and away from the steerable device and out of the sheath shaft 302 to allow the slit 313 of the spacer 314 to widen and separated, thereby leaving the steerable device and the sheath device 300 in place for use.

The sheath shaft 302 may be similar to the sheath shaft 202 of FIGS. 2A through 2J, and, therefore, may include any of the details discussed with reference to FIGS. 2A through 2J. For example, the sheath shaft 302 includes a distal end portion 301 and a proximal end portion 303. The sheath shaft 302 defines a lumen 321 that extends between (and through) the distal end portion 301 and the proximal end portion 303. The proximal end portion 303 is configured to connect to a distal end portion 305 of the connector 306 of the valve assembly 304.

In some examples, the proximal end portion 303 includes a flared portion (e.g., an enlarged portion). The proximal end portion 303 is configured to be disposed over the compression cap 315. In other words, at least a portion of the compression cap 315 is inserted into the lumen 321 of the sheath shaft 302 at the proximal end portion 303. The compression cap 315 may include a cylindrical portion. The compression cap 315 may include an outer surface defining an outer diameter and an inner surface defining an inner diameter and a lumen that extends between an opening 339 and an opening 344. In some examples, the compression cap 315 includes surface features 347 configured to contact the inner surface of the sheath shaft 302. The surface features 347 may be defined on the outer surface of the compression cap 315. In some examples, the surface features 347 include protrusions that are aligned around the outer surface. The compression cap 315 is configured to be removable coupled to the distal end portion 305 of the connector 306. For example, the compression cap 315 may include internal threads (not shown) on the inner surface of the cylindrical portion. The internal threads are configured to engage threads 342 on the distal end portion 305 of the connector 306.

The sheath shaft 302 may include a tubular member. In some examples, the sheath shaft 302 includes a circular profile. The sheath shaft 302 may include an inner surface defining an inner diameter (and a lumen 321) and an outer surface defining an outer diameter. The sheath shaft 302 has a length that extends in a direction A1. The direction A1 may be referred to as a distal direction or a proximal direction. A direction A3 is perpendicular to the direction A1. A direction A3 (represented by a black dot) extends into (and out of) the page and is orthogonal to directions A1 and A3. The sheath shaft 302 may be constructed from one or more polymer materials (including laser-resistant materials), as discussed with reference to the sheath device 200 of FIGS. 2A through 2J.

As shown in FIG. 3C, the spacer 314 includes a distal end portion 330 and a proximal end portion 332. In some examples, the distal end portion 330 includes a tapered distal portion 337. The spacer 314 may have an inner surface that defines an inner diameter and an outer surface that defines an outer diameter. In some examples, the spacer 314 defines a lumen 334. The lumen 334 of the spacer 314 may have a size (e.g., a diameter) that is equal to or greater than the outer diameter of the steerable device shaft. The spacer 314 may have a circular cross-section. In some examples, the spacer 314 includes a tapered distal portion 337, a proximal tab 312, and a slit 313 that extends the length of the spacer 314.

The valve assembly 304 may include a connector 306, a cap member 308, and an elastic member 320 configured to operate as a valve that opens and closes. As indicated above, in some examples, the proximal end portion 303 of the sheath shaft 302 is coupled to the compression cap 315 and the compression cap 315 is screwed onto the distal end portion 305 of the connector 306 The elastic member 320 may be disposed within a cavity 370 of the connector 306. In some examples, the cap member 308 is rotatably coupled to the connector 306 (e.g., screwed on the connector 306). By moving (e.g., rotating) the cap member 308, the cap member 308 may compress or expand the elastic member 320, thereby closing or opening the valve.

The connector 306 may include a connector portion 364, an arm portion 372-1, and an arm portion 372-2. The arm portion 372-1 may branch (e.g., extend) from the connector portion 364. The arm portion 372-2 may branch (e.g., extend) from the connector portion 364.

The connector portion 364 includes a distal end portion 305 and a proximal end portion 307. The distal end portion 305 of the connector portion 364 is configured to connect to the compression cap 315. In some examples, the distal end portion 305 includes threads 342 (e.g., external threads) on an outer surface of the connector portion 364. The compression cap 315 (and the sheath shaft 302) may be screwed onto the threads 342 on the distal end portion 305. In some examples, the connector portion 364 is straight or linear and has a length that extends in the direction A1. The connector portion 364 includes a tubular member that defines a lumen 323 that extends between (and through) the distal end portion 305 and the proximal end portion 307. The connector portion 364 has a length in the direction A1 that extends from an opening 351 on the distal end portion 305 to an opening 353 on the proximal end portion 307. When the connector portion 364 is coupled to the sheath shaft 302, the lumen 321 of the sheath shaft 302 and the lumen 323 of the connector portion 364 are aligned.

The proximal end portion 307 is configured to connect (e.g., removably connect) to the cap member 308. The connector portion 364 may include threads 380 on the proximal end portion 307 of the connector portion 364. In some examples, the threads 380 are external threads, e.g., threads defined on the outer surface of the connector portion 364 at the proximal end portion 307. The inner surface of the proximal end portion 307 may define a cavity 370 that is configured to house the elastic member 320. In some examples, the proximal end portion 307 has a size (e.g., an inner diameter and/or an outer diameter) that is larger than other portions of the connector portion 364 including the distal end portion 305. In some examples, the elastic member 320 is disposed entirely within the cavity 370 of the proximal end portion 307. In some examples, the elastic member 320 is disposed within the cavity 370 of the proximal end portion 307 and a portion of the cap member 308.

The elastic member 320 is configured to operate as a valve. The elastic member 320 defines a lumen 384 (e.g., a through lumen) that is configured to be compressed to seal the valve (e.g., prevent the transfer of fluid/debris through the lumen 384) or lock onto one or more elongated devices (e.g., including the spacer 314). In some examples, the lumen 384 may have a round (e.g., circular) cross-sectional shape. In some examples, the lumen 384 may have other cross-sectional shapes such as oval, hexagon, polygon, square, or a star.

A distal end 391 of the elastic member 320 may contact a surface of the connector portion 364, and a proximal end 393 of the elastic member 320 may contact a surface of the cap member 308. The cavity 370 may confine the elastic member 320 from expanding outward as the lumen 384 is compressed by a plunger 386 of the cap member 308. The elastic member 320 is adjustable (e.g., by rotating the cap member 308) to a number of positions, e.g., to prevent the passage of fluid/debris through the elastic member 320 (e.g., through the connector portion 364) or to lock onto one or more elongated members, including the spacer 314, thereby preventing the elongated member(s) from moving within the sheath device 300. In some examples, the elastic member 320 may be closed (e.g., the lumen 384 is constricted) to drain the kidney (e.g., via the arm portion 372-1 or the arm portion 372-2), to lock onto the spacer 314 (or other elongated devices), or to shut off access to the connector portion 364.

The threads 380 are configured to interact with threads 392 on the cap member 308 when the cap member 308 is rotated, thereby causing the cap member 308 to translate in the direction A1 (e.g., the distal direction). For example, the threads 380 and the threads 392 are configured to contact each other such that rotation of the cap member 308 in the direction of R causes cause a plunger 386 of the cap member 308 to compress the elastic member 320 in the direction A1 (e.g., the distal direction). Compression of the elastic member 320 causes the lumen 384 of the elastic member 320 to narrow, which, when sufficiently compressed, causes the elastic member 320 to seal or lock. Rotation of the cap member 308 in the direction of L causes the plunger 386 of the cap member 308 to move in the direction A1 (e.g., the proximal direction), which allows the lumen 384 of the elastic member 320 to self-expand or return to its normal state to unseal or to unlock.

The connector portion 364 may include a ring member 326 and a ring member 328. The ring member 326 may extend around a circumference of the outer surface and extend radially outward at a first location (in the direction A1) on the connector portion 364. In some examples, the first location is proximal to the arm portion 372-1 and the arm portion 372-2. In some examples, the first location is distal to the ring member 328. The ring member 328 may extend around a circumference of the outer surface and extend radially outward at a second location (in the direction A1) on the connector portion 364. In some examples, the second location is proximal to the ring member 326 and distal to the threads 380. In some examples, the ring member 328 has a size that is larger than the size of the ring member 326. To operate the valve with a single hand, in some examples, the connector portion 364 is placed between index finger and the middle finger and between the ring member 326 and the ring member 328 to hold the connector 306 while the thumb of the same hand operates (rotates) the cap member 308.

The arm portion 372-1 may extend from a portion of the connector portion 364 between the distal end portion 305 of the connector portion 364 and the proximal end portion 307 of the connector portion 364. The arm portion 372-1 may include a tubular member that defines a lumen 327. The arm portion 372-1 may define an opening 355 to the lumen 327. The lumen 327 may be connected to and extend from the lumen 323 of the connector portion 364. When the valve (e.g., the elastic member 320) is closed and a negative (or positive) pressure is applied to the opening 355 on the arm portion 372-1, debris and/or fluid may flow from the body of the patient to the sheath shaft 302 and to (and out of) the arm portion 372-1 (or vice versa to insert a fluid into the body). In some examples, the arm portion 372-1 may be disposed at an angle (e.g., non-zero, non-perpendicular angle) with respect to the connector portion 364. In some examples, the arm portion 372-1 includes a straight or linear portion that is angled from the connector portion 364 in the direction A1 (e.g., proximal direction) and the direction A2. In some examples, the arm portion 372-1 is configured to be coupled to another device. In some examples, the arm portion 372-1 may include threads 371 (e.g., a medical Luer threads (hub), a barb thread, or a screw thread, etc.) to enable the connection of a tube and/or connector for a negative pressure outlet or for a positive pressure fluid inlet.

In some examples, the arm portion 372-1 includes a hole 373. In some examples, the hole 373 extends through a sidewall of the arm portion 372-1. In some examples, the hole 373 is a negative pressure governing hole. Covering or partially covering the hole 373 with a finger may increase the negative pressure while uncovering the hole 373 may decrease the negative pressure through the lumen 321 of the sheath shaft 302.

The arm portion 372-2 may extend from a portion of the connector portion 364 between the distal end portion 305 of the connector portion 364 and the proximal end portion 307 of the connector portion 364. In some examples, the arm portion 372-2 has a length that is less than the length of the arm portion 372-1. The arm portion 372-2 may include a tubular member that defines a lumen 329. The arm portion 372-2 may define an opening 345 to the lumen 329. The lumen 329 may be connected to and extend from the lumen 323 of the connector portion 364. When the valve (e.g., the elastic member 320) is closed and a negative (or positive) pressure is applied to the opening 345 on the arm portion 372-1, debris and/or fluid may flow from the body of the patient to the sheath shaft 302 and to (and out of) the arm portion 372-2 (or vice versa to insert a fluid into the body). In some examples, the arm portion 372-2 may be disposed at an angle (e.g., non-zero, non-perpendicular angle) with respect to the connector portion 364. In some examples, the arm portion 372-2 includes a straight or linear portion that is angled from the connector portion 364 in the direction A1 (e.g., proximal direction) and the direction A2. In some examples, the arm portion 372-2 is configured to be coupled to another device.

The cap member 308 is configured to be rotatably coupled to the proximal end portion 307 of the connector portion 364. In some examples, the cap member 308 includes a polymer-based material. In some examples, the cap member 308 includes a polycarbonate material. The cap member 308 includes a distal end portion 309 and a proximal end portion 311. The cap member 308 may include a tubular member. In some examples, the cap member 308 includes a cylindrical portion. The cap member 308 has a length that extends along the direction A1. In some examples, the cap member 308 includes an inner surface defining an inner diameter and an outer surface defining an outer surface. The cap member 308 includes a lumen 325 that extends between (and through) the distal end portion 309 and the proximal end portion 311. The cap member 308 includes a plunger 386 that is disposed within the cavity defined by the inner surface of the cap member 308. In some examples, the plunger 386 is coupled to the inner surface of the cap member 308. In some examples, the plunger 386 is a linear protrusion that extends in the cavity in the direction A1. In some examples, the plunger 386 includes an inner tubular member, and the inner tubular member defines the lumen 325. In some examples, when the cap member 308 is rotated on the connector portion 364, the lumen 325 aligns with the lumen 323.

The cap member 308 may define threads 392. In some examples, the threads 392 are defined on the distal end portion 309 of the cap member 308. In some examples, the threads 392 are internal threads. For example, the threads 392 may be defined on the inner surface of the tubular or cylindrical portion.

The threads 380 on the connector portion 364 and the threads 392 are configured to contact each other such that rotation of the cap member 308 in the direction of R causes cause the plunger 386 of the cap member 308 to compress the elastic member 320 in the direction A1 (e.g., the distal direction). Compression of the elastic member 320 causes the lumen 384 of the elastic member 320 to narrow, which, when sufficiently compressed, causes the elastic member 320 to seal or lock. Rotation of the cap member 308 in the direction of L causes the plunger 386 of the cap member 308 to move in the direction A1 (e.g., the proximal direction), which allows the lumen 384 of the elastic member 320 to self-expand or return to its normal state to unseal or to unlock. In other words, to seal, lock or close the valve, the cap member 308 is rotated in the direction of arrow R to translate rotational motion to linear motion in the distal direction along the direction A1. When the cap member 308 rotates, the elastic member 320 is compressed by the plunger 386, which causes the lumen 384 of the elastic member 320 to narrow. To allow the elastic member 320 to return to its normally opened state, the cap member 308 is rotated in the direction of arrow L to translate rotational motion to linear motion in the proximal direction along the direction A1 to allow the elastic member 320 to self-expand.

The cap member 308 may include one or more fins 318. In some examples, the fins 318 are defined on the distal end portion 309 of the cap member 308. A fin 318 may be a protrusion that extends (e.g., radially extends) from an outer surface of the tubular portion of the cap member 308. The fins 318 may be used by an operator to rotate the cap member 308. The fins 318 extend radially outward to allow positive/secure engagement of the thumb to rotate the cap member 308 in the direction of arrow R or in the direction of arrow L. In some examples, the cap member 308 includes four fins 318. However, the cap member 308 may include any number of fins 318 such as one, two, or three fins 318 or any number greater than four fins 318. The cap member 308 may include a funnel 322. The funnel 322 may be defined on the proximal end portion 311 of the cap member 308. The funnel 322 may be a component that defines a cavity that increases in size towards the proximal end.

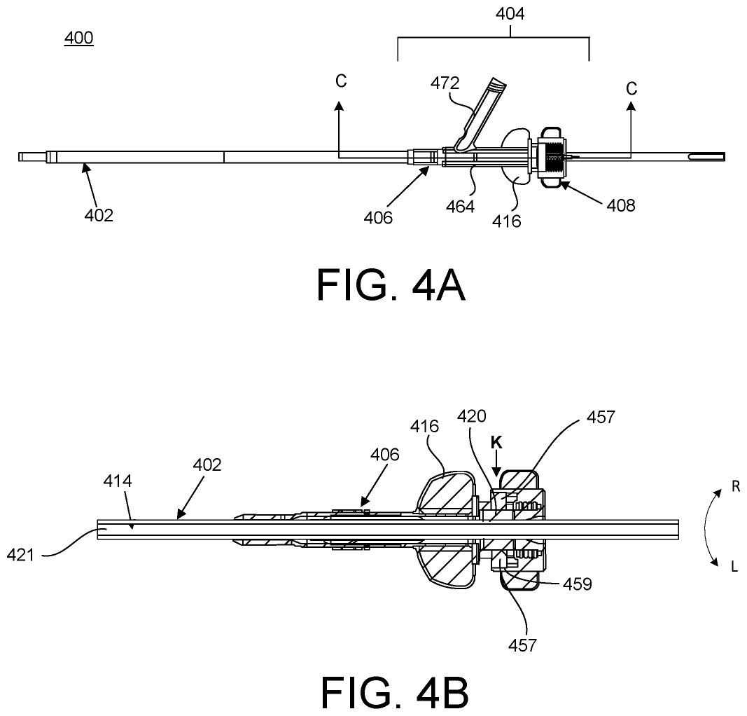

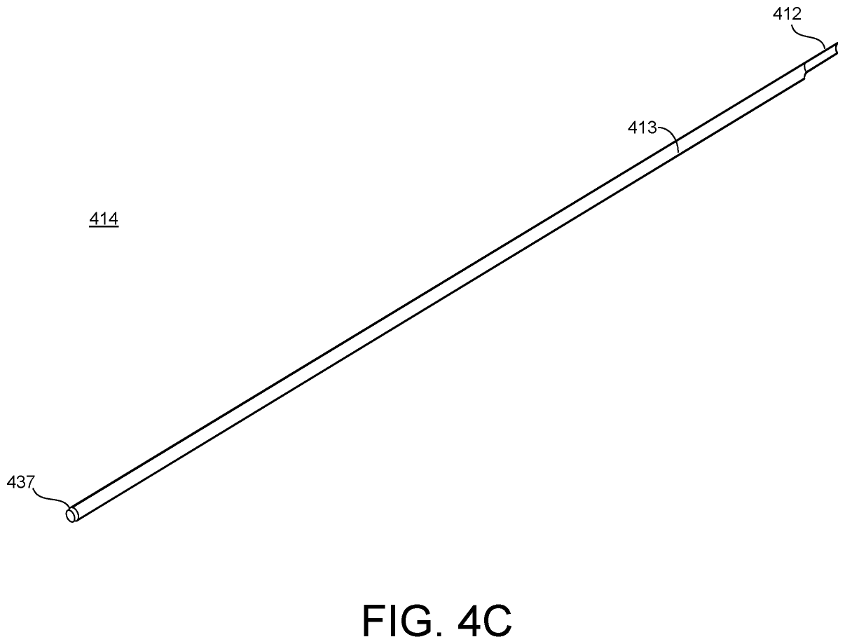

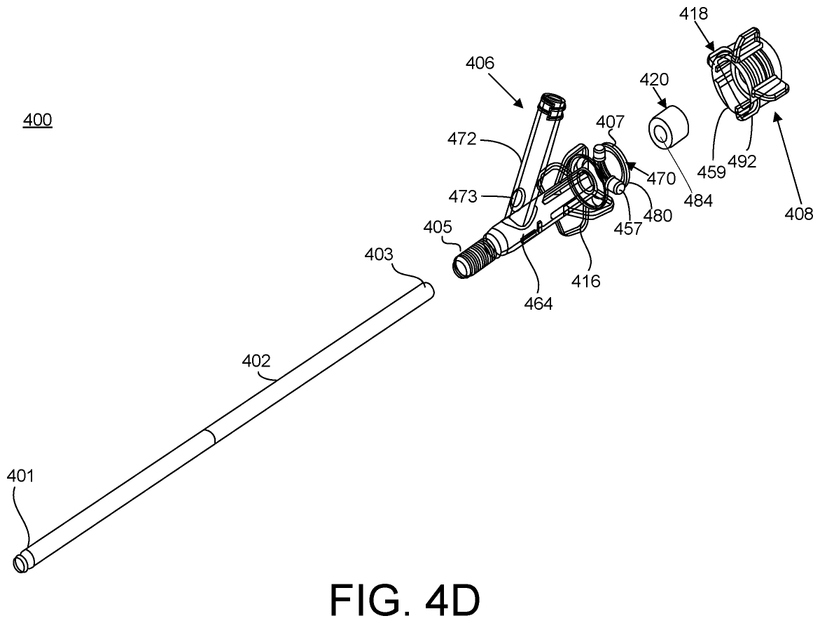

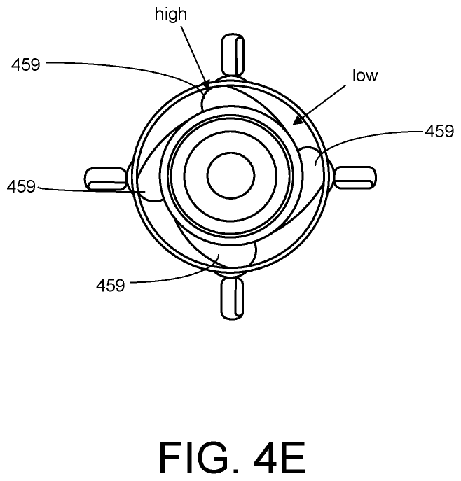

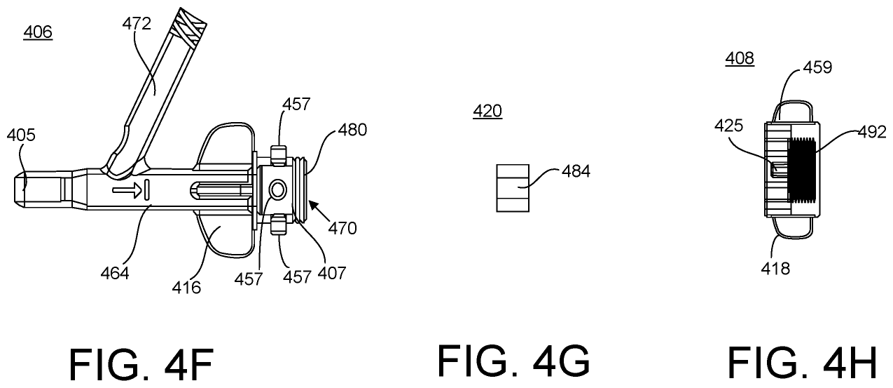

FIGS. 4A through 4H illustrate an example of a sheath device 400 according to another aspect. The sheath device 400 may be an example of any of the sheath devices discussed herein and may include any of the details discussed with reference to the other figures. The sheath device 400 is configured to facilitate the removal of stones, stone fragments, or other debris from the human body (e.g., the kidney, urinary tract, etc.). In some examples, the sheath device 400 may be configured to be used with a spacer 414 to place a portion of the sheath device 400 into the human body (e.g., the kidney, urinary tract). In some examples, the spacer 414 is configured to receive a steerable device shaft.

FIG. 4A illustrates a side perspective of the sheath device 400 with the spacer 414 disposed within the lumen of the sheath device 400. FIG. 4B illustrates a cross-sectional view of the sheath device 400 taken along a line C-C. FIG. 4C illustrates an example of the spacer 414 that is used to insert a portion of the sheath device 400 into the body of a patient. The sheath device 400 includes a sheath shaft 402 and a valve assembly 404. The valve assembly 404 includes a connector 406, an elastic member 420 (e.g., a valve), and a cap member 408. FIG. 4D illustrates an example of the sheath shaft 402, the connector 406, the elastic member 420, and the cap member 408. FIG. 4E illustrates a plurality of cams 459 that are configured on the cap member 408. FIG. 4F illustrates an example of the connector 406. FIG. 4G illustrates an example of the elastic member 420 configured to operate as a valve. FIG. 4H illustrates an example of the cap member 408.

In some examples, the elastic member 420 is compressed by one or more cams 459 disposed on the cap member 408 and one or more cam plungers 457 disposed on the connector 406. Although the figures depict four cams 459 and four cam plungers 457, the sheath device 400 may include any number of sets including one, two, three, or more than four. The cam(s) 459 on the cap member 408 may transfer the rotational motion of the cap member 408 to a radial motion K toward the elastic member 420. When a cam plunger 457 is engaged with the high point of a respective cam 459, the lumen 484 of the elastic member 420 is opened. When the cam plunger 457 is engaged with the low point of a respective cam 459, the lumen 484 of the elastic member 420. Rotation of the cap member 408 in the direction of arrow R or to the lower point of the cam 459 may close the valve. In a screw thread engagement between the cap member 408 and connector 406 (e.g., as shown in FIGS. 4A through 4H), the cap member 408 may be rotated in direction of arrow L to open the elastic.

The sheath shaft 402 may be similar to the sheath shaft 202 of FIGS. 2A through 2J, and, therefore, may include any of the details discussed with reference to FIGS. 2A through 2J. For example, the sheath shaft 402 includes a distal end portion 401 and a proximal end portion 403. The sheath shaft 402 defines a lumen 421 that extends between (and through) the distal end portion 401 and the proximal end portion 403. The proximal end portion 403 is configured to connect to a distal end portion 405 of the connector 406 of the valve assembly 404. In some examples, the distal end portion 405 is directly attached to the sheath shaft 402. The spacer 414 may be similar to the sheath shaft 302 of FIGS. 3A through 3H and may include any of the details discussed with reference to those figures. For example, the spacer 414 includes a tapered distal portion 437, a proximal tab 412, and a slit 413 that extends the length of the spacer 414.

The valve assembly 404 may include a connector 406, a cap member 408, and an elastic member 420 configured to operate as a valve that opens and closes. The elastic member 420 may be disposed within a cavity 470 of the connector 406. In some examples, the cap member 408 is rotatably coupled to the connector 406 (e.g., screwed on the connector 406).

The connector 406 may include a connector portion 464 and an arm portion 472. The connector portion 464 includes a distal end portion 405 and a proximal end portion 407. The distal end portion 405 of the connector portion 464 is configured to connect to the proximal end portion 403 of the sheath shaft 402. In some examples, the connector portion 464 is straight or linear and has a length that extends in the direction A1. The proximal end portion 407 is configured to connect (e.g., removably connect) to the cap member 408. The connector portion 464 may include threads 480 on the proximal end portion 407 of the connector portion 464. In some examples, the threads 480 are external threads, e.g., threads defined on the outer surface of the connector portion 464 at the proximal end portion 407. The inner surface of the proximal end portion 407 may define a cavity 470 that is configured to house the elastic member 420.

The connector portion 464 includes one or more wings 416. A wing 416 may be a protrusion that extends (e.g., radially extends) from an outer surface of the connector portion 464. In some examples, the wings 416 may extend from a location on the outer surface between the cam plungers 457 and the arm portion 472. The wings 416 may be used by an operator to facilitate the coupling of the cap member 408 to the connector portion 464. In some examples, the connector portion 464 includes four wings 416 that extend around the connector portion 464.

In some examples, the arm portion 472 includes a hole 473. In some examples, the hole 473 extends through a sidewall of the arm portion 472. In some examples, the hole 473 is a negative pressure governing hole. Covering or partially covering the hole 473 with a finger may increase the negative pressure while uncovering the hole 473 may decrease the negative pressure through the lumen 421 of the sheath shaft 402.

The cap member 408 may include a tubular member. In some examples, the cap member 408 includes a cylindrical portion. In some examples, the cap member 408 includes an inner surface defining an inner diameter and an outer surface defining an outer surface. The cap member 408 includes a lumen 425. The cap member 408 may include one or more cams 459. In some examples, a cam 459 is a protrusion (e.g., a sloped protrusion). As shown in FIG. 4E, in some examples, the cap member 408 includes four cams 459. However, the cap member 408 may include any number of cams 459 such as one, two, three, or any number greater than four. The connector portion 464 includes one or more cam plungers 457. In some examples, the cam plungers 457 are connected to and extend from the inner surface of the proximal end portion 407 of the connector portion 464 (e.g., extend into the cavity 470).

The elastic member 420 may be compressed by the cams 459 and the plungers 457 when the cap member 408 is rotated. The cam(s) 459 on the cap member 408 may transfer the rotational motion of the cap member 408 to a radial motion K (FIG. 4B) toward the elastic member 420. When a cam plunger 457 is engaged with the high point of a respective cam 459, the lumen 484 of the elastic member 420 is opened. When the cam plunger 457 is engaged with the low point of a respective cam 459, the lumen 484 of the elastic member 420. Rotation of the cap member 408 in the direction of arrow R or to the lower point of the cam 459 may close the valve. In a screw thread engagement between the cap member 408 and connector 406 (e.g., as shown in FIGS. 4A through 4H), the cap member 408 may be rotated in direction of arrow L to open the elastic.

The cap member 408 may define threads 492. In some examples, the threads 492 are defined on the distal end portion of the cap member 408. In some examples, the threads 492 are internal threads. For example, the threads 492 may be defined on the inner surface of the tubular or cylindrical portion. The cap member 408 may include one or more fins 418. In some examples, the fins 418 are defined on the distal end portion of the cap member 408. A fin 418 may be a protrusion that extends (e.g., radially extends) from an outer surface of the tubular portion of the cap member 408. The fins 418 may be used by an operator to rotate the cap member 408. The fins 418 extend radially outward to allow positive/secure engagement of the thumb to rotate the cap member 408 in the direction of arrow R or in the direction of arrow L. In some examples, the cap member 408 includes four fins 418. However, the cap member 408 may include any number of fins 418 such as one, two, or three fins 418 or any number greater than four fins 418.

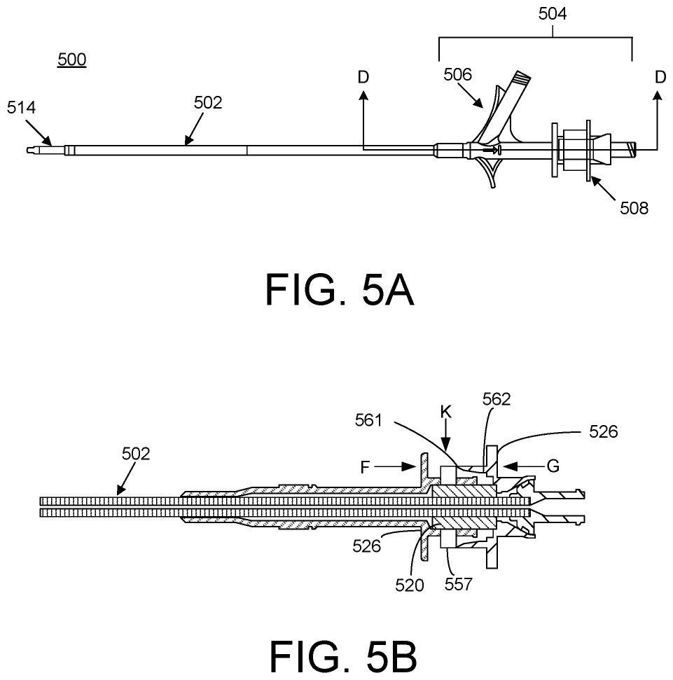

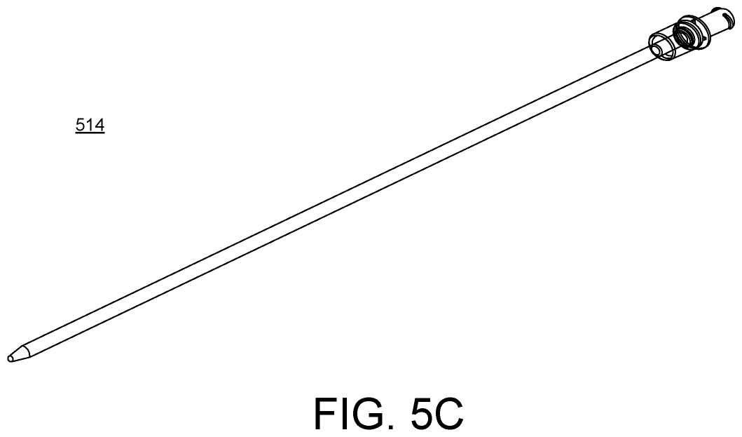

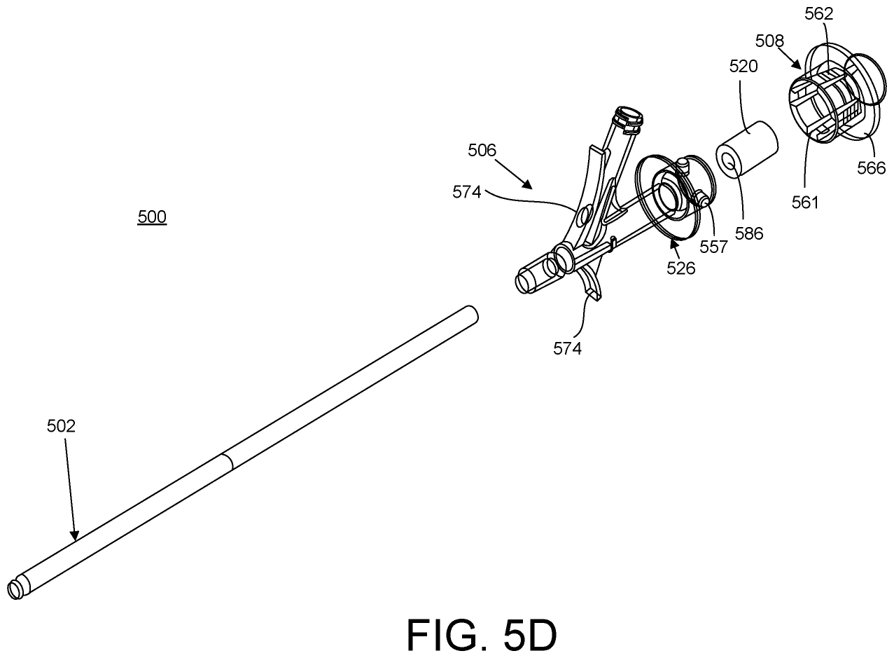

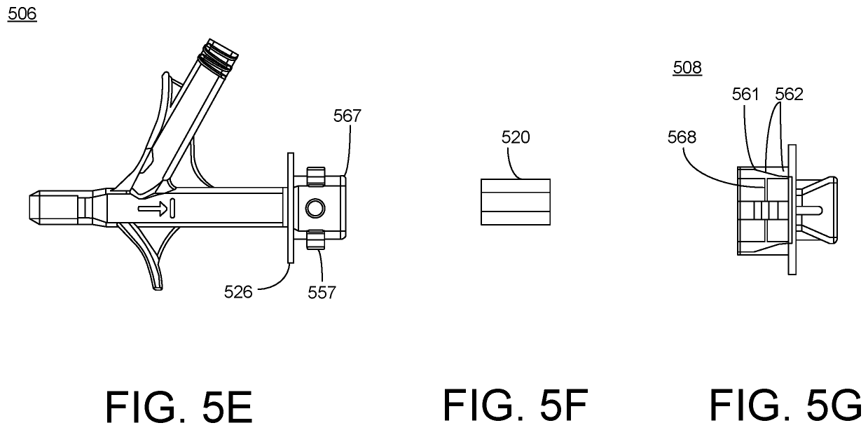

FIGS. 5A through 5G illustrate an example of a sheath device 500 according to another aspect. The sheath device 500 may be an example of any of the sheath devices discussed herein and may include any of the details discussed with reference to the other figures. The sheath device 500 is configured to facilitate the removal of stones, stone fragments, or other debris from the human body (e.g., the kidney, urinary tract, etc.). In some examples, the sheath device 500 may be configured to be used with a dilator 514 to place a portion of the sheath device 500 into the human body (e.g., the kidney, urinary tract). The dilator 514 may be similar to the dilator 214 of FIGS. 2A through 2J and may include any of the details discussed with reference to those figures.

FIG. 5A illustrates a side perspective of the sheath device 500 with the dilator 514 disposed within the lumen of the sheath device 500. FIG. 5B illustrates a cross-sectional view of the sheath device 500 taken along a line D-D. FIG. 5C illustrates an example of the dilator 514 that is used to insert a portion of the sheath device 500 into the body of a patient. The sheath device 500 includes a sheath shaft 502 and a valve assembly 504. The valve assembly 504 includes a connector 506, an elastic member 520 (e.g., a valve), and a cap member 508. FIG. 5D illustrates an example of the sheath shaft 502, the connector 506, the elastic member 520, and the cap member 508. FIG. 5E illustrates an example of the connector 506. FIG. 5F illustrates an example of the elastic member 520 configured to operate as a valve. FIG. 5G illustrates an example of the cap member 508.

The connector 506 may include a snap ramp 567, and the cap member 508 may include a snap hook 568. In some examples, the connector 506 and the cap member 508 may be coupled together (e.g., snapped together) by the snap ramp 567 on the connector 506 and the snap hook 568 on the cap member 508. The elastic member 520 may be compressed (e.g., a lumen 586 is narrowed) by one or more step ramps 561 on the connector 506 and one or more cam plungers 557. The step ramp 561 may transfer the linear motion (e.g., arrow G in FIG. 5B) of the cap member 508 to a radial motion K (FIG. 5B), towards the elastic member 520. As the cap member 508 is advanced in the direction of arrow G, the incline of the step ramp 561 may depress the cam plunger 557 to compress the elastic member 520. As the cap member 508 is retracted in the direction of arrow F (FIG. 5B), the elastic member 520 may self-expand and return the cam plunger 557 radially outward. The steps of the step ramp 561 may have flats 562 (e.g., dimples) in which hold the cam plunger 557 in position. At one flat 562 (e.g., a high flat), the elastic member 520 is opened. At another flat 562 (e.g., a middle flat), the elastic member 520 may be locked to a shaft (e.g., the dilator 514). At another flat 562 (e.g., a low flat), the elastic member may be closed. The valve may be closed with a single hand by squeezing a ring member 566 on the cap member 508 and a ring member 526 on the connector 506 together or by squeezing grip(s) 574 and the ring member 566 together. In some examples, the value may be opened by holding onto the connector 506 and with the thumb of the same hand to push the ring member 566 in the proximal direction.

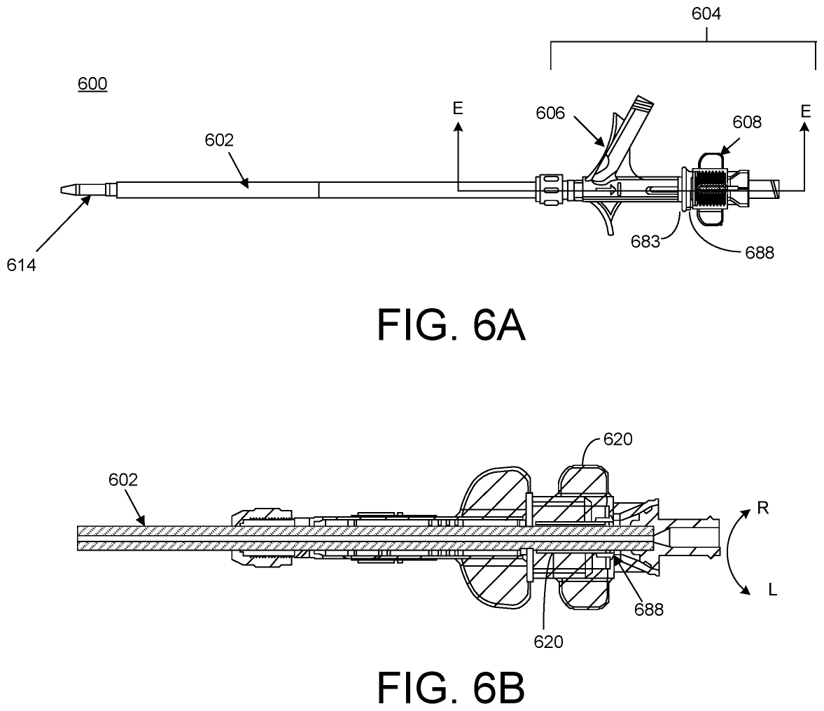

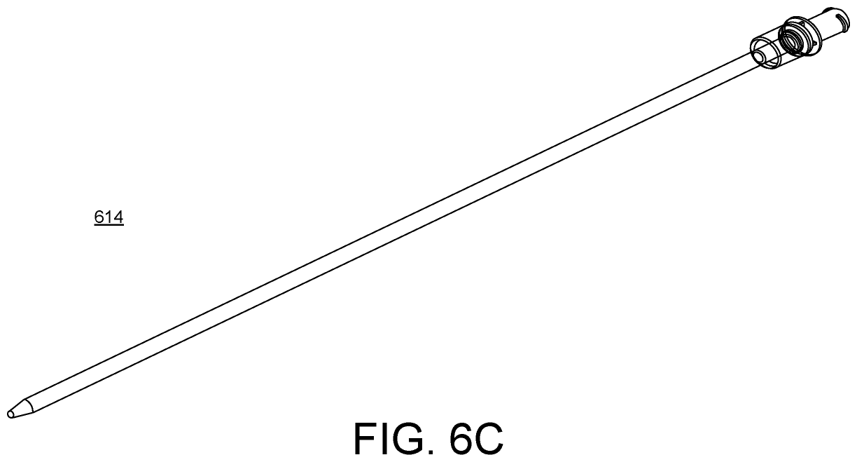

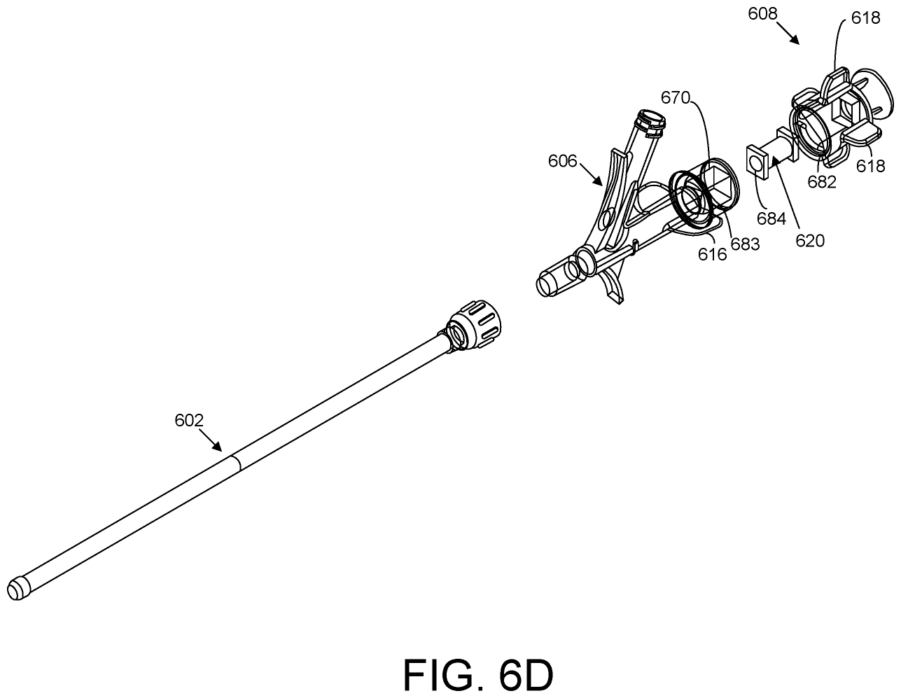

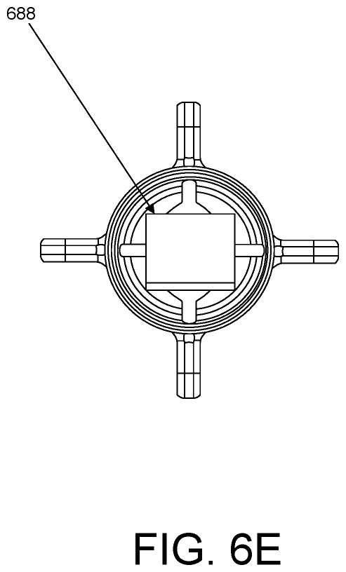

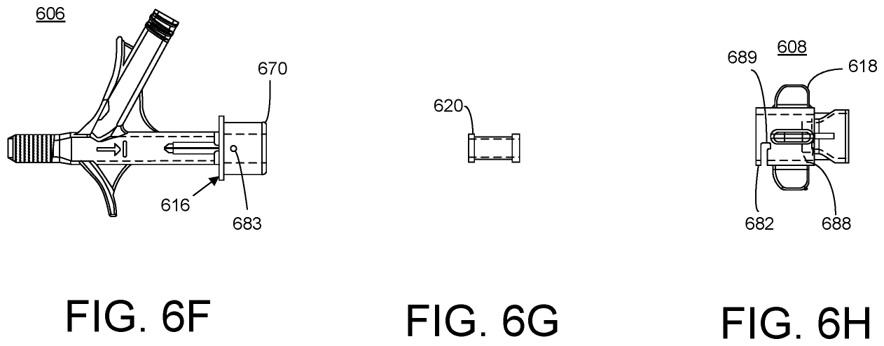

FIGS. 6A through 6H illustrate an example of a sheath device 600 according to another aspect. The sheath device 600 may be an example of any of the sheath devices discussed herein and may include any of the details discussed with reference to the other figures. The sheath device 600 is configured to facilitate the removal of stones, stone fragments, or other debris from the human body (e.g., the kidney, urinary tract, etc.). In some examples, the sheath device 600 may be configured to be used with a dilator 614 to place a portion of the sheath device 600 into the human body (e.g., the kidney, urinary tract). The dilator 614 may be similar to the dilator 614 of FIGS. 2A through 2J and may include any of the details discussed with reference to those figures.

FIG. 6A illustrates a side perspective of the sheath device 600 with the dilator 614 disposed within the lumen of the sheath device 600. FIG. 6B illustrates a cross-sectional view of the sheath device 600 taken along a line E-E. FIG. 6C illustrates an example of the dilator 614 that is used to insert a portion of the sheath device 600 into the body of a patient. The sheath device 600 includes a sheath shaft 602 and a valve assembly 604. The valve assembly 604 includes a connector 606, an elastic member 620 (e.g., a valve), and a cap member 608. FIG. 6D illustrates an example of the sheath shaft 602, the connector 606, the elastic member 620, and the cap member 608. FIG. 6E illustrates a perspective of the cap member 608 having a cavity 688 (e.g., a box cavity) (e.g., a square box). FIG. 6F illustrates an example of the connector 606. FIG. 6G illustrates an example of the elastic member 620 configured to operate as a valve. FIG. 6H illustrates an example of the cap member 608.

In some examples, the elastic member 620 is compressed, or the lumen 684 is narrowed by twisting the elastic member 620 about its longitudinal axis. In some examples, the elastic member 620 includes a portion that has a non-circular shape. In some examples, the elastic member 620 may have a polygon shape such as a square at each end portion of the elastic member 620. For example, a first end portion of the elastic member 620 may be disposed (e.g., held) in a cavity 688 (e.g., a cavity) in the cap member 608 and a second end portion of the elastic member 620 is disposed (e.g., held) in a cavity 670 (e.g., a square cavity) in the connector 606. In some examples, the cavity 688 has a shape that corresponds to a shape of a first end portion of the elastic member 620. In some examples, the cavity 670 has a shape that corresponds to a shape of a second end portion of the elastic member 620.

The cap member 408 may include one or more fins 618. In some examples, the cap member 608 includes a slot 682. In some examples, the connector 606 includes a pin 683, where a portion of the pin 683 may move in the slot 682. In some examples, the connector 606 includes a ring member 616. In some examples, rotation of the cap member 608 and twist of the elastic member 620 may be limited (e.g., restricted) by the slot 682 on the cap member 608 and the pin 683 on the connector 606. In some examples, the slot 682 includes a helical slot to allow a larger revolution. In some examples, cap rotation in the direction of R may twist the elastic member 620, thereby closing the lumen 684 of the elastic member 620. In some examples, the cap member 608 includes one or more lock tabs 689. The lock tab(s) 689 may be spaced along the length of the slot 682 to hold the rotation position of the cap member 608. Rotation of the cap member 608 in the direction of arrow L may untwist the elastic member 620, thereby opening the lumen 684 of the elastic member 620.

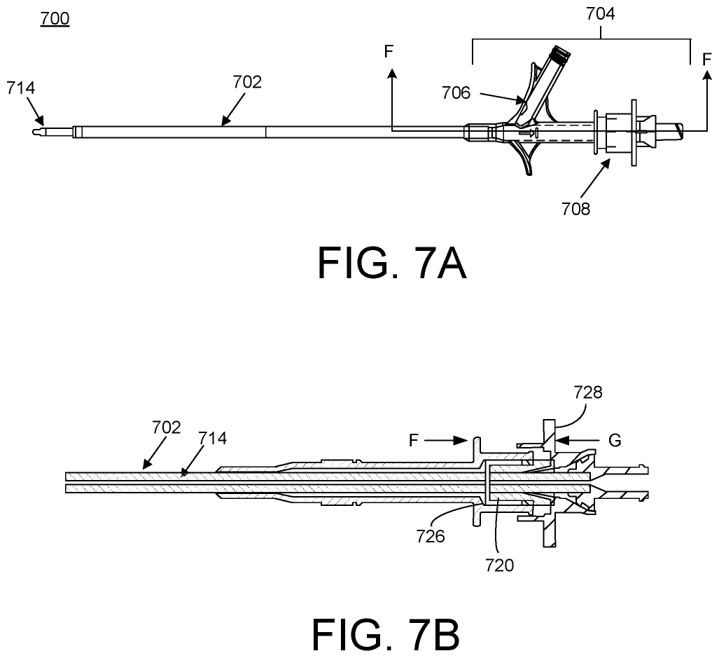

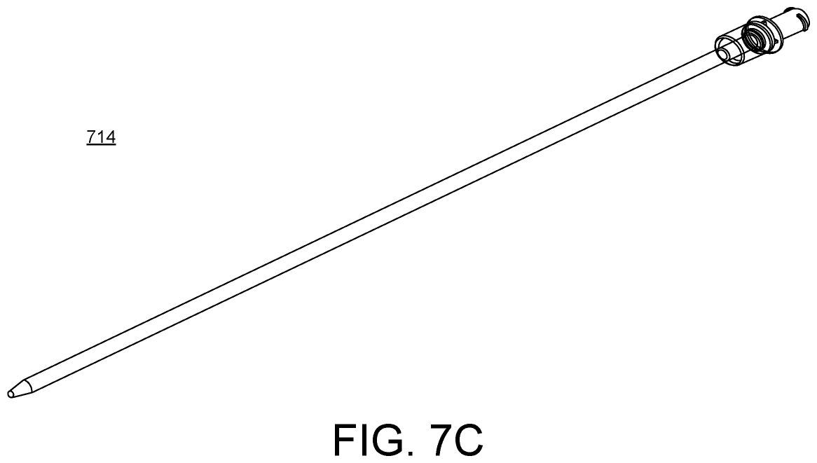

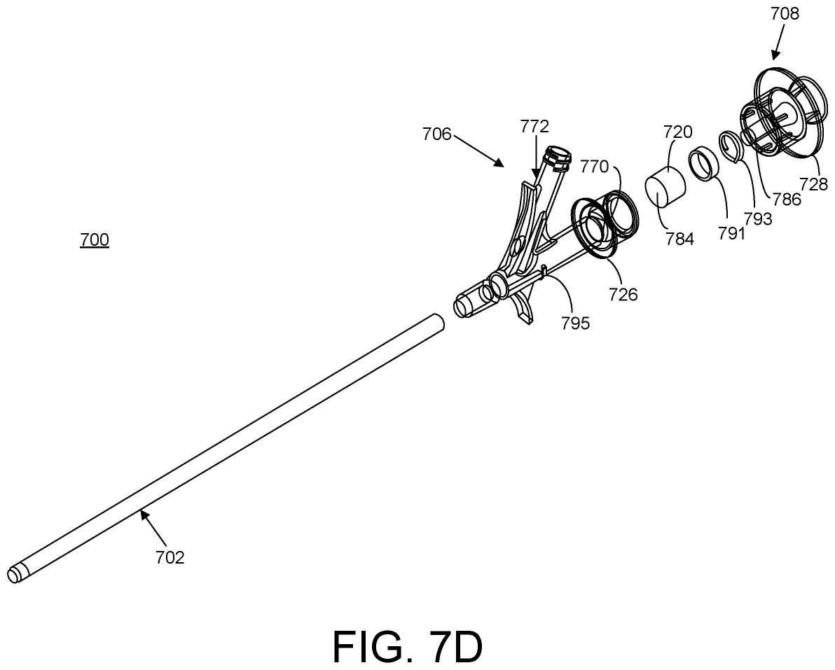

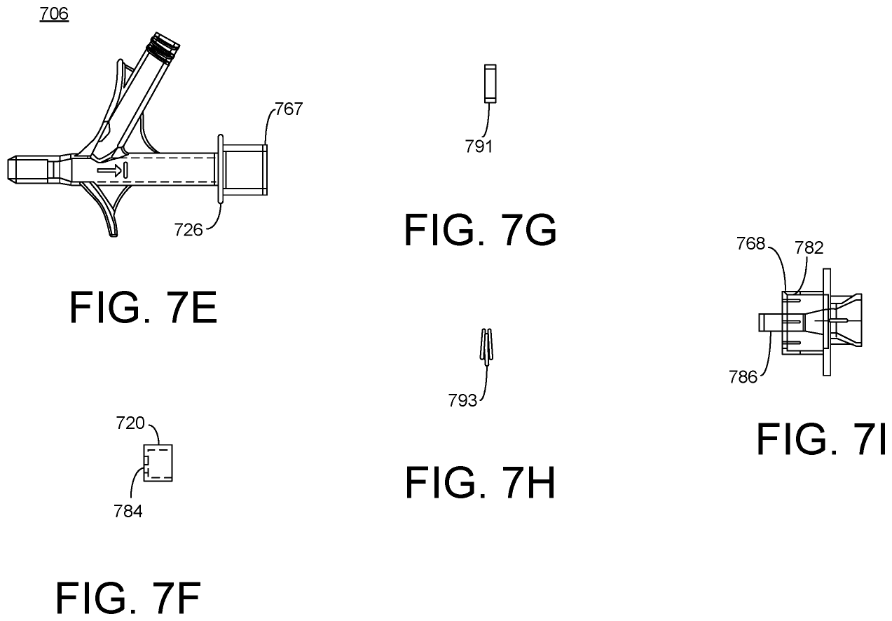

FIGS. 7A through 7I illustrate an example of a sheath device 700 according to another aspect. The sheath device 700 may be an example of any of the previous and/or subsequent sheath devices and may include any of the features discussed herein with respect to those figures. The sheath device 700 is configured to facilitate the removal of stones, stone fragments, or other debris from the human body (e.g., the kidney, urinary tract, etc.). In some examples, the sheath device 700 may be configured to be used with a dilator 714 to place a portion of the sheath device 700 into the human body (e.g., the kidney, urinary tract). The dilator 714 may be similar to the dilator 614 of FIGS. 2A through 2J and may include any of the details discussed with reference to those figures.

FIG. 7A illustrates a side perspective of the sheath device 700 with the dilator 714 disposed within the lumen of the sheath device 700. FIG. 7B illustrates a cross-sectional view of the sheath device 700 taken along a line F-F. FIG. 7C illustrates an example of the dilator 714 that is used to insert a portion of the sheath device 700 into the body of a patient. The sheath device 700 includes a sheath shaft 702 and a valve assembly 704. The valve assembly 704 includes a connector 706, an elastic member 720 (e.g., a valve), a retainer 791, a spring member 793, and a cap member 708. FIG. 7D illustrates an example of the sheath shaft 702, the connector 706, the elastic member 720, the retainer 791, the spring member 793, and the cap member 708. FIG. 7E illustrates an example of the connector 706. FIG. 7F illustrates an example of the elastic member 720. FIG. 7G illustrates an example of the retainer 791. FIG. 7H illustrates an example of the spring member 793. FIG. 7I illustrates an example of the cap member 708.

The connector 706 may include a snap ramp 767, and the cap member 708 may include a snap hook 768. In some examples, the connector 706 and the cap member 708 may be coupled together (e.g., snapped together) by the snap ramp 767 on the connector 706 and the snap hook 768 on the cap member 708.

The elastic member 720 may be disposed in a cavity 770 at a proximal end portion of the connector 706. In some examples, the elastic member 720 defines one or more slits 784 through the body of the elastic member 720. The cap member 708 includes a plunger 786. The plunger 786 may have a length that is longer than the plungers of the previous examples. The plunger 786 defines a lumen. In some examples, the cap member 708 includes one or more relief slots 782. When the cap member 708 is depressed, the plunger 786 moves in the direction of arrow G, causing the plunger 786 to extend through the slit 784 to widen the valve lumen to allow the shaft of elongated device to pass through the lumen of the plunger 786 and the widened slit 784. When the depressed cap member 708 is released by the hand, the spring member 793, disposed between the retainer 791 and the cap member 708, causes the cap member 708 to return to its initial position. In some examples, the retainer 791 includes a through hole and is used to support the spring member 793 and to disengage the elastic member 720 from the plunger 786 to allow the elastic member 720 to lock onto the shaft of an elongated device. To unlock the elastic member 720, the cap member 708 is depressed and held to widen the slit 784 and/or open the normally closed valve to allow removal of the shaft.

The valve may be opened with a single hand by squeezing a ring member 726 on the connector 706 and a ring member 728 on the cap member 708 or by squeezing the grips and the ring member 728. The valve is closed by the aid of the spring member 793. In some examples, the connector 706 includes an indicator marking 795 to indicate a scope limit withdrawal. The indicator marking 795 may include an arrow marking. The arrow marking may include a line and an arrow that points to the line. The indicator marking 795 may be disposed on the connector 706 (e.g., the proximal fitting). In some examples, the indicator marking 795 is distal to the arm portion 772. In some examples, the indicator marking 795 is aligned with the arm portion 772. In some examples, the indicator marking 795 is proximal to the arm portion 772. The indicator marking can be molded or stamped onto the connector 706. Also, any of the embodiments discussed with reference to the other figures may include the indicator marking 795. The scope shaft tip may be withdrawn to the indicator marking 795 to allow suctioned stones via the sheath shaft 702 to pass into and through an arm portion 772 without removing the scope shaft tip from the connector 706, which may allow for quick reinsertion of the scope shaft for the next stone fragments.

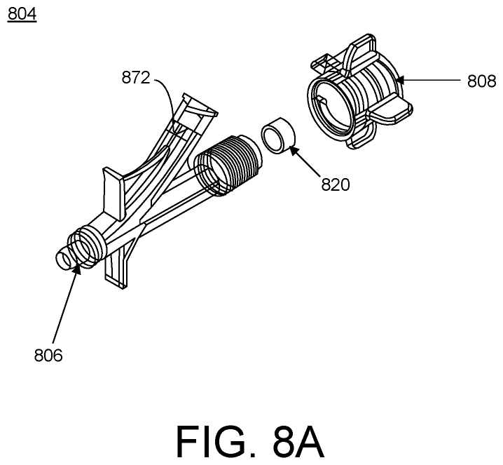

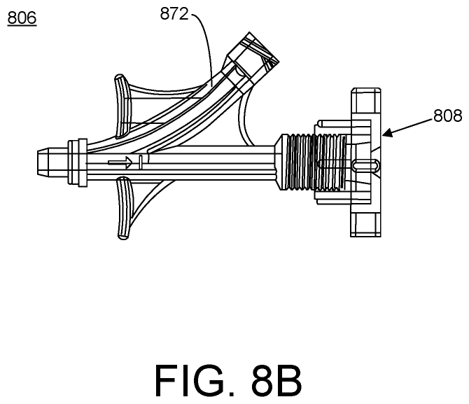

FIGS. 8A and 8B illustrate an example of a valve assembly 804 for use within any of the sheath devices discussed herein. The valve assembly 804 may be an example of any of the previous and/or subsequent sheath devices and may include any of the features discussed herein with respect to those figures. The valve assembly 804 includes a connector 806, an elastic member 820, and a cap member 808. FIG. 8A illustrates an unassembled perspective of the connector 806, the elastic member 820, and the cap member 808. FIG. 8B illustrates an assembled perspective of the connector 806, the elastic member 820, and the cap member 808. The valve assembly 804 may be similar to the valve assemblies previously discussed except that the connector 806 has an arm portion 872 that is curved.

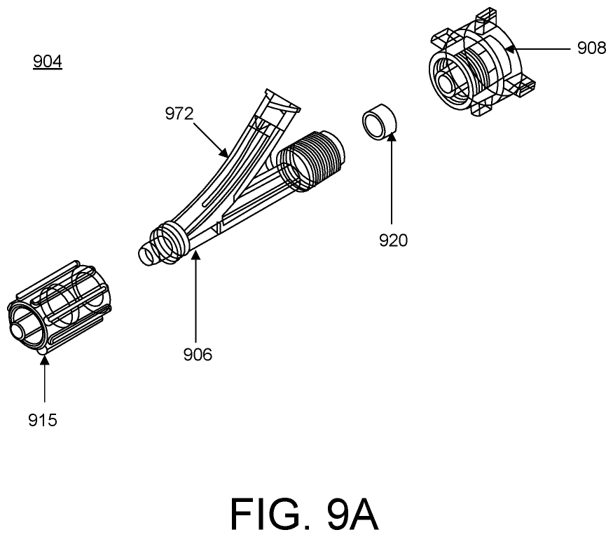

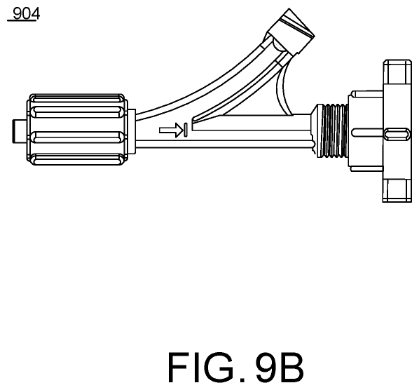

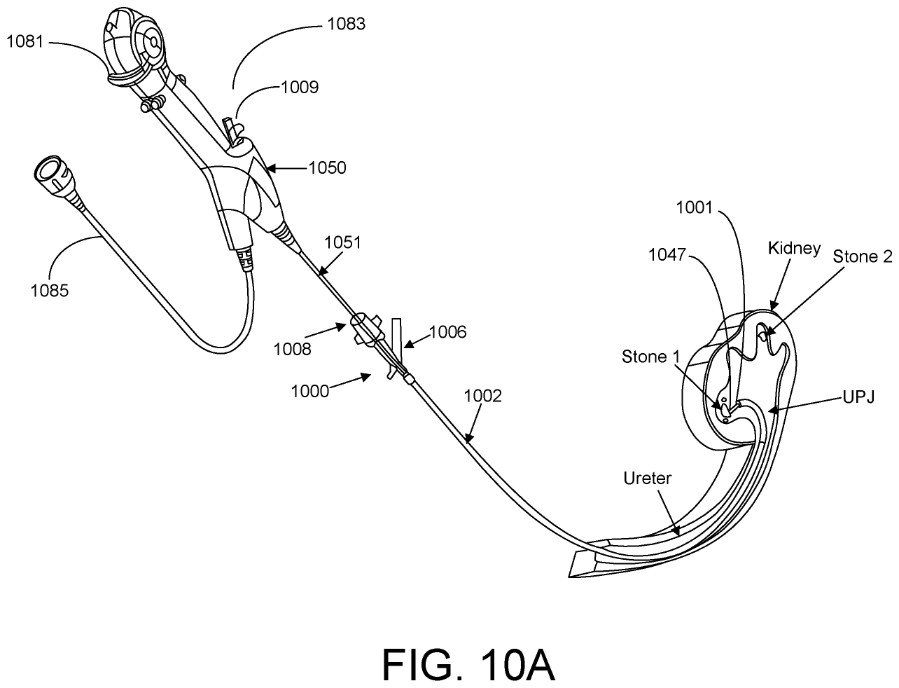

FIGS. 9A and 9B illustrate an example of a valve assembly 904 for use within any of the sheath devices discussed herein. The valve assembly 904 may be an example of any of the previous and/or subsequent sheath devices and may include any of the features discussed herein with respect to those figures. The valve assembly 904 includes a compression cap 915, a connector 906, an elastic member 920, and a cap member 908. FIG. 9A illustrates an unassembled perspective of the compression cap 915, connector 906, the elastic member 920, and the cap member 908. FIG. 9B illustrates an assembled perspective of the compression cap 915, the connector 906, the elastic member 920, and the cap member 908. The valve assembly 904 may be similar to the valve assemblies previously discussed except that the connector 906 has an arm portion 972 that is curved for ease of inserting an elongated device.

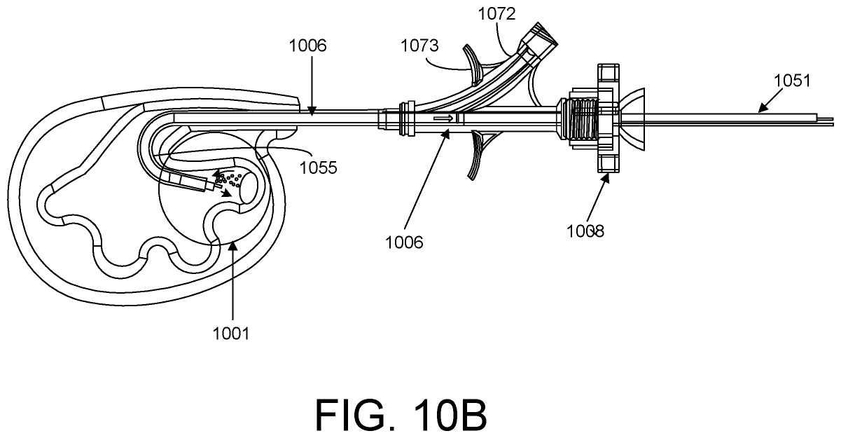

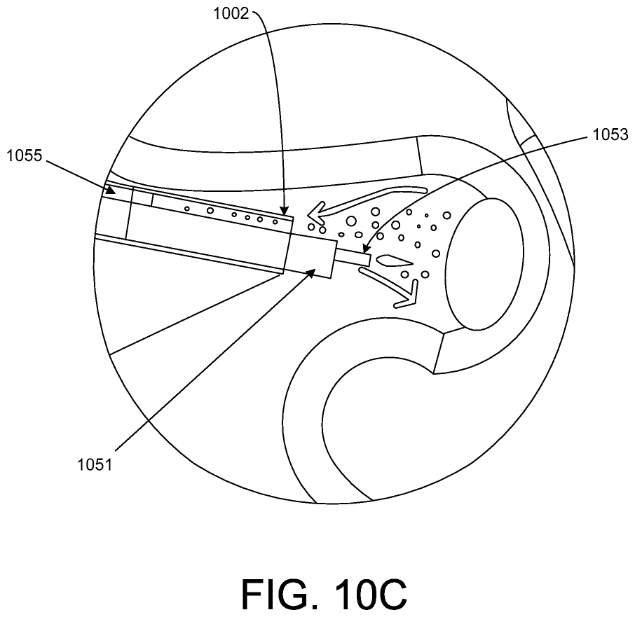

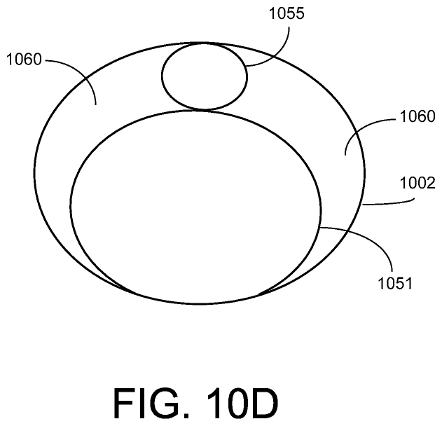

FIGS. 10A through 10D illustrate an example of a sheath device 1000 configured to be used with an insertion device 1050 according to an aspect. The insertion device 1050 may be a flexible ureteroscope. The insertion device 1050 may include a deflection knob 1081, a handle 1083, a cord 1085, and a connector 1009 (e.g., a T-shaped connector) defining a working channel and a fluid inlet. Fluid may be configured to be inserted into the body of the patient via the fluid inlet. The insertion device 1050 includes a scope shaft 1051.

The sheath device 1000 may be an example of any of the previous sheath devices and may include any of the details discussed with reference to those figures. The sheath device 1000 is configured to facilitate the removal of stones, stone fragments, or other debris from the human body (e.g., the kidney, urinary tract, etc.). The sheath device 1000 includes a sheath shaft 1002, a connector 1006, and a cap member 1008.

FIG. 10A illustrates a perspective of the sheath device 1000 with a sheath shaft 1002 disposed in the kidney. FIG. 10B illustrates a side perspective of the sheath device 1000 with the sheath shaft 1002 disposed in the kidney. FIG. 10C illustrates an enlarged view of a distal end portion 1001 of the sheath shaft 1002. FIG. 10D illustrates a cross-sectional view of the sheath shaft 1002.

Initially, using a dilator (e.g., dilator 214 or other dilators) and a guidewire 1055, the sheath shaft 1002 may be placed into the kidney or up to the ureteropelvic junction (UPJ). The dilator is then removed to allow insertion of a scope shaft 1051 into the lumen of the sheath shaft 1002. The scope shaft 1051 and the sheath shaft 1002 may be advanced in the body to be proximal to the kidney stone. The sheath shaft 1002 may then be advanced over the scope shaft 1051 to a preferred position relative to the scope tip. With the other hand that is not holding the handle 1083, the connector 1006 may be locked to the scope shaft 1051 with one hand in any of the manners discussed above.

The sheath shaft 1002 may be locked to the scope shaft 1051 such that the sheath shaft 1002 is relatively even with the scope tip 1047 or proximal to the scope tip 1047. Fluid inlet is provided through the working channel of the scope shaft 1051. A laser fiber 1053 may extend from the scope shaft 1051 to fragment or dust the renal stone as suction is applied through the lumen of the sheath shaft 1002 and govern with the finger over a hole 1073. The stone dust may be suctioned through the space (e.g., a dust channel 1060) between the inner diameter of the sheath shaft 1002 and the outer diameter of the scope shaft 1051 and then through an arm portion 1072 and into a collection system (not shown). The stone and the stone fragments may be dusted further to a smaller size to be suctioned through the space between the outer diameter of the scope shaft 1051 and the inner diameter of the sheath shaft 1002 and out through the arm portion 1072 or the stone fragment (larger than dust) may be removed as detailed below.

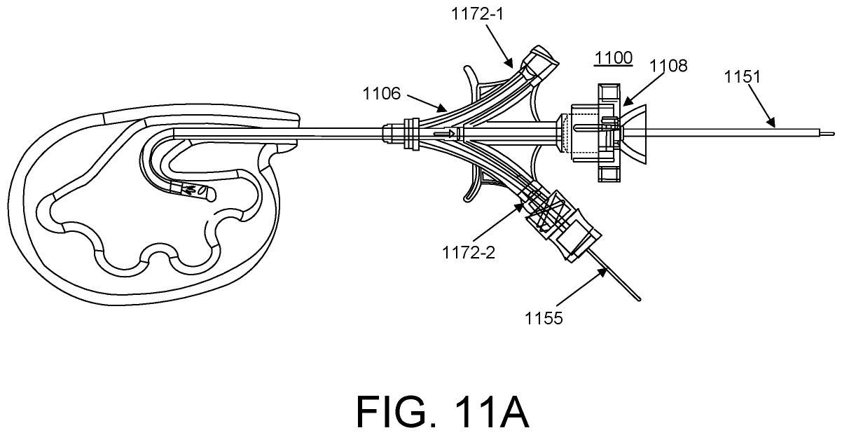

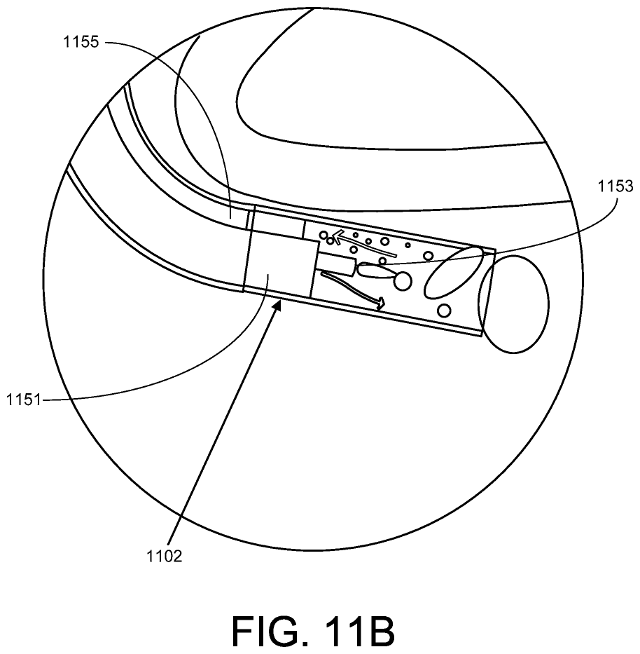

FIGS. 11A and 11B illustrate an example of a sheath device 1100 configured to be used with a scope shaft 1151 of an insertion device and an elongated device 1155 according to an aspect. The sheath device 1100 may be an example of any of the previous sheath devices and may include any of the details discussed with reference to those figures. The sheath device 1100 is configured to facilitate the removal of stones, stone fragments, or other debris from the human body (e.g., the kidney, urinary tract, etc.). The sheath device 1100 includes a sheath shaft 1102, a connector 1106, and a cap member 1108. The connector 1106 includes an arm portion 1172-1 and an arm portion 1172-2. In some examples, the arm portion 1172-1 includes a curved portion. In some examples, the arm portion 1172-2 includes a curved portion. In some examples, the elongated device 1155 is inserted into the sheath shaft 1102 via the arm portion 1172-2. In some examples, the sheath shaft 1102 may receive the elongated device 1155, the scope shaft 1151, and a laser fiber 1153. FIG. 11A illustrates a side perspective of the sheath device 1100 with the sheath shaft 1102 disposed in the kidney. FIG. 11B illustrates an enlarged view of the tip of the sheath shaft 1102.

The arm portion 1172-2 may allow the elongated device 1155 to be passed through to the distal end of the sheath shaft 1102 between the inner diameter of the sheath shaft 1102 and the outer diameter of the scope shaft 1151 (e.g., instead of or in addition to the working channel of the scope shaft 1151). The elongated device 1155 may be a laser fiber or a retrieval device, where the elongated device 1155 may be extended or retracted into and out of the sheath shaft 1102, which may free up the scope working channel to provide additional fluid volume inlet or another elongated device.

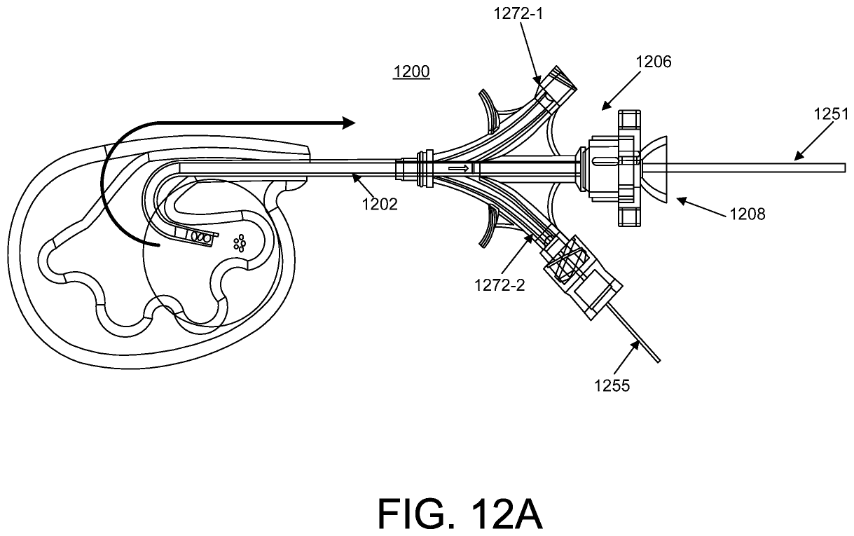

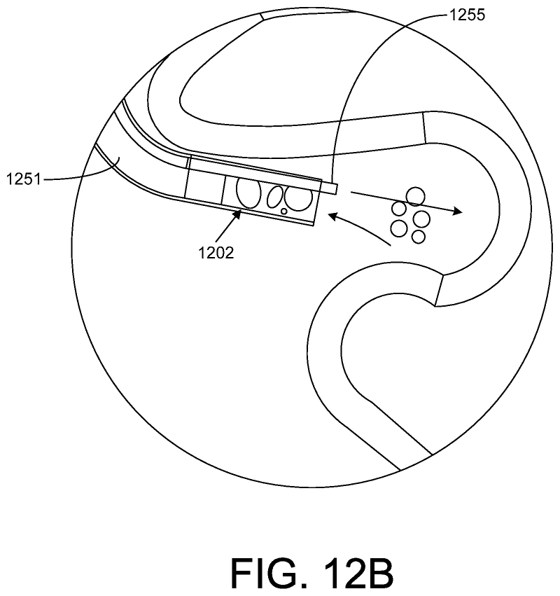

FIGS. 12A and 12B illustrate an example of a sheath device 1200 configured to be used with a scope shaft 1251 of an insertion device (e.g., ureteroscope) and an elongated device 1255 according to an aspect. The sheath device 1200 may be an example of any of the previous sheath devices and may include any of the details discussed with reference to those figures. The sheath device 1200 is configured to facilitate the removal of stones, stone fragments, or other debris from the human body (e.g., the kidney, urinary tract, etc.). The sheath device 1200 includes a sheath shaft 1202, a connector 1206, and a cap member 1208. The connector 1206 includes an arm portion 1272-1 and an arm portion 1272-2. In some examples, the arm portion 1272-1 includes a curved portion. In some examples, the arm portion 1272-2 includes a curved portion. In some examples, the elongated device 1255 is inserted into the sheath shaft 1202 via the arm portion 1272-2. In some examples, the sheath shaft 1202 may receive the elongated device 1255, and the scope shaft 1251. FIG. 12A illustrates a side perspective of the sheath device 1200 with the sheath shaft 1202 disposed in the kidney. FIG. 12B illustrates an enlarged view of the tip of the sheath shaft 1202.

In some examples, the elongated device 1255 includes a tube or a single lumen catheter. The elongated device 1255 may be round, oval, and/or smile-shape. The elongated device 1255 may be a circular shape. In some examples, the elongated device 1255 may be extended beyond the sheath tip to provide an inlet or outlet fluid lumen. In some examples, the sheath device 1200 is used to capture stone fragments having sizes too large to pass between the inner diameter of the sheath shaft 1202 and the outer diameter of the scope shaft 1251. The inlet fluid flow from the working channel of the scope shaft 1251 may be lowered or turned off this mode of use. In some examples, the cap member 1208 may be loosened (with one hand) such that it is still sealed, and the scope shaft 1251 may slide. In some examples, an elongated device 1255 is advanced beyond the distal tip of the sheath shaft 1202 and used as an inlet lumen. With suction on (and, in some examples, covering the governing hole), and the stones suctioned into the distal end of the sheath shaft 1202, withdraw the scope shaft/tip in the direction of arrow R, with the fragments following along, until it reaches the indicator marking. When the tip of the scope shaft 1251 reaches the marking, the path to the sidearm becomes opened (unobstructed by the scope tip) the fragments may be suctioned out the sidearm and into a collection system (not shown). Since the scope shaft is still in the straight Y connector lumen, the scope shaft may be quickly advanced back into the sheath lumen and to the kidney for the next fragments.

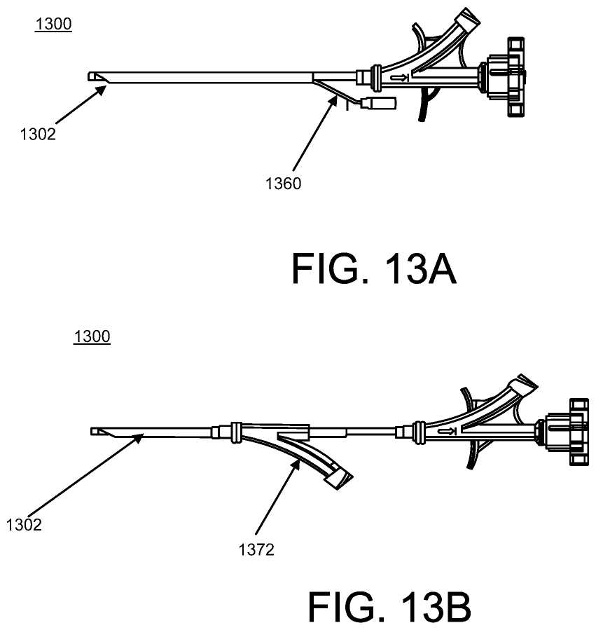

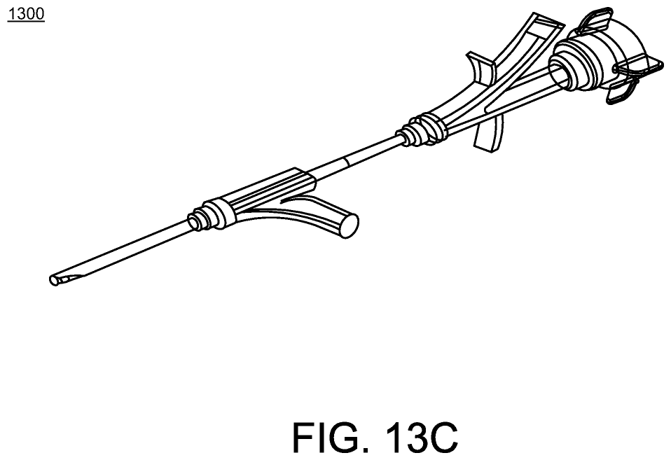

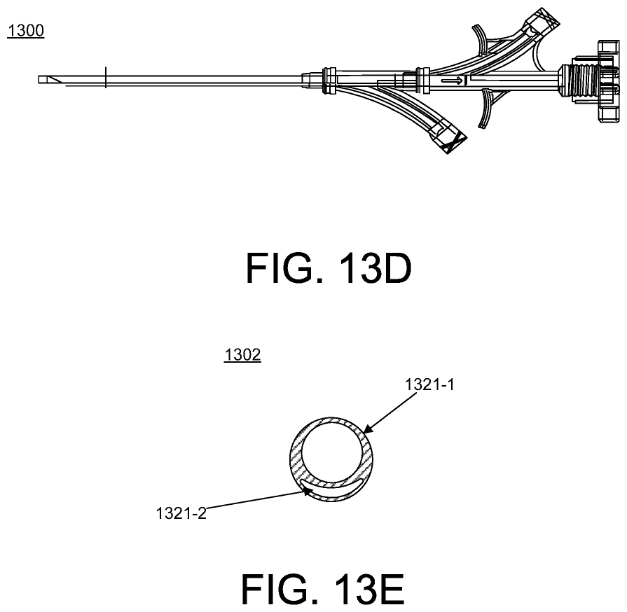

FIGS. 13A through 13E illustrate an example of a sheath device 1300 according to another aspect. The sheath device 1300 may be an example of any of the previous and/or subsequent sheath devices and may include any of the features discussed herein with respect to those figures. The sheath device 1300 is configured to facilitate the removal of stones, stone fragments, or other debris from the human body (e.g., the kidney, urinary tract, etc.). The sheath device 1300 includes a sheath shaft 1302 with two lumens, e.g., a lumen 1321-1, and a lumen 1321-2. The lumen 1321-2 may be in fluid communication with a third sidearm 1360 or a secondary connector 1372. In some examples, the lumen 1321-2 may include a sensor such as a pressure sensor or a temperature sensor. In some examples, the lumen 1321-2 may include a laser fiber and/or be used as an inlet fluid lumen or a pressure relief lumen. FIG. 13A illustrates a sheath device 1300 with a dual sheath shaft and a third sidearm 1360. FIG. 13B illustrates a sheath device 1300 with a dual sheath shaft and a secondary connector 1372. FIG. 13C illustrates two sheath devices coupled together according to an aspect. FIG. 13D illustrates two sheath devices coupled together according to another aspect. FIG. 13E illustrates a cross-sectional profile of a dual sheath shaft.

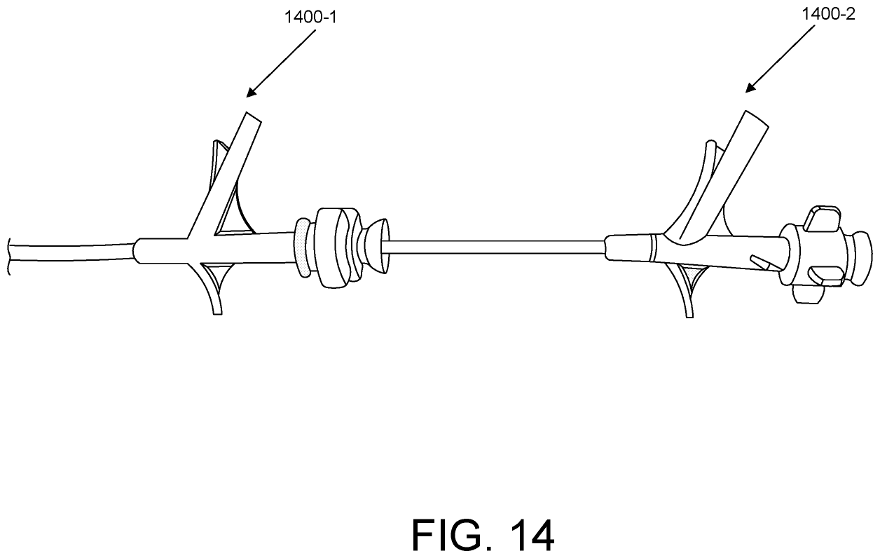

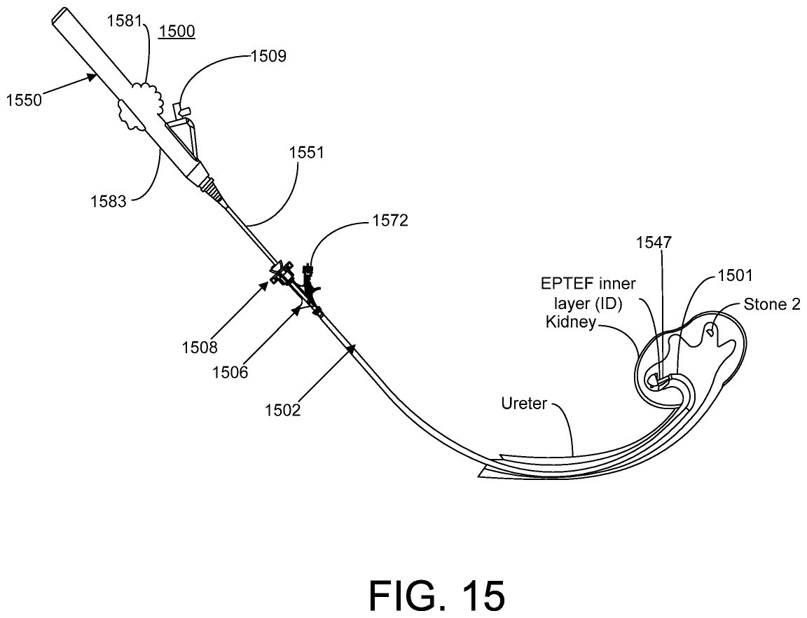

FIG. 14 illustrates an example of a system having multiple sheath devices connected in series according to an aspect. For example, the system may include a first sheath device 1400-1 that is connected to a second sheath device 1400-2. The first sheath device 1400-1 and the second sheath device 1400-2 may be examples of any of the previous and/or subsequent sheath devices and may include any of the features discussed herein with respect to those figures. In some examples, the system of FIG. 14 includes a two-channel sheath system in which the two sheaths are assembled co-axially. The first channel includes the lumen of the second sheath shaft of the second sheath device 1400-2. The second channel includes the space between the inner diameter of the first sheath shaft and the outer diameter of the second sheath shaft. The connector on the first sheath device 1400-1 may be used as the inlet port while the arm portion on the second sheath device 1400-2 may be used as the suction port. In some examples, the second sheath may be longer than the first sheath. The first sheath (inserted with a dilator) may be guided by a fluoroscope and a guidewire into the kidney and the dilator is removed. The second sheath may be inserted, assembled, and locked to the first sheath. The first sheath may inject fluid to flush stone fragments toward the second sheath while the second sheath may suction the stone particles from the distal to the proximal end and out the arm portion of the second sheath device 1400-2.

FIG. 15 illustrates a sheath device 1500 according to another aspect. The sheath device 1500 may be an example of any of the previous sheath devices and may include any of the details discussed with reference to those figures. The sheath device 1500 is configured to facilitate the removal of stones, stone fragments, or other debris from the human body (e.g., the kidney, urinary tract, etc.). The sheath device 1500 may be used with an insertion device 1550. The insertion device 1550 may include a control knob 1581, a handle 1583, a connector 1509 (e.g., a T-shaped connector) defining a working channel and a fluid inlet. The insertion device 1550 includes a scope shaft 1551.

The sheath device 1500 includes a sheath shaft 1502, a connector 1506, and a cap member 1508. The sheath shaft 1502 includes a distal end portion 1501 disposed within the kidney. The connector 1506 includes an arm portion 1572 defining an inlet port. In some examples, the sheath device 1500 is configured to be used as a fluid inlet lumen when sealed or locked to the scope shaft 1551 of the insertion device 1550. The insertion device 1550 may be a steerable device such as a flexible ureteroscope or a steerable catheter. In some examples, the insertion device 1550 includes a steerable catheter. The catheter may have two or more Bowden type pull wires to deflect its distal shaft end, in two directions via rotation of a control knob 1581 (e.g., a pull and slack control knob). In some examples, the catheter may not have an imager or lighting to allow it to have a bigger working channel and may be guided by fluoroscopic imaging.

The sheath device 1500 with the dilator may be delivered over a guidewire. The dilator is removed, and the steerable catheter is inserted into the sheath device 1500. Dust may be suctioned out through the catheter lumen while the space between the sheath shaft's inner diameter and catheter's outer diameter may provide a channel for a fluid inlet to replenish the fluid that is being suctioned out with the debris. The sheath shaft 1502 may be advanced or retracted along the catheter shaft (e.g., 1551) such that this movement directs the flush and stone towards the tip 1547 for suction removal. In some examples, the steerable catheter has a lining with an inner layer of laser resistant material to allow a laser fiber to be inserted into the main lumen to break up any clogged stone fragments blindly, safely and without damage to the catheter or injury to the patient. In some examples, the laser fiber is extended to the tip 1547 of the catheter (but not beyond) to fragment stones that are suction up to the tip 1547 but are too large to enter the catheter. In some examples, the working channel of the insertion device 1550 had a lining with an inner layer of laser resistant material.

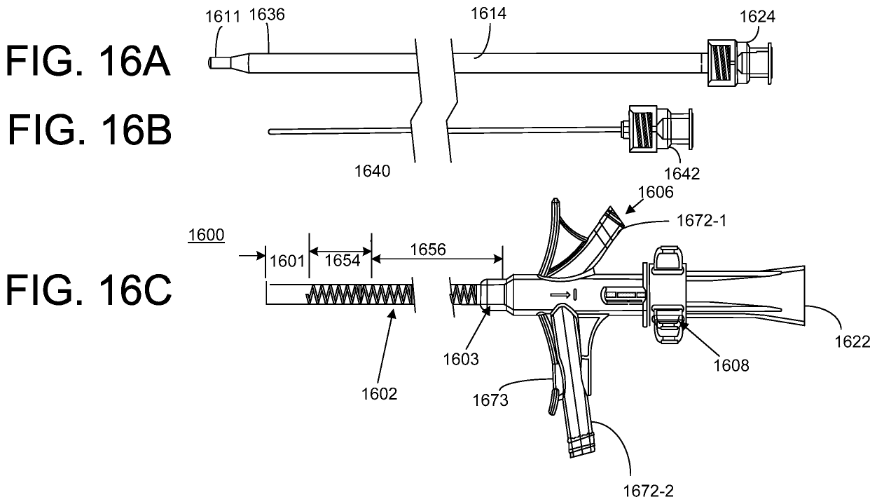

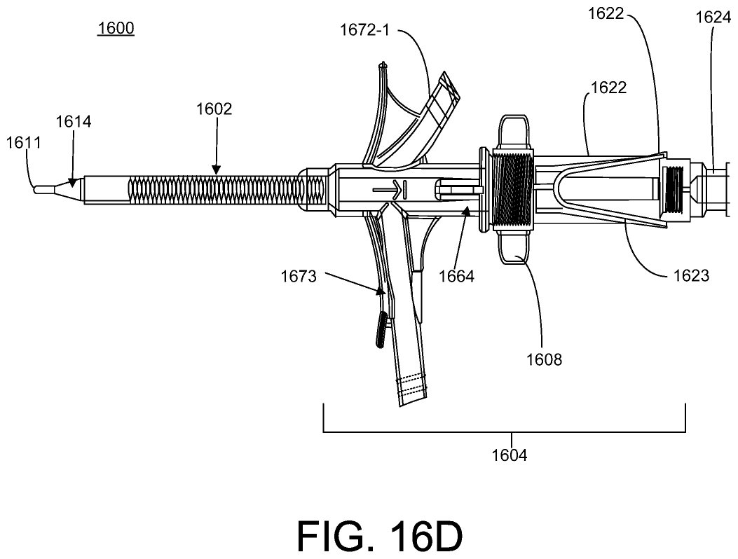

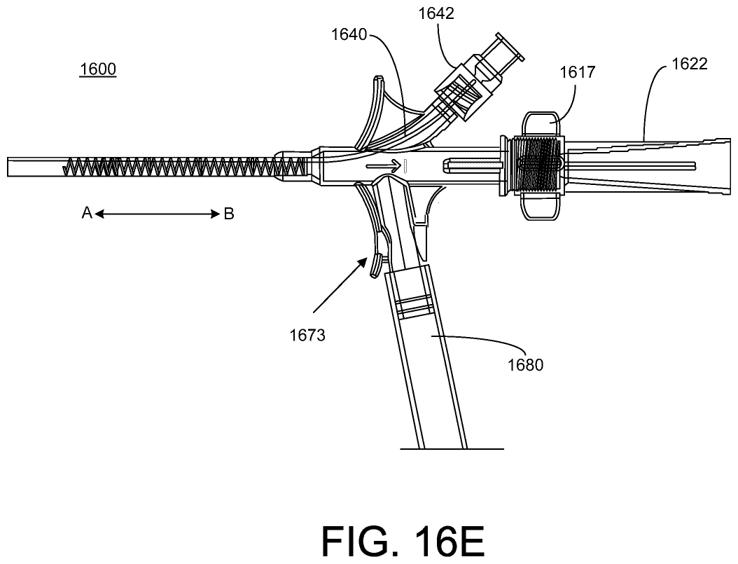

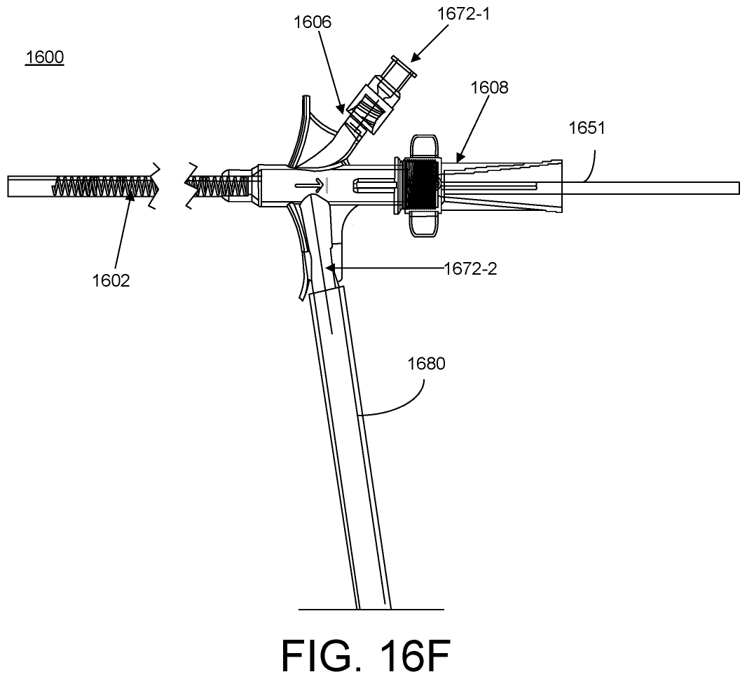

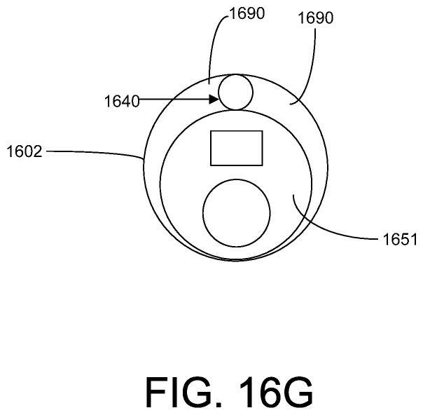

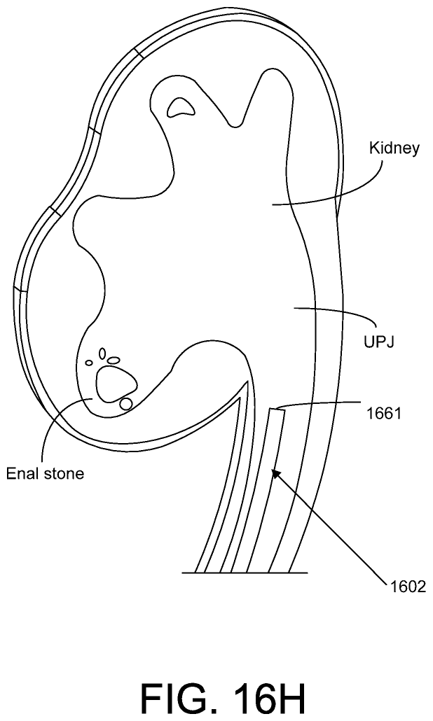

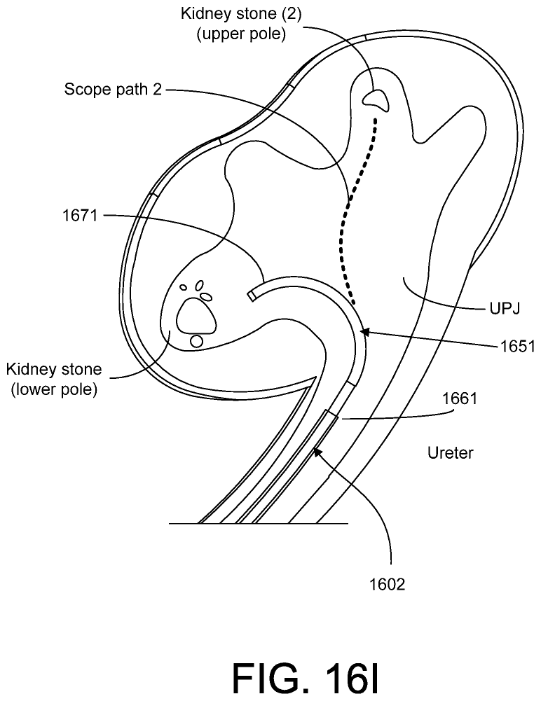

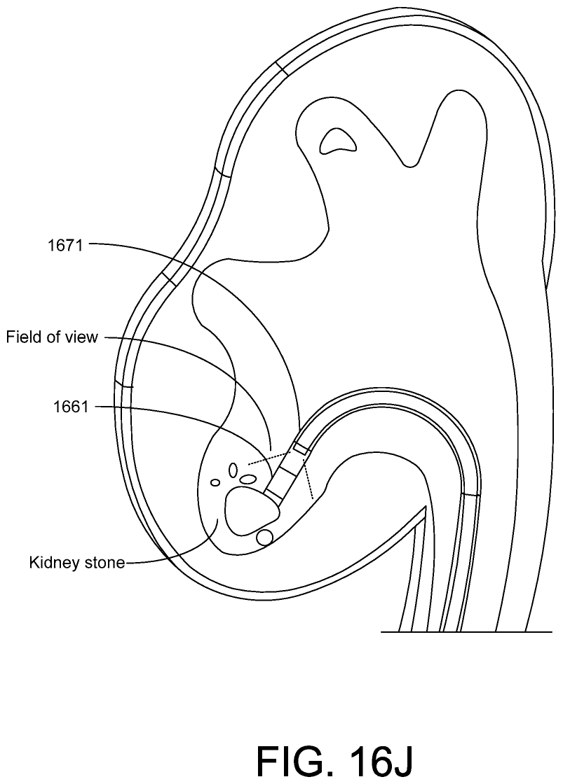

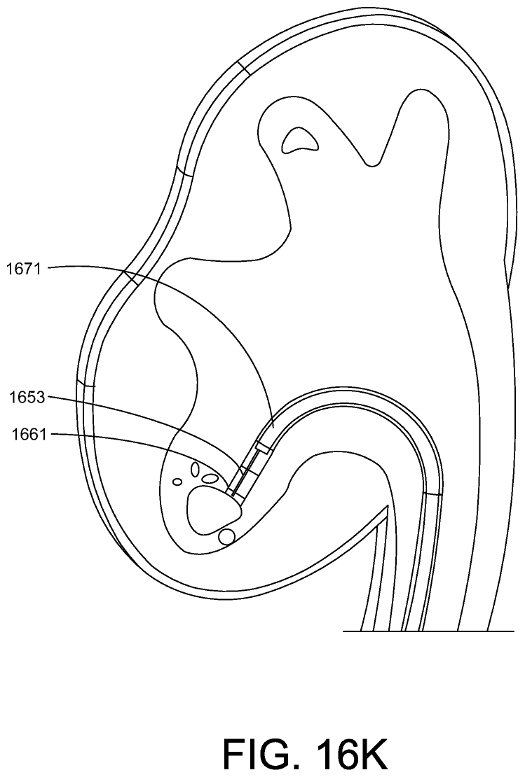

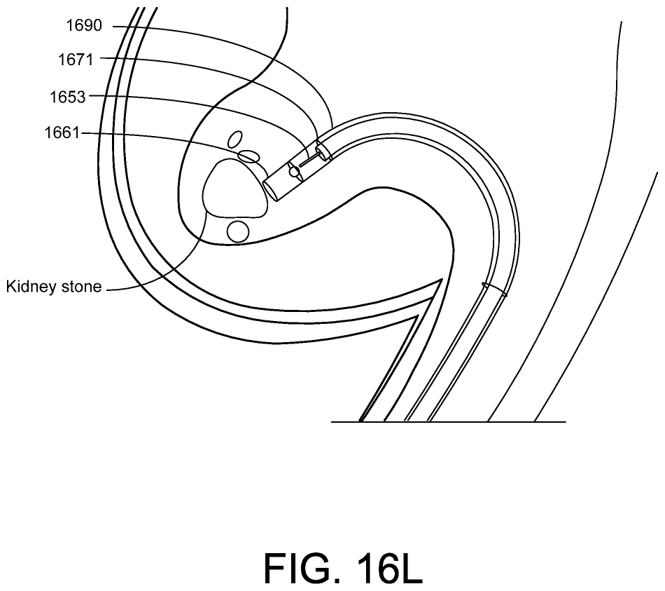

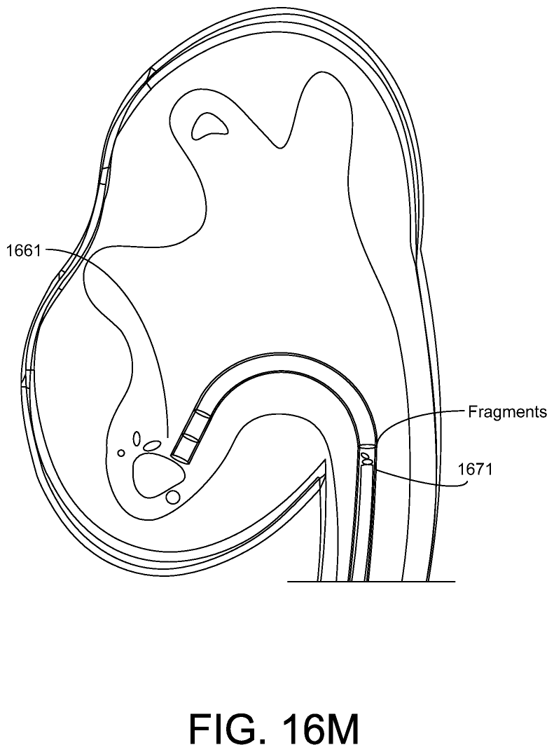

FIGS. 16A through 16M illustrate an example of a sheath device 1600 according to another aspect. The sheath device 1600 may be an example of any of the sheath devices discussed herein and may include any of the details discussed with reference to the other figures. The sheath device 1600 is configured to facilitate the removal of stones, stone fragments, or other debris from the human body (e.g., the kidney, urinary tract, etc.). In some examples, the sheath device 1600 may be configured to be used with a dilator 1614 and a biasing member 1640.

FIG. 16A illustrates the dilator 1614 according to an aspect. FIG. 16B illustrates the biasing member 1640 according to an aspect. FIG. 16C illustrates a side view of the sheath device 1600 according to an aspect. The sheath device 1600 includes a sheath shaft 1602 and a valve assembly 1604. The valve assembly 1604 includes a connector 1606 and an elastic member (not shown), and a rotation member 1608 (e.g., a knob). FIG. 16D illustrates an example of the sheath device 1600 with the dilator 1614. FIG. 16E illustrates an example of the sheath device 1600 with the biasing member 1640. FIG. 16F illustrates the sheath device 1600 according to another aspect. FIG. 16G illustrates a cross-section of the sheath shaft 1602 that includes the biasing member 1640, a scope shaft 1651, and a gap 1690. FIG. 16H depicts a sheath tip portion 1661 of the sheath shaft 1602 in the body of the patient. FIG. 16I illustrates a tip portion 1671 of the scope shaft 1651 extending from the sheath tip portion 1661. FIG. 16J illustrates a perspective of the tip portion 1671 of the scope shaft 1651 that is retracted from the sheath tip portion 1661 of the sheath shaft 1602. FIG. 16K illustrates a perspective of a laser fiber 1653 extending from the tip portion 1671, where the laser fiber 1653 extends to the tip portion 1671 to break apart larger kidney stores that may not fit within the lumen of the sheath shaft 1602. FIG. 16L illustrates a perspective of the laser fiber 1653 extending from the tip portion 1671 of the scope shaft 1651, where the end of the laser fiber 1653 is disposed within the lumen of the sheath shaft 1602. In FIG. 16L, stone fragments may be suctioned into the sheath shaft 1602 to be broken apart using the laser fiber 1653 in which the smaller fragments are removed through a working channel of the sheath shaft 1602. FIG. 16M illustrates a perspective of collecting stone fragments in the sheath shaft 1602.

The dilator 1614 may include any of the features discussed with reference to any of the dilators discussed herein. The dilator 1614 may include a lumen 1611, a tapered portion 1636, and a hub connector 1624. In some examples, the dilator 1614 includes a tapered flexible distal tip and a semi-rigid proximal end terminating with a hub connector 1624 (e.g., a medical Luer hub). The lumen 1611 extends from the distal end to the proximal end to accommodate a guidewire and fluid communication. The outer diameter of the dilator 1614 may be sized to closely fit into the inner diameter of the sheath shaft 1602 to protect the distal sheath edge. The dilator 1614 may be inserted into the sheath shaft 1602 through a funnel 1622 on the proximal end portion of the connector 1606 and may extend from the sheath tip a desired length or to the hub connector 1642. The dilator 1614 may be locked in place by rotation of a rotation member 1617 (e.g., a knob) to the lock position.

The biasing member 1640 may be an elongated member that has a diameter that is less than the dilator 1614. The biasing member 1640 may include a hub connector 1642 at a proximal end portion of the biasing member 1640. In some examples, the biasing member 1640 includes a cylindrical tube (e.g., a solid tube).

The sheath shaft 1602 may include any of the features discussed with reference to any of the sheath shafts discussed herein. The sheath shaft 1602 may include a distal end portion 1601 and a proximal end portion 1603. In some examples, the distal end portion 1601 may include an optically clear portion. In some examples, the sheath shaft 1602 may include a flexible portion 1654 and a rigid portion 1656. In some examples, the flexible portion 1654 and the rigid portion 1656 include a reinforcement member. In some examples, the reinforcement member includes a braid. In some examples, the reinforcement member includes a coil. In some examples, the sheath shaft 1602 does not include a reinforcement member. In some examples, the distal end portion 1601 includes a tapered portion. In some examples, the distal end portion 1601 includes a radiopaque marker such as a marker, ring or band. In some examples, the flexible portion 1654 and/or the rigid portion 1656 include a transparent portion. In some examples, the flexible portion 1654 includes a lower density EPTFE material. In some examples, the rigid portion 1656 includes a higher density EPTFE material. In some examples, the distal end portion 1601 includes a clear polymer. In some examples, when the distal end portion 1601 includes a clear polymer (PFA), the flexible portion 1654 and/or the rigid portion 1656 may include a EPTFE linear embedded with a tungsten coil and a polyimide variable pitch flat coil as an outer layer. In some examples, the dilator 1614 and/or the sheath shaft 1602 may be hydrophilic coated. In some examples, the dilator 1614 and/or the sheath shaft 1602 may include lubricious polymer material.

The connector 1606 may include a connector portion 1664, an arm portion 1672-1, and an arm portion 1672-2. The arm portion 1672-1 may branch (e.g., extend) from the connector portion 1664. In some examples, the arm portion 1672-1 includes a curved portion. In some examples, the arm portion 1672-2 includes a straight portion. The arm portion 1672-2 may branch (e.g., extend) from the connector portion 1664. In some examples, the connector 1606 includes a proximal end portion. In some examples, the proximal end portion includes a transparent portion. The elastic member (valve) may be opened and closed with a single hand as described with reference to the sheath devices discussed herein. The proximal fitting (e.g., the proximal portion of the connector 1606) may house one or more elastic members (valve) such as two grommets and/or wiper valves. In some examples, the proximal fitting may include one or more markings on the surface of the proximal fitting to indicate the open, seal or lock position of the valve (not shown). The hole 1673 may be located through the arm portion 1672-2. In some examples, a hole 1673 may be included on the arm portion 1672-1 and the arm portion 1672-2.

In some examples, the funnel 1622 includes a funnel slot 1623. The funnel slot 1623 enables the scope shaft to be supported by the funnel 1622. In some examples, the funnel slot 1623 may allow the scope shaft to enter the funnel 1622 along the length of the funnel slot 1623. In some examples, the funnel 1622 is configured to rotate with respect to the rotation member 1608

In some examples, biasing member 1640 may be inserted through the arm portion 1672-1 to the distal end of the sheath shaft 1602 and the hub connector 1642 is secured to the arm portion 1672-1 via a thread or slip fit. In some examples, the hub connector 1642 includes a Tuohy Borst hub. In some examples, the hub connector 1642 may be tightened onto the biasing shaft to achieve a desired length. The biasing shaft may include one or more durometers and/or materials. In some examples, the dilator shaft may be inserted into the arm portion 1672-1 and locked to the arm portion 1672-1 using the mating threads of the hub connector 1624 or other locking means. A guidewire may be in the sheath shaft 1602 to operate as a biasing member when the dilator 1614 is removed. An adaptor (e.g., a Tuohy Borst adaptor, or Urolok™ adaptor) may be used to secure the guidewire to the arm portion 1672-1. Unlocking the adaptor may allow advancement or retraction adjustment of the guidewire.

In some examples, a suction tube 1680 may be attached to the arm portion 1672-2. In some examples, the scope shaft 1651 may be inserted into the sheath shaft 1602. The tip portion 1671 of the scope shaft 1651 may be advanced beyond the sheath tip portion 1661 or may be retracted into the lumen of the sheath shaft 1602. The sheath shaft 1602 may be advanced or retracted in the direction of arrow A or B when the valve is in the seal or open position by gripping and moving the arm portion 1672-2 with the thumb and forefinger of the second hand while the first is gripping and operating the scope handle. To apply suction to the inner diameter of the sheath shaft 1602 from the suction tube 1680, the hole 1673 may be covered (or partially covered) with the forefinger and thumb of the second hand and the rest of the fingers may be wrapped about the suction tube 1680. When locking the proximal fitting to the scope shaft 1651, the hole 1673 may be positioned towards the second hand for ease of access. A laser fiber 1653 may extend from the proximal end to the distal end of the scope shaft 1651 through the working channel and the laser fiber 1653 may be positioned distal to the sheath shaft 1602 at the sheath's distal edge or within the sheath shaft 1602 to laser the kidney stones. In some examples, when dusting stones distal to the sheath shaft 1602, the edge of the sheath shaft 1602 may be positioned close to the kidney stone (e.g., close as possible) to contain and suction scattering dust to maintain a clear view.

FIG. 16G depicts a cross-sectional view of the sheath shaft 1602. The biasing member 1640 may shift (e.g., bias) the scope shaft 1651 to one side of the inner diameter of the sheath shaft 1602. The sheath shaft 1602 may include a gap 1690 (e.g., a channel) that may allow the passage of fluids and stone fragments/dust through the inner diameter of the sheath shaft 1602. The gap 1690 may also allow for renal pressure relief such as when the hole 1673 is not covered and/or for preventing over distension of the collecting system. The outer diameter of the biasing member 1640 may be sized to be less than the inner diameter of the sheath shaft 1602 minus the outer diameter of the scope shaft 1651. In some examples, the biasing member 1640 includes a mono filament (e.g. extrusion with no lumen) or a tube (e.g. extrusion with a lumen). In some examples, the biasing member 1640 may be terminated at the proximal end with the hub connector 1642.

In some examples, the biasing member 1640 includes a fixed length. In some examples, the biasing member 1640 includes a Tuohy Borst adaptor and lockable at a desirable length. In some examples, the biasing member 1640 is configured to be withdrawn or removed from the sheath shaft 1602 to free a fragment clog/bind between the scope shaft 1651 and sheath shaft 1602 to allow the scope shaft 1651 to slide once again in the sheath shaft 1602.

In some examples, the sheath shaft 1602 and dilator 1614 are inserted over a guidewire into the ureter and the UPJ. Then, the dilator 1614 may be removed. In some examples, the biasing member 1640 is then inserted into the arm portion 1672-1 and the inner diameter of the sheath shaft 1602.

In some examples, the scope shaft 1651 may be inserted past the sheath tip portion 1661 and into the kidney to be proximate to the renal stone. As shown in FIG. 16I, the scope shaft 1651 may be deflected to the lower pole stone or steered along scope path two to kidney stone (2). The user may elect to fragment the stones without the use of the sheath shaft 1602 and suction the fragments afterwards or move the stones to mid pole for easier access. The stone may be moved by using a basket or retrieval device either through the working channel of the scope shaft 1651 or through a biasing tube (e.g., a biasing member). In some examples, a basket may be used in replace of the biasing member. The stone may also be moved by suctioning the stone against the sheath edge or inside the sheath shaft 1602. The user may elect to treat the stone with the aid of the sheath shaft 1602. The sheath shaft 1602 may be advanced or slid over the deflected scope shaft 1651 to the distal end of the tip portion 1671 to be in proximity to the renal stone. In some examples, the tip portion 1671 and the sheath tip portion 1661 may be aligned at the UPJ and both the sheath shaft 1602 and scope shaft 1651 is steered by the scope shaft 1651 into the kidney in proximity to the renal stone. In some examples, the sheath tip portion 1661 is extended distal to the tip portion 1671 to be in close proximity to the stone to be fractured or dusted. Smaller stones may be suctioned towards and/or within the sheath tip portion 1661. The extended sheath tip portion 1661 may function as a funnel and an enclosure. In FIG. 16J, the dotted lines may illustrate the scope's field of view through the clear distal section of the sheath shaft 1602. In some examples, referring to FIG. 16K, the laser fiber 1653 may be extended towards the stone that is distal to the sheath tip portion 1661. In some examples, lasered stone fragments may be suctioned into the sheath shaft 1602 when the hole 1673 is covered. The inlet fluid flow is through the working channel of the scope shaft 1651.

In FIG. 16L, smaller fragments may be suctioned out of the body through the gap 1690 while larger fragments may be laser fragmented or popcorned inside the sheath shaft 1602 by retracting the laser fiber 1653 such that the stones are situated in front of the laser fiber 1653. Suction may help to prevent retropulsion of the stones/fragments, and suction may channel the stone towards the tip of the laser fiber 1653 as the sheath shaft 1602 contains the fragment within to be suctioned out through the gap 1690.

In FIG. 16M, the user may elect not to further fragment the stones within sheath shaft 1602. With the hole 1673 covered, the fragments are suctioned to the tip portion 1671 of the scope shaft 1651 as the scope shaft 1651 is withdrawn to the proximal end. When the tip portion 1671 reaches the stop line marking on the proximal fitting. The suction from the suction connection tube may cause the fragment to be suctioned into the arm portion 1672-2 and into a fluid waste canister (not shown). With the tip portion 1671 still inside the proximal fitting and supported by the funnel, the scope shaft 1651 may quickly be reinserted to the distal end of the sheath shaft 1602 for the next fragment. Removal of the scope shaft 1651 from (e.g., entirely from) the proximal fitting may cause a loss of suction pressure within the sheath shaft 1602 and may require a longer time to reinsert the scope shaft 1651. With the sheath shaft 1602 still extended distal to the tip portion 1671, the sheath shaft 1602 may be maneuvered and used after the lithotripsy procedure to suction any fragment and/or dust that are missed. It should be understood that an operator may have many opinions in which to treat renal stones with this device and may select the method of their choice. In some examples, the operator may elect to dust and suction the dust through the gap 1690. In some examples, the operator may fragment and suction withdraw the fragment upon withdrawal of the scope shaft 1651. The operation may elect to use this device to treat renal stones in the ureters or use it for ureteral access and basket the stones.

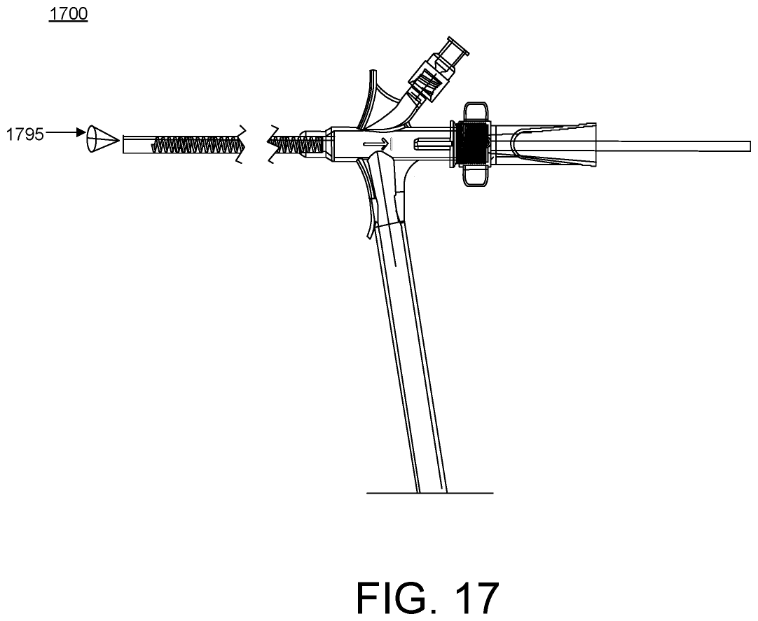

FIG. 17 illustrates an example of a sheath device 1700 according to another aspect. The sheath device 1700 may be an example of any of the sheath devices discussed herein and may include any of the details discussed with reference to the other figures. The sheath device 1700 is configured to facilitate the removal of stones, stone fragments, or other debris from the human body (e.g., the kidney, urinary tract, etc.). In some examples, the sheath device 1700 includes a basket 1795 in replace of a biasing tube.

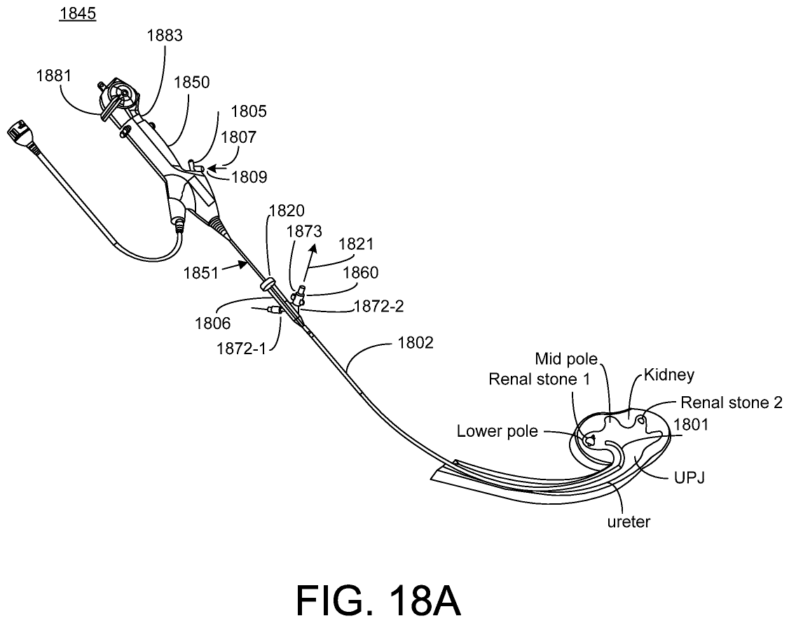

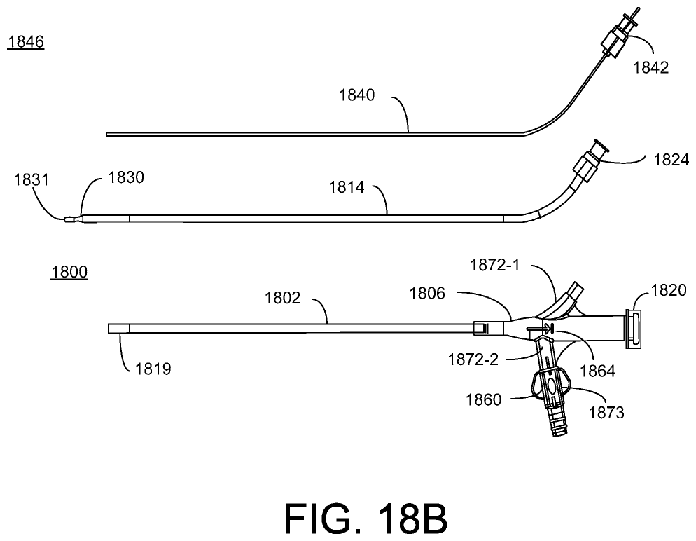

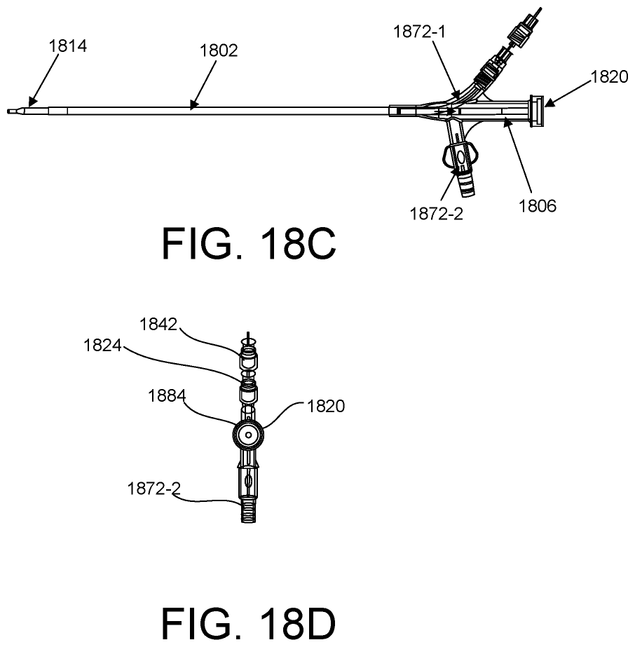

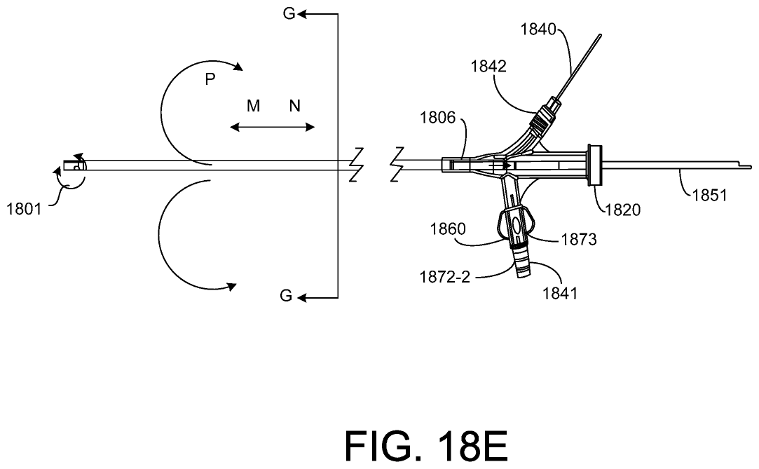

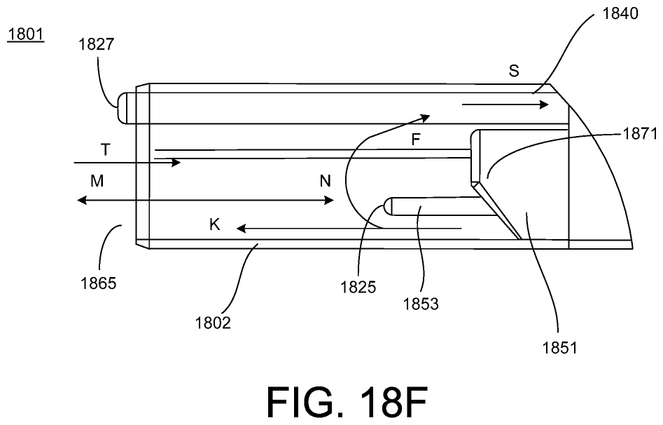

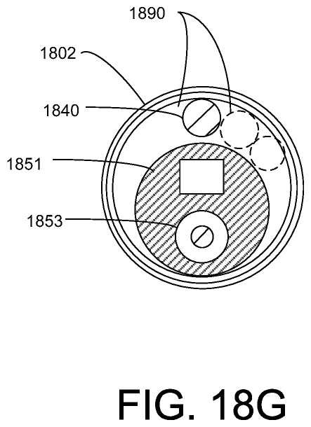

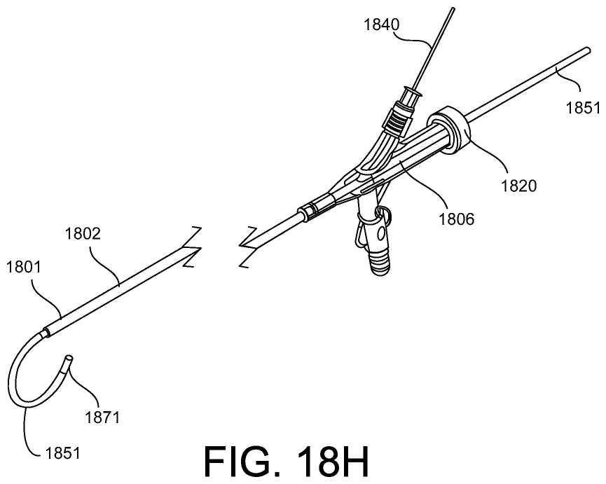

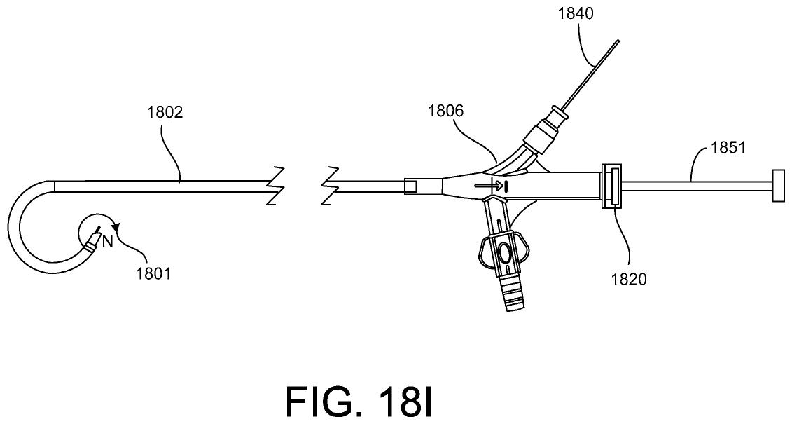

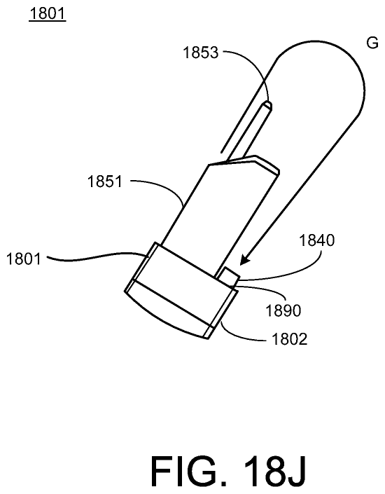

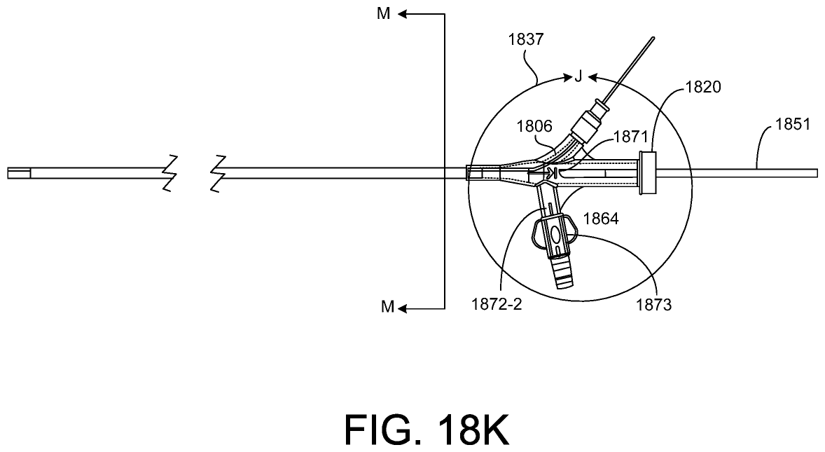

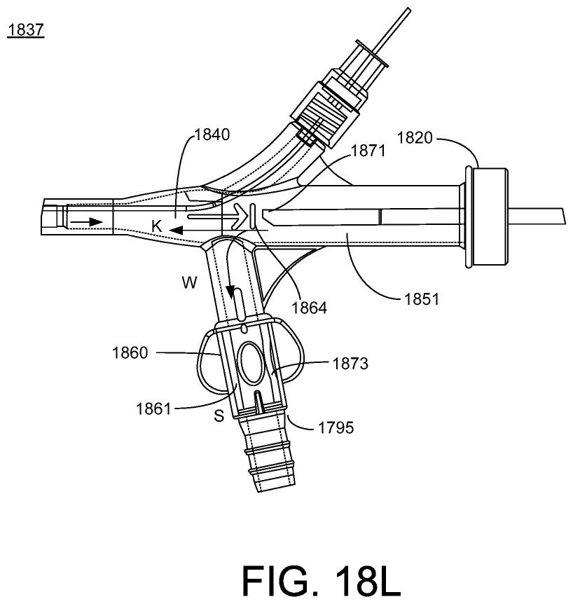

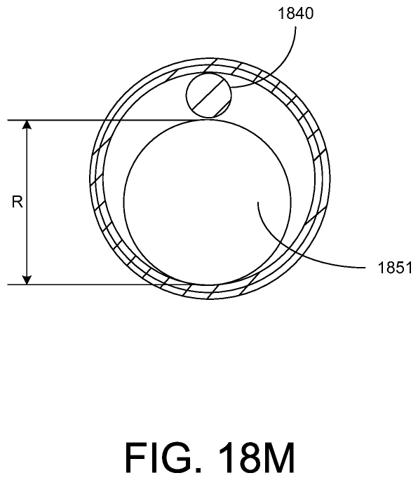

FIGS. 18A through 18M illustrate an example of a medical device 1845 having a sheath device 1800 configured to facilitate the removal of stones, stone fragments, or other debris from the human body (e.g., the kidney, urinary tract, etc.). The medical device 1845 may include an insertion device 1850, a biasing member 1840, a dilator 1814, and a sheath device 1800. The sheath device 1800 may be an example of any of the sheath devices discussed herein and may include any of the details discussed with reference to the other figures. The biasing member 1840 may be an example of any of the biasing members discussed herein and may include any of the details discussed with reference to the other figures. The dilator 1814 may be an example of any of the dilators discussed herein and may include any of the details discussed with reference to the other figures. In some examples, the sheath device 1800, the biasing member 1840, and the dilator 1814 are included as components of a sheath assembly 1846 as shown in FIG. 18B.

FIG. 18A illustrates an example of the medical device 1845 with a sheath shaft 1802 disposed in a kidney and a scope shaft 1851 disposed within the lumen of the sheath shaft 1802. FIG. 18B illustrates an exploded view of the sheath assembly 1846. The sheath assembly 1846 includes a biasing member 1840, a dilator 1814, and a sheath device 1800. FIG. 18C illustrates a packaged configuration of the sheath assembly 1846 according to an aspect. FIG. 18D illustrates a packaged configuration of the sheath assembly 1846 according to another aspect. In some examples, before the medical device 1845 is inserted into the body of the patient, the biasing member 1840 is removed and the dilator 1814 is attached to the arm portion 1872-1. Then, the sheath shaft 1802 and the dilator 1814 is inserted into the body of the patient. Once the sheath shaft 1802 is inserted into the target area, the dilator 1814 is exchanged for the biasing member 1840.

FIG. 18E illustrates a perspective of the scope shaft 1851 being inserted into the sheath shaft 1802 when the sheath shaft 1802 is disposed in the body of the patient. FIG. 18F illustrates an enlarged portion of a distal end portion 1801 of the sheath shaft 1802. FIG. 18G illustrates a cross-sectional view of the sheath shaft 1802 taken along line G-G. FIG. 18H illustrates a perspective of the sheath device 1800 in which a tip portion 1871 of a scope shaft 1851 extends away from the distal end portion 1801 of the sheath shaft 1802. FIG. 18I illustrates a perspective of the sheath device 1800 in which a tip portion 1871 of the scope shaft 1851 is proximate to the distal end portion 1801 of the sheath shaft 1802. FIG. 18J illustrates an enlarged view of the distal end portion 1801 of the sheath shaft 1802. FIG. 18K illustrates a perspective of the sheath device 1800 when the tip portion 1871 of the scope shaft 1851 is withdrawn to a withdrawal stop marker 1864 on a connector 1806 of the sheath device 1800. FIG. 18L illustrates an enlarged view of a portion 1837 of the sheath device 1800. FIG. 18M illustrates a cross-sectional view of the sheath shaft 1802 taken along line M-M.

The insertion device 1850 may be a flexible ureteroscope. Referring to FIG. 18A, the insertion device 1850 may include a scope shaft 1851, a deflection knob 1881, and a handle 1883. In some examples, the insertion device 1850 includes a laser fiber 1853 configured to extend out of (or retract inside of) the scope shaft 1851. The laser fiber 1853 includes a tip 1825 that may extend from the scope shaft 1851. The insertion device 1850 may include a connector 1809 (e.g., a T-shaped connector) defining a working channel 1805 and a fluid inlet 1807. Fluid may be configured to be inserted into the body of the patient via the fluid inlet 1807.

Referring to FIG. 18B, the biasing member 1840 may be an elongated member that has a diameter that is less than the dilator 1814. The biasing member 1840 may include a hub connector 1842 at a proximal end portion of the biasing member 1840. In some examples, the biasing member 1840 includes a cylindrical tube (e.g., a solid tube). In some examples, the biasing member 1840 does not define a lumen. In some examples, the hub connector 1842 includes a Tuohy Borst hub. In some examples, the hub connector 1842 is releasable to allow the working length of the biasing member 1840 to be adjustable such that the distal end portion of the biasing member 1840 can extend distal or proximal to the distal end portion 1801 of the sheath shaft 1802.

The biasing member 1840 may include one or more durometers and/or materials. The biasing member 1840 may include a single polymer material or more than one polymer material. The biasing member 1840 may include a dual durometer extrusion with a distal end portion being more flexible than a proximal end portion. In some examples, the biasing member 1840 may include a solid or a tubular extrusion. The biasing member may be a guidewire, a tube, or a retrieval device. The biasing member 1840 may shift (e.g., bias) the scope shaft 1851 to one side of the inner diameter of the sheath shaft 1802 (as shown in FIG. 18G). Referring to FIG. 18G, the sheath shaft 1802 may include a gap 1890 (e.g., a channel) that may allow the passage of fluids and stone fragments/dust through the inner diameter of the sheath shaft 1802. The gap 1890 may also allow for renal pressure relief such as when a hole 1873 is not covered and/or for preventing over distension of the collecting system.

Referring to FIG. 18B, the dilator 1814 may be an elongated member. The dilator 1814 may define a lumen 1831. The lumen 1831 may be a through-lumen that extends between and through the distal end of the dilator 1814 and the proximal end of the dilator 1814. In some examples, the dilator 1814 includes a tapered distal end portion 1830. In some examples, the dilator 1814 is a tubular member. In some examples, the dilator 1814 includes a hub connector 1824 at the proximal end portion of the dilator 1814. In some examples, the hub connector 1824 includes a cylindrical portion that has a size that is larger than the outer diameter of the sheath shaft 1802. In some examples, the hub connector 1824 includes a Luer hub.

Referring to FIG. 18B, the sheath device 1800 includes a connector 1806, a sheath shaft 1802, and an elastic member 1820. In some examples, the connector 1806 is a proximal fitting with two side arms. The connector 1806 defines a lumen that extends from the distal end of the connector 1806 to the proximal end of the connector 1806. The sheath shaft 1802 is connected to the distal end portion of the connector 1806, and the elastic member 1820 is connected to the proximal end portion of the connector 1806. In some examples, the elastic member 1820 is securely disposed in a housing (e.g., a cavity) of the connector 1806 at the proximal end portion of the connector 1806. The elastic member 1820 defines a lumen 1884 configured to receive the scope shaft 1851. In some examples, the lumen 1884 has a size (e.g., diameter) that is slightly less than the size of the scope shaft 1851, where the elastic member 1820 is configured to restrict movement of the scope shaft 1851. However, upon application of sufficient force, an operator can move the scope shaft 1851 through the lumen 1884.

The elastic member 1820 is configured to operate as a seal. The elastic member 1820 may be attached to the proximal end of the connector 1806 (e.g., in some examples, attached to the connector 1806 by the manufacturer). In some examples, the elastic member 1820 is not removable. In some examples, the elastic member 1820 includes a circular rib. The circular rib may assist in preventing the elastic member 1820 from being removed. In some examples, as shown in FIG. 18C, the dilator 1814 is inserted into the arm portion 1872-1 for packaging to bypass the elastic member 1820 to prevent the lumen 1884 of the elastic member 1820 from expanding to the larger diameter of the dilator 1814.

The connector 1806 includes an arm portion 1872-1 and an arm portion 1872-2. The arm portion 1872-1 and the arm portion 1872-2 may define two separate side arms. The arm portion 1872-2 defines an outlet port 1821. The arm portion 1872-1 defines a lumen that is in fluid communication with the lumen of the connector 1806. The arm portion 1872-2 defines a lumen that is in fluid communication with the lumen of the connector 1806. In some examples, the arm portion 1872-2 defines a fluid outlet 1841. In some examples, the arm portion 1872-1 is curved. In some examples, the arm portion 1872-2 is straight. The arm portion 1872-1 may include threads 1880 (e.g., female Luer threads). The arm portion 1872-2 includes a hole 1873 (e.g., a governing hole) and a rotatable hole cover 1860 configured to close or open the hole 1873. For example, the rotatable hole cover 1860 may include a cover hole 1861 that may align with the hole 1873 or may be rotated away from the 1873, thereby closing the hole 1873.

The sheath shaft 1802 may include one or more laser-resistant materials. In some examples, a laser resistant material includes expanded polytetrafluoroethylene (EPTFE). EPTFE may be microporous and is different (e.g., significantly different) to conventional PTFE tubing. In some examples, EPTFE is air-permeable, and relatively soft and flexible. In some examples, a laser resistant material may include other polymer materials such as polytetrafluoroethylene (PTFE), tetrafluoroethylene (TFE), fluorinated ethylenepropylene (FEP), perfluoroalkoy (PFA), ethylene tetra-fluoroethylene (ETFE), and polyvinylidene fluoride (PVDF). In some examples, the sheath shaft 1802 includes a transparent portion 1819 at a distal end portion 1801 of the sheath shaft 1802.

As shown in FIG. 18A, a portion of the sheath shaft 1802 is inserted into the kidney, and the scope shaft 1851 of the insertion device 1850 is inserted into the lumen of the sheath shaft 1802 to a target site. The fluid inlet path may be the working channel 1805 of the insertion device 1850 and may be accessible through the connector 1809. A fluid inlet tube (not shown) may be connected to the connector 1809 to provide or replenish fluid to the target site. The working channel 1805 may be continuously pressurized with (cooled) irrigation (e.g., in some examples, set at about 50-100 cc per minute). The fluid outlet path may be through the inner diameter of the sheath shaft 1802 and through the arm portion 1872-2. In some examples, a negative pressure is applied at the arm portion 1872-2. In some examples, a continuous negative pressure aspiration through the arm portion 1872-2 is set to be within a range (e.g., 150-200 mm Hg pressure).

In some examples, the hole 1873 through the arm portion 1872-2 is used to control the magnitude of the negative pressure that will be applied to the inner diameter of the sheath shaft 1802. Covering the hole 1873 may increase the negative pressure, while an uncovered or partially covered hole 1873 may decrease negative pressure. The user may use her thumb of a second hand or use the rotatable hole cover 1860 to cover the hole 1873 while the first hand operates the handle 1883. In some examples, the distal end portion 1801 of the sheath shaft 1802 is passive and flexible. The distal end portion 1801 of the sheath shaft 1802 may be steered or maneuvered by a tip portion 1871 of the scope shaft 1851 by activating the deflection knob 1881 of the handle 1883. In some examples, the distal end portion 1801 of the sheath shaft 1802 may be advanced over the distal portion of the scope shaft 1851 such that the distal end portion 1801 of the sheath shaft 1802 conforms to the shape of the deflected scope shaft 1851, where the sheath tip is positioned closer to the targeted site. In some examples, the distal end portion 1801 of the sheath shaft 1802 may be retracted proximally such that the distal end portion 1801 of the sheath shaft 1802 conforms to the shape of the proximal portion of the scope shaft 1851, where the sheath tip is further away from the target site.

As indicated above, FIGS. 18C and 18D depict aspects of a packaged configuration of the sheath assembly 1846. The dilator 1814 may be used to support and/or protect the sheath shaft 1802 and/or the biasing member 1840. In some examples, the biasing member 1840 is removed and the dilator 1814 is attached to the arm portion 1872-1, and the sheath shaft 1802 and the dilator 1814 is inserted into the body of the patient. Once the sheath shaft 1802 is inserted into the target area, the dilator 1814 is exchanged for the biasing member 1840.

As shown in FIG. 18E, when the scope shaft 1851 is inserted into the distal end portion 1801 of the sheath shaft 1802, the distal end portion 1801 may be deflected in the direction of arrow P and D with the insertion device 1850 (e.g., a dual directional flexible ureteroscope). The sheath shaft 1802 may also slide over the scope shaft 1851 in the direction of arrow M or N. The scope shaft 1851 may be rotated in the lumen of the sheath shaft 1802.

Referring to FIG. 18F, when the scope shaft 1851 is position inside of the sheath shaft 1802, the inflow of fluid from the working channel is shown by arrow K and the outflow of fluid (negative pressure) in the gap 1890 is shown by arrow S. In some examples, the biasing member 1840 includes a tip portion 1827. The tip portion 1827 may extend distal or proximal to an edge 1865 of the sheath shaft 1802. When the magnitude of S becomes greater than K, then the flow direction of K may transition to the direction of arrow F, thereby resulting in a net negative pressure in the direction of arrow T. The sheath shaft 1802 may be extended from the tip portion 1871 of the scope shaft 1851 to form a mechanical advantage such as a funnel or a chamber and the laser fiber 1853 may be positioned by extending or retracting the laser fiber 1853 in the direction of arrow M or N to laser a stone.

Referring to FIG. 18G, when the biasing member 1840 and the scope shaft 1851 is inserted into the inner diameter of the sheath shaft 1802, the gap 1890 is the space between the inner diameter of the sheath shaft 1802, the outer diameter of the scope shaft 1851, and the outer diameter of the biasing member 1840. The dotted circle represents fragments being suctioned through the gap 1890. The laser fiber 1853 is disposed within a lumen of the scope shaft 1851.

FIG. 18H illustrates a perspective of the sheath device 1800 in which a tip portion 1871 of a scope shaft 1851 extends away from the distal end portion 1801 of the sheath shaft 1802. FIG. 18I illustrates a perspective of the sheath device 1800 in which a tip portion 1871 of the scope shaft 1851 is proximate to the distal end portion 1801 of the sheath shaft 1802. Referring to FIGS. 181, a rotatable hole cover 1860 is used to cover the hole 1873. In some examples, the hole 1873 may be located at the proximal side of the arm portion 1872-2 and the cover hole 1861 may not be aligned with the hole 1873. In some examples, the hole 1873 is covered allowing the (max) negative pressure in the arm portion 1872-2 to be applied to the inner diameter of the sheath shaft 1802. In some examples, rotating the rotatable hole cover 1860 to partially cover the hole 1873 may decrease the negative pressure. In some examples, rotating the cover hole 1861 to be in line with the hole 1873 may allow the least amount of negative pressure to be applied to the inner diameter of the sheath shaft 1802. The hole 1873 may also be covered with a finger to adjust the negative pressure. When the hole 1873 is not covered, the hole 1873 may be used as a natural drain or gravity drain extending from the distal end portion 1801 of the sheath shaft 1802, through the gap 1890 and through the hole 1873. In some examples, the hole 1873 is not covered with a cover but may be covered using a finger. The sheath device 1800 with or without a hole 1873 may be used with a fluid management system to manage inter-renal pressure and or temperature using a pressure and or a temperature sensor. The temperature and/or the pressure sensor may be in the form of a biasing member.

In some examples, the sheath shaft 1802 includes a transparent portion 1819 (e.g., as shown in FIG. 18B). The transparent portion 1819 is disposed on the distal end portion 1801 of the sheath shaft 1802. The transparent portion 1819 is configured to allow the scope image or camera to view inside of the sheath shaft 1802 through the transparent portion 1819. Referring to 18J, when the scope shaft 1851 is extended from the distal end portion 1801 of the sheath shaft 1802, the tip portion 1871 of the scope shaft 1851 may extend into a calyx and the sheath shaft 1802 may remain proximal at the UPJ. In some examples, when the scope shaft 1851 is withdrawn to the withdrawal stop marker 1864, the inner diameter of the sheath shaft 1802 may accommodate a stone or fragment about <R mm, as shown in FIG. 18M. If the biasing member 1840 is also removed, the inner diameter of the sheath shaft 1802 may accommodate a stone or fragment about <D mm. It should be understood that a smaller diameter scope may be used to either lower the outer diameter of the sheath shaft 1802 or to increase the gap 1890 or both.

Referring to FIGS. 18K and 18L, the tip portion 1871 of the scope shaft 1851 may be withdrawn to the withdrawal stop marker 1864. The inflow of fluid from the working channel is shown by arrow K and the outflow of fluid (negative pressure) in the arm portion 1872-2 is shown by arrow S. When the magnitude of S become greater than K then the flow direction of K may be transitioned to the direction of arrow W and the resultant is negative pressure T. Once the operator withdraws the scope shaft 1851 and sees the suctioned stone/fragment has suctioned into the arm portion 1872-2, the tip portion 1871 of the scope shaft 1851 may be re-insert into the sheath shaft 1802 to minimize fluid loss through the inner diameter of the sheath shaft 1802.

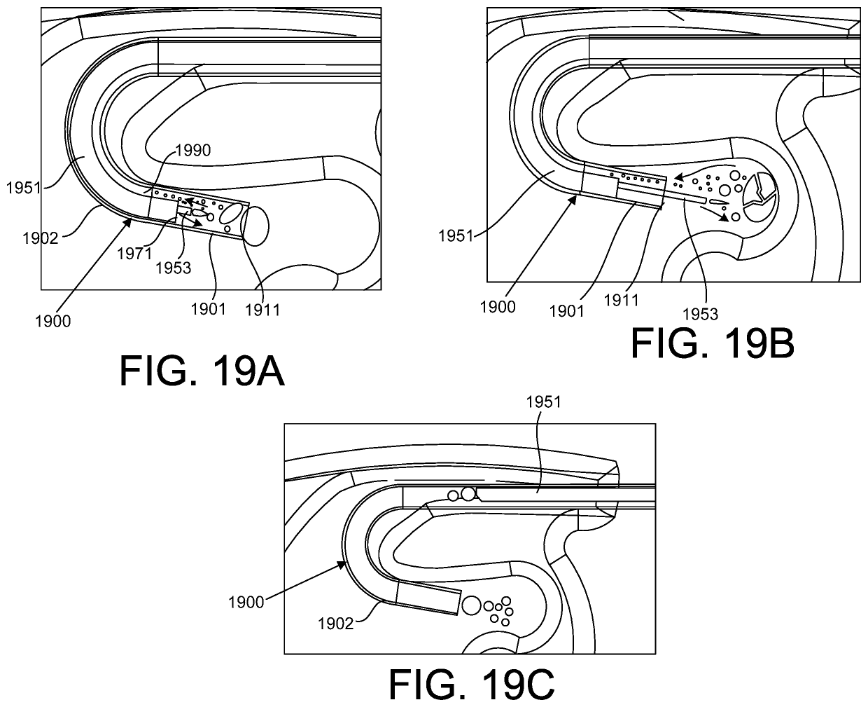

FIG. 19A illustrates a perspective of a sheath device 1900 in which stones and/or debris may be lasered inside of the inner diameter of a sheath shaft 1902 or at an edge 1911 of the sheath shaft 1902. The sheath device 1900 may be an example of any of the sheath devices discussed herein and may include any of the details discussed herein.

The dust and/or fragments may be suctioned or contained within a distal end portion 1901 of the sheath shaft 1902 and may be directly aspirated into a gap 1990 as they are lasered by a laser fiber 1953. In some examples, a portion of the laser fiber 1953 extends from an edge 1971 of a scope shaft 1951 but the portion of the laser fiber 1953 is included within a lumen defined by the distal end portion 1901. In some examples, the suction carries the stone/fragment towards the tip of the laser fiber 1953 to be lasered and may minimize retropulsion of stones. The laser energy in the form of heat may also be contained within the inner diameter of the sheath shaft 1902 and may be removed by suction. Removing fluid may decrease the renal pressure. In some examples, the extended sheath shaft 1902 may also be used as a storage area and/or to suction stones within to relocate/reposition stones to the mid or upper pole for ease of access.

FIG. 19B illustrates a perspective of a sheath device 1900 that is used to laser a stone located distal to an edge 1911 of the sheath shaft 1902. For example, a laser fiber 1953 may extend from the scope shaft 1951 to a location that is distal to the edge 1911 of the sheath shaft 1902. In some examples, the sheath shaft 1902 may be extended to create a funnel and the laser fiber 1953 may be extended to the proximity of stone for lasing. To minimize the scattering of stone fragments, the sheath shaft 1902 may be positioned closer to the stone. FIG. 19C illustrates a perspective of a sheath device 1900 in which the scope shaft 1951 is withdrawn within the sheath shaft 1902 to allow a larger fragment to be aspirated.

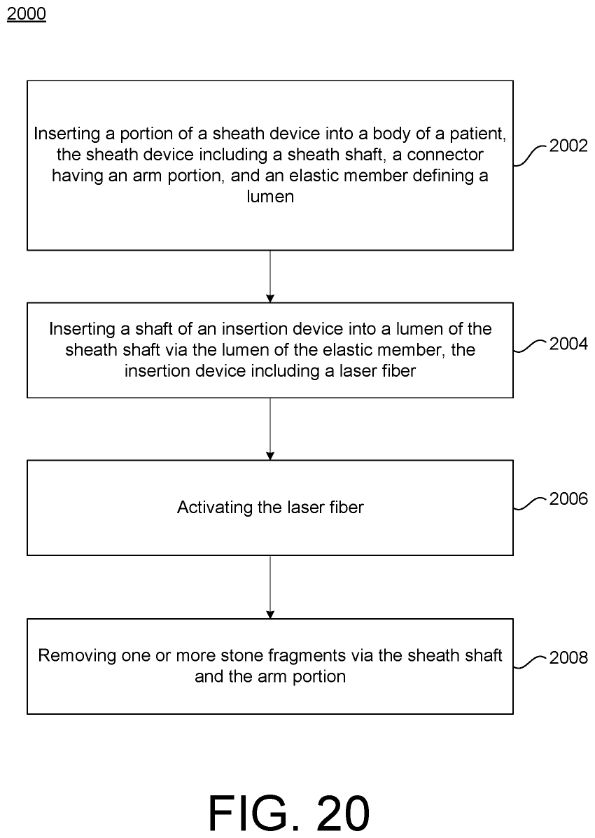

FIG. 20 illustrates a flowchart 2000 depicting example operations of inserting a sheath device into the body of the patient according to an aspect. The sheath device may be any of the sheath devices discussed herein. Although the flowchart 2000 of FIG. 20 illustrates the operations in sequential order, it will be appreciated that this is merely an example, and that additional or alternative operations may be included. Further, operations of FIG. 20 and related operations may be executed in a different order than that shown, or in a parallel or overlapping fashion. Although the flowchart 2000 is described with reference to the medical device 1845 of FIGS. 18A through 18M, the flowchart 2000 may be executed according to any of the sheath devices discussed herein.

Operation 2002 includes inserting a portion of a sheath device 1800 into the body of a patient. The sheath device 1800 includes a sheath shaft 1802, a connector 1806 having an arm portion (e.g., 1872- or 1872-2), and an elastic member 1820 defining a lumen 1884. Operation 2004 includes inserting a shaft (e.g., scope shaft 1851) of an insertion device 1850 into a lumen of the sheath shaft 1802 via the lumen 1884 of the elastic member 1820. The insertion device 1850 includes a laser fiber 1853. Operation 2006 includes activating the laser fiber 1853. Operation 2008 includes removing one or more stone fragments via the sheath shaft 1802 and the arm portion. In some examples, the removing includes aspirating one or more stone fragments via the sheath shaft 1802 and the arm portion.

In some examples, the operations include moving a tip portion of the shaft of the insertion device to a location proximal to a tip portion of the sheath shaft and suctioning a stone into a distal end portion of the sheath shaft to fragment the stone using the laser fiber from a location within the distal end portion of the sheath shaft. In some examples, the operations include extending a tip portion of the laser fiber to a location distal to a tip portion of the sheath shaft and activating the laser fiber to fragment a stone. In some examples, the arm portion is a first arm portion, and the connector includes a second arm portion, where the operations include inserting a biasing member into the sheath shaft via the second arm portion. The biasing member is configured to bias the sheath shaft away from the shaft of the insertion device within the sheath shaft.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A sheath device comprising:
a sheath shaft defining a lumen, the sheath shaft configured to be inserted into a body of a patient; and
a valve assembly coupled to a proximal end portion of the sheath shaft, the valve assembly including a connector and an elastic member, the elastic member being coupled to the connector, the connector including an arm portion that extends from a sidewall of the connector, the elastic member configured to operate as a valve, the elastic member defining a lumen.

2. The sheath device of claim 1, wherein sheath shaft includes a distal end portion, the distal end portion including a laser-resistant material.

3. The sheath device of claim 1, further comprising:
an elongated member configured to be inserted to the lumen of the sheath shaft.

4. The sheath device of claim 3, wherein the elongated member includes a dilator, a spacer, a biasing member, or a medical device, the medical device including a retrieval device or a pressure and temperature sensor.

5. The sheath device of claim 1, wherein the lumen of the elastic member is configured to receive a shaft of an insertion device.

6. The sheath device of claim 1, wherein the arm portion is a first arm portion, the connector including a second arm portion that extends from the sidewall of the connector.

7. The sheath device of claim 6, wherein the first arm portion is curved, and the second arm portion is straight.

8. The sheath device of claim 1, wherein the valve assembly includes a cap member, the cap member configured to move with respect to the connector to open or close the lumen of the elastic member.

9. The sheath device of claim 8, wherein the cap member includes a cylindrical portion, the cylindrical portion including a plunger defining a lumen, the plunger configured to contact the elastic member.

10. The sheath device of claim 8, wherein the connector includes a snap ramp, and the cap member includes a snap hook configured to be engaged with the snap ramp.

11. The sheath device of claim 1, wherein the arm portion includes a governing hole.

12. A sheath device comprising:
a sheath shaft defining a lumen, the sheath shaft configured to be inserted into a body of a patient;
a biasing member having a first portion disposed within the sheath shaft; and
a valve assembly coupled to a proximal end portion of the sheath shaft, the valve assembly including a connector and an elastic member, the elastic member being coupled to the connector, the connector including an arm portion that extends from a sidewall of the connector, a second portion of the biasing member being disposed within the arm portion, the elastic member configured to operate as a valve, the elastic member defining a lumen configured to receive a shaft of an insertion device.

13. The sheath device of claim 12, wherein the sheath shaft includes a distal end portion, the distal end portion including a laser-resistant material.

14. The sheath device of claim 12, wherein the sheath shaft includes a distal end portion, the distal end portion including a transparent portion.

15. The sheath device of claim 12, further comprising:
an indicator marking disposed on the connector, the indicator marking including an arrow.

16. The sheath device of claim 12, wherein the arm portion is a first arm portion, the connector including a second arm portion.

17. A method of inserting a sheath device into a body of a patient, the method comprising:
inserting a portion of a sheath device into a body of a patient, the sheath device including a sheath shaft, a connector having an arm portion, and an elastic member defining a lumen;
inserting a shaft of an insertion device into a lumen of the sheath shaft via the lumen of the elastic member, the insertion device including a laser fiber;
activating the laser fiber; and
removing one or more stone fragments via the sheath shaft and the arm portion.

18. The method of claim 17, further comprising:
moving a tip portion of the shaft of the insertion device to a location proximal to a tip portion of the sheath shaft; and
suctioning a stone into a distal end portion of the sheath shaft to fragment the stone using the laser fiber from a location within the distal end portion of the sheath shaft.

19. The method of claim 17, further comprising:
extending a tip portion of the laser fiber to a location distal to a tip portion of the sheath shaft; and
activating the laser fiber to fragment a stone.

20. The method of claim 17, wherein the arm portion is a first arm portion, the connector including a second arm portion, the method further comprising:
inserting a biasing member into the sheath shaft via the second arm portion, the biasing member configured to bias the sheath shaft away from the shaft of the insertion device within the sheath shaft.
